SEQUENCE LISTING (143 sequences)

SEQ ID NO: 1-2 - skipped (2 entries)

SEQ ID NO: 3 - DNA, 15 bp, synthetic construct
  source 1..15: mol_type = other DNA
aagaagtaaagggag

SEQ ID NO: 4-8 - skipped (5 entries)

SEQ ID NO: 9 - DNA, 14 bp, synthetic construct
  source 1..14: mol_type = other DNA
tttcttcattagcg

SEQ ID NO: 10 - DNA, 14 bp, synthetic construct
  source 1..14: mol_type = other DNA
ttataaagtgtcca

SEQ ID NO: 11 - DNA, 14 bp, synthetic construct
  source 1..14: mol_type = other DNA
tttcaatgtatggc

SEQ ID NO: 12 - DNA, 14 bp, synthetic construct
  source 1..14: mol_type = other DNA
ttataatggatggg

SEQ ID NO: 13 - DNA, 14 bp, synthetic construct
  source 1..14: mol_type = other DNA
ttaaaaagttcgag

SEQ ID NO: 14 - DNA, 14 bp, synthetic construct
  source 1..14: mol_type = other DNA
ttataacagaatcg

SEQ ID NO: 15 - DNA, 14 bp, synthetic construct
  source 1..14: mol_type = other DNA
ttttatgttctgct

SEQ ID NO: 16 - DNA, 14 bp, synthetic construct
  source 1..14: mol_type = other DNA
ttatagtgattgga

SEQ ID NO: 17 - DNA, 14 bp, synthetic construct
  source 1..14: mol_type = other DNA
ttatttagtgtagc

SEQ ID NO: 18 - DNA, 14 bp, synthetic construct
  source 1..14: mol_type = other DNA
ttatgttaatgggt

SEQ ID NO: 19 - DNA, 14 bp, synthetic construct
  source 1..14: mol_type = other DNA
ttataatcatgctc

SEQ ID NO: 20 - DNA, 14 bp, synthetic construct
  source 1..14: mol_type = other DNA
ttatatgatctccg

SEQ ID NO: 21 - DNA, 11 bp, synthetic construct
  source 1..11: mol_type = other DNA
ttatacatcta

SEQ ID NO: 22 - DNA, 11 bp, synthetic construct
  source 1..11: mol_type = other DNA
tttcttcatta

SEQ ID NO: 23 - DNA, 11 bp, synthetic construct
  source 1..11: mol_type = other DNA
tttcaatgtat

SEQ ID NO: 24 - skipped

SEQ ID NO: 25 - DNA, 19 bp, synthetic construct
  source 1..19: mol_type = other DNA
tttcctctctctctctctc

SEQ ID NO: 26 - DNA, 19 bp, synthetic construct
  source 1..19: mol_type = other DNA
ttcaacacacacacacaca

SEQ ID NO: 27 - DNA, 17 bp, synthetic construct
  source 1..17: mol_type = other DNA
cctcgctgaacccctta

SEQ ID NO: 28 - DNA, 10 bp, synthetic construct
  source 1..10: mol_type = other DNA
cgctaatgaa

SEQ ID NO: 29 - DNA, 17 bp, synthetic construct
  source 1..17: mol_type = other DNA
cctcgctgaacccctta

SEQ ID NO: 30 - DNA, 10 bp, synthetic construct
  source 1..10: mol_type = other DNA
tggacacttt

SEQ ID NO: 31 - DNA, 17 bp, synthetic construct
  source 1..17: mol_type = other DNA
cctcgctgaacccctta

SEQ ID NO: 32 - DNA, 10 bp, synthetic construct
  source 1..10: mol_type = other DNA
gccatacatt

SEQ ID NO: 33 - DNA, 17 bp, synthetic construct
  source 1..17: mol_type = other DNA
cctcgctgaacccctta

SEQ ID NO: 34 - DNA, 10 bp, synthetic construct
  source 1..10: mol_type = other DNA
cccatccatt

SEQ ID NO: 35 - DNA, 17 bp, synthetic construct
  source 1..17: mol_type = other DNA
cctcgctgaacccctta

SEQ ID NO: 36 - DNA, 10 bp, synthetic construct
  source 1..10: mol_type = other DNA
ctcgaacttt

SEQ ID NO: 37 - DNA, 17 bp, synthetic construct
  source 1..17: mol_type = other DNA
cctcgctgaacccctta

SEQ ID NO: 38 - DNA, 10 bp, synthetic construct
  source 1..10: mol_type = other DNA
cgattctgtt

SEQ ID NO: 39 - DNA, 17 bp, synthetic construct
  source 1..17: mol_type = other DNA
cctcgctgaacccctta

SEQ ID NO: 40 - DNA, 10 bp, synthetic construct
  source 1..10: mol_type = other DNA
agcagaacat

SEQ ID NO: 41 - DNA, 17 bp, synthetic construct
  source 1..17: mol_type = other DNA
cctcgctgaacccctta

SEQ ID NO: 42 - DNA, 10 bp, synthetic construct
  source 1..10: mol_type = other DNA
tccaatcact

SEQ ID NO: 43 - DNA, 17 bp, synthetic construct
  source 1..17: mol_type = other DNA
cctcgctgaacccctta

SEQ ID NO: 44 - DNA, 10 bp, synthetic construct
  source 1..10: mol_type = other DNA
gctacactaa

SEQ ID NO: 45 - DNA, 17 bp, synthetic construct
  source 1..17: mol_type = other DNA
cctcgctgaacccctta

SEQ ID NO: 46 - DNA, 10 bp, synthetic construct
  source 1..10: mol_type = other DNA
acccattaac

SEQ ID NO: 47 - DNA, 17 bp, synthetic construct
  source 1..17: mol_type = other DNA
cctcgctgaacccctta

SEQ ID NO: 48 - DNA, 10 bp, synthetic construct
  source 1..10: mol_type = other DNA
gagcatgatt

SEQ ID NO: 49 - DNA, 17 bp, synthetic construct
  source 1..17: mol_type = other DNA
cctcgctgaacccctta

SEQ ID NO: 50 - DNA, 10 bp, synthetic construct
  source 1..10: mol_type = other DNA
cggagatcat

SEQ ID NO: 51 - skipped

SEQ ID NO: 52 - DNA, 10 bp, synthetic construct
  source 1..10: mol_type = other DNA
cgctaatgaa

SEQ ID NO: 53 - skipped

SEQ ID NO: 54 - DNA, 10 bp, synthetic construct
  source 1..10: mol_type = other DNA
tggacacttt

SEQ ID NO: 55 - skipped

SEQ ID NO: 56 - DNA, 10 bp, synthetic construct
  source 1..10: mol_type = other DNA
gccatacatt

SEQ ID NO: 57 - skipped

SEQ ID NO: 58 - DNA, 10 bp, synthetic construct
  source 1..10: mol_type = other DNA
cccatccatt

SEQ ID NO: 59 - skipped

SEQ ID NO: 60 - DNA, 10 bp, synthetic construct
  source 1..10: mol_type = other DNA
ctcgaacttt

SEQ ID NO: 61 - skipped

SEQ ID NO: 62 - DNA, 10 bp, synthetic construct
  source 1..10: mol_type = other DNA
cgattctgtt

SEQ ID NO: 63 - skipped

SEQ ID NO: 64 - DNA, 10 bp, synthetic construct
  source 1..10: mol_type = other DNA
agcagaacat

SEQ ID NO: 65 - skipped

SEQ ID NO: 66 - DNA, 10 bp, synthetic construct
  source 1..10: mol_type = other DNA
tccaatcact

SEQ ID NO: 67 - skipped

SEQ ID NO: 68 - DNA, 10 bp, synthetic construct
  source 1..10: mol_type = other DNA
gctacactaa

SEQ ID NO: 69 - skipped

SEQ ID NO: 70 - DNA, 10 bp, synthetic construct
  source 1..10: mol_type = other DNA
acccattaac

SEQ ID NO: 71 - skipped

SEQ ID NO: 72 - DNA, 10 bp, synthetic construct
  source 1..10: mol_type = other DNA
gagcatgatt

SEQ ID NO: 73 - skipped

SEQ ID NO: 74 - DNA, 10 bp, synthetic construct
  source 1..10: mol_type = other DNA
cggagatcat

SEQ ID NO: 75 - DNA, 19 bp, synthetic construct
  source 1..19: mol_type = other DNA
accaccaccaccaccacca

SEQ ID NO: 76 - DNA, 10 bp, synthetic construct
  source 1..10: mol_type = other DNA
cgctaatgaa

SEQ ID NO: 77 - DNA, 19 bp, synthetic construct
  source 1..19: mol_type = other DNA
accaccaccaccaccacca

SEQ ID NO: 78 - DNA, 10 bp, synthetic construct
  source 1..10: mol_type = other DNA
tggacacttt

SEQ ID NO: 79 - DNA, 19 bp, synthetic construct
  source 1..19: mol_type = other DNA
accaccaccaccaccacca

SEQ ID NO: 80 - DNA, 10 bp, synthetic construct
  source 1..10: mol_type = other DNA
gccatacatt

SEQ ID NO: 81 - DNA, 19 bp, synthetic construct
  source 1..19: mol_type = other DNA
accaccaccaccaccacca

SEQ ID NO: 82 - DNA, 10 bp, synthetic construct
  source 1..10: mol_type = other DNA
cccatccatt

SEQ ID NO: 83 - DNA, 19 bp, synthetic construct
  source 1..19: mol_type = other DNA
accaccaccaccaccacca

SEQ ID NO: 84 - DNA, 10 bp, synthetic construct
  source 1..10: mol_type = other DNA
ctcgaacttt

SEQ ID NO: 85 - DNA, 19 bp, synthetic construct
  source 1..19: mol_type = other DNA
accaccaccaccaccacca

SEQ ID NO: 86 - DNA, 10 bp, synthetic construct
  source 1..10: mol_type = other DNA
cgattctgtt

SEQ ID NO: 87 - DNA, 19 bp, synthetic construct
  source 1..19: mol_type = other DNA
accaccaccaccaccacca

SEQ ID NO: 88 - DNA, 10 bp, synthetic construct
  source 1..10: mol_type = other DNA
agcagaacat

SEQ ID NO: 89 - DNA, 19 bp, synthetic construct
  source 1..19: mol_type = other DNA
accaccaccaccaccacca

SEQ ID NO: 90 - DNA, 10 bp, synthetic construct
  source 1..10: mol_type = other DNA
tccaatcact

SEQ ID NO: 91 - DNA, 19 bp, synthetic construct
  source 1..19: mol_type = other DNA
accaccaccaccaccacca

SEQ ID NO: 92 - DNA, 10 bp, synthetic construct
  source 1..10: mol_type = other DNA
gctacactaa

SEQ ID NO: 93 - DNA, 19 bp, synthetic construct
  source 1..19: mol_type = other DNA
accaccaccaccaccacca

SEQ ID NO: 94 - DNA, 10 bp, synthetic construct
  source 1..10: mol_type = other DNA
acccattaac

SEQ ID NO: 95 - DNA, 19 bp, synthetic construct
  source 1..19: mol_type = other DNA
accaccaccaccaccacca

SEQ ID NO: 96 - DNA, 10 bp, synthetic construct
  source 1..10: mol_type = other DNA
gagcatgatt

SEQ ID NO: 97 - DNA, 19 bp, synthetic construct
  source 1..19: mol_type = other DNA
accaccaccaccaccacca

SEQ ID NO: 98 - DNA, 10 bp, synthetic construct
  source 1..10: mol_type = other DNA
cggagatcat

SEQ ID NO: 99 - DNA, 10 bp, synthetic construct
  source 1..10: mol_type = other DNA
ttcattagcg

SEQ ID NO: 100 - DNA, 10 bp, synthetic construct
  source 1..10: mol_type = other DNA
aaagtgtcca

SEQ ID NO: 101 - DNA, 10 bp, synthetic construct
  source 1..10: mol_type = other DNA
aatgtatggc

SEQ ID NO: 102 - DNA, 10 bp, synthetic construct
  source 1..10: mol_type = other DNA
aatggatggg

SEQ ID NO: 103 - DNA, 10 bp, synthetic construct
  source 1..10: mol_type = other DNA
aaagttcgag

SEQ ID NO: 104 - DNA, 10 bp, synthetic construct
  source 1..10: mol_type = other DNA
aacagaatcg

SEQ ID NO: 105 - DNA, 10 bp, synthetic construct
  source 1..10: mol_type = other DNA
atgttctgct

SEQ ID NO: 106 - DNA, 10 bp, synthetic construct
  source 1..10: mol_type = other DNA
agtgattgga

SEQ ID NO: 107 - DNA, 10 bp, synthetic construct
  source 1..10: mol_type = other DNA
ttagtgtagc

SEQ ID NO: 108 - DNA, 10 bp, synthetic construct
  source 1..10: mol_type = other DNA
gttaatgggt

SEQ ID NO: 109 - DNA, 10 bp, synthetic construct
  source 1..10: mol_type = other DNA
aatcatgctc

SEQ ID NO: 110 - DNA, 10 bp, synthetic construct
  source 1..10: mol_type = other DNA
atgatctccg

SEQ ID NO: 111 - DNA, 10 bp, synthetic construct
  source 1..10: mol_type = other DNA
ttcattagcg

SEQ ID NO: 112 - DNA, 10 bp, synthetic construct
  source 1..10: mol_type = other DNA
aaagtgtcca

SEQ ID NO: 113 - DNA, 10 bp, synthetic construct
  source 1..10: mol_type = other DNA
aatgtatggc

SEQ ID NO: 114 - DNA, 10 bp, synthetic construct
  source 1..10: mol_type = other DNA
aatggatggg

SEQ ID NO: 115 - DNA, 10 bp, synthetic construct
  source 1..10: mol_type = other DNA
aaagttcgag

SEQ ID NO: 116 - DNA, 10 bp, synthetic construct
  source 1..10: mol_type = other DNA
aacagaatcg

SEQ ID NO: 117 - DNA, 10 bp, synthetic construct
  source 1..10: mol_type = other DNA
atgttctgct

SEQ ID NO: 118 - DNA, 10 bp, synthetic construct
  source 1..10: mol_type = other DNA
agtgattgga

SEQ ID NO: 119 - DNA, 10 bp, synthetic construct
  source 1..10: mol_type = other DNA
ttagtgtagc

SEQ ID NO: 120 - DNA, 10 bp, synthetic construct
  source 1..10: mol_type = other DNA
gttaatgggt

SEQ ID NO: 121 - DNA, 10 bp, synthetic construct
  source 1..10: mol_type = other DNA
aatcatgctc

SEQ ID NO: 122 - DNA, 10 bp, synthetic construct
  source 1..10: mol_type = other DNA
atgatctccg

SEQ ID NO: 123 - DNA, 10 bp, synthetic construct
  source 1..10: mol_type = other DNA
ttcattagcg

SEQ ID NO: 124 - DNA, 10 bp, synthetic construct
  source 1..10: mol_type = other DNA
aaagtgtcca

SEQ ID NO: 125 - DNA, 10 bp, synthetic construct
  source 1..10: mol_type = other DNA
aatgtatggc

SEQ ID NO: 126 - DNA, 10 bp, synthetic construct
  source 1..10: mol_type = other DNA
aatggatggg

SEQ ID NO: 127 - DNA, 10 bp, synthetic construct
  source 1..10: mol_type = other DNA
aaagttcgag

SEQ ID NO: 128 - DNA, 10 bp, synthetic construct
  source 1..10: mol_type = other DNA
aacagaatcg

SEQ ID NO: 129 - DNA, 10 bp, synthetic construct
  source 1..10: mol_type = other DNA
atgttctgct

SEQ ID NO: 130 - DNA, 10 bp, synthetic construct
  source 1..10: mol_type = other DNA
agtgattgga

SEQ ID NO: 131 - DNA, 10 bp, synthetic construct
  source 1..10: mol_type = other DNA
ttagtgtagc

SEQ ID NO: 132 - DNA, 10 bp, synthetic construct
  source 1..10: mol_type = other DNA
gttaatgggt

SEQ ID NO: 133 - DNA, 10 bp, synthetic construct
  source 1..10: mol_type = other DNA
aatcatgctc

SEQ ID NO: 134 - DNA, 10 bp, synthetic construct
  source 1..10: mol_type = other DNA
atgatctccg

SEQ ID NO: 135 - DNA, 6867 bp, synthetic construct
  source 1..6867: mol_type = other DNA
ttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggct

SEQ ID NO: 136 - DNA, 40 bp, synthetic construct
  source 1..40: mol_type = other DNA
attaagtttaccgagctcgaattcgggaaacctgtcgtgc

SEQ ID NO: 137 - DNA, 40 bp, synthetic construct
  source 1..40: mol_type = other DNA
ataagggaaccggatattcattacgtcaggacgttgggaa

SEQ ID NO: 138 - DNA, 38 bp, synthetic construct
  source 1..38: mol_type = other DNA
gcgatcggcaattccacacaacaggtgcctaatgagtg

SEQ ID NO: 139 - DNA, 38 bp, synthetic construct
  source 1..38: mol_type = other DNA
ttgtgtcgtgacgagaaacaccaaatttcaactttaat

SEQ ID NO: 140 - DNA, 40 bp, synthetic construct
  source 1..40: mol_type = other DNA
attcatttttgtttggattatactaagaaaccaccagaag

SEQ ID NO: 141 - DNA, 40 bp, synthetic construct
  source 1..40: mol_type = other DNA
caccctcagaaaccatcgatagcattgagccatttgggaa

SEQ ID NO: 142 - DNA, 40 bp, synthetic construct
  source 1..40: mol_type = other DNA
aacaataacgtaaaacagaaataaaaatcctttgcccgaa

SEQ ID NO: 143 - DNA, 40 bp, synthetic construct
  source 1..40: mol_type = other DNA
agccaccactgtagcgcgttttcaagggagggaaggtaaa

(19) United States
(12) Patent Application Publication
(10) Pub. No.: US 2026/0185941 A1
(43) Pub. Date: Jul. 2, 2026

(54) METHODS AND KITS FOR MICROSCOPIC IMAGING

(71) Applicant: YALE UNIVERSITY, New Haven, CT (US)

(72) Inventors: Florian Schueder, New Haven, CT (US); Joerg Bewersdorf, New Haven, CT (US)

(21) Appl. No.: 19/131,332

(22) PCT Filed: Nov. 21, 2023
(86) PCT No.: PCT/US2023/080766

§ 371 Date: May 20, 2025

Related U.S. Application Data
(60) Provisional application No. 63/427,212, filed on Nov. 22, 2022.

(51) Int. Cl. G01N 21/64; G01N 21/65; G01N 33/53

(52) U.S. Cl. CPC G01N 21/6458; G01N 21/6428; G01N 21/65; G01N 33/5308; G01N 2021/6441

(57) ABSTRACT

Described herein is an imaging method, the method includes: performing a first labeling of a sample and acquiring a first image. Performing the first labeling includes applying to a sample a first target; applying to the sample a first adapter; and applying to the sample a first imaging molecule including a first detection motif. Acquiring the first image includes acquiring a first image the first detection motif. The first target labels a point of interest, such as a molecule, a complex, a structure, an organelle or a cell, in the sample. The first adapter mediates a specific, indirect and reversible interaction between the first imaging molecule and the first target.

### CROSS-REFERENCE TO RELATED APPLICATIONS

The present application claims priority under 35 U.S.C. § 119 (e) to U.S. Provisional Patent Application No. 63/427,212, filed Nov. 22, 2022, which is incorporated herein by reference in its entirety.

### STATEMENT REGARDING FEDERALLY SPONSORED RESEARCH OR DEVELOPMENT

This invention was made with government support under Contract No. P30 DK045735 awarded by the National Institutes of Health. The government has certain rights in the invention.

### SEQUENCE LISTING

The ASCII text file named “047162-7393WO1_Sequence Listing” created on Nov. 7, 2023, comprising 116 KB Kbytes, is hereby incorporated by reference in its entirety.

### BACKGROUND OF THE INVENTION

DNA probes as programable probes have revolutionized parts of fluorescence microscopy. For example, DNA-PAINT has emerged as one of the most promising super-resolution microscopy methods in the last couple of years. Conventional DNA-PAINT technology, however; has relatively slow imaging speed, is susceptible to background and can have limited multiplexing potential.

Therefore, there is a need for imaging technologies that enjoy the advantages of DNA-PAINT, but do not suffer from its problems. The present invention addresses this need.

### SUMMARY

In some aspects, the present invention is directed to the following non-limiting embodiments:

### Method of Microscopy Imaging

In some aspects, the present invention is directed to a method of microscopy imaging.

In some embodiments, the method comprises: exposing a sample having a plurality of targets to a plurality of transient single-strand-nucleic-acid adapter molecules; exposing the sample to a plurality of single-strand-nucleic-acid imaging molecules; and exposing the sample to an illumination source having a wavelength capable of interacting with the plurality of single-strand-nucleic-acid imaging molecules.

In some embodiments, the transient single-strand-nucleic-acid adapter molecules comprise: a first region having a target-complementary sequence; and a second region having a single-strand-nucleic-acid-imaging-molecule-complementary sequence.

In some embodiments, the plurality of transient single-strand-nucleic-acid adapter molecules have a quantity greater than an estimated or actual quantity of targets.

In some embodiments, the plurality of transient single-strand-nucleic-acid adapter molecules have a quantity or concentration greater than the plurality of single-strand-nucleic-acid imaging molecules.

In some embodiments, the plurality of transient single-strand-nucleic-acid adapter molecules have a quantity or concentration greater than the plurality of single-strand-nucleic-acid imaging molecules by a ratio selected from the group consisting of: at least about 1; at least about 10; and at least about 100.

In some embodiments, the plurality of transient single-strand-nucleic-acid adapter molecules have a quantity or concentration greater than the plurality of single-strand-nucleic-acid imaging molecules by a ratio of about 500.

In some embodiments, the target-complementary sequence is less than 11 nucleotides.

In some embodiments, the target-complementary sequence is selected from the group consisting of: between 6 and 10 nucleotides and between 8 and 10 nucleotides.

In some embodiments, the method further comprises: exposing the sample to an eraser molecule adapted and configured to quench the transient single-strand-nucleic-acid adapter molecules; exposing the sample to a second plurality of transient single-strand-nucleic-acid adapter molecules having a second, different target-complementary sequence; exposing the sample to the plurality of single-strand-nucleic-acid imaging molecules; and exposing the sample to an illumination source having a wavelength capable of interacting with the plurality of single-strand-nucleic-acid imaging molecules.

In some embodiments, the method is performed without rinsing the plurality of transient single-strand-nucleic-acid adapter molecules from the sample.

In some embodiments, the eraser molecule and the second plurality of transient single-strand-nucleic-acid adapter molecules are introduced simultaneously.

In some embodiments, the eraser molecule and the second plurality of transient single-strand-nucleic-acid adapter molecules are introduced sequentially.

In some embodiments, the plurality of single-strand-nucleic-acid imaging molecules include a speed-optimized sequence.

In some embodiments, the plurality of single-strand-nucleic-acid imaging molecules are fluorogenic.

In some embodiments, the plurality of single-strand-nucleic-acid imaging molecules are fluorescent; and the detected change in light is fluorescence emitted by the single-strand-nucleic-acid imaging molecules.

In some embodiments, the single-strand-nucleic-acid imaging molecules are detected individually in order to generate a single-molecule localization super-resolution microscopy image.

In some embodiments, the sample is a biological tissue section.

In some embodiments, the plurality of targets are antibodies or binding ligands that bind to a plurality of specific proteins in the sample; and each type of antibody or binding ligand is conjugated to a different single-strand nucleic acid.

In some embodiments, the single-strand nucleic acids are RNA or DNA molecules.

In some embodiments, the single-strand-nucleic-acid imaging molecules comprise a single-strand nucleic acid coupled to a molecule exhibiting a Raman signature detectable by a Raman microscopy.

In some embodiments, the single-strand-nucleic-acid imaging molecules comprise a single-strand nucleic acid coupled to a nanoparticle.

In some embodiments, the nanoparticle is a gold nanoparticle.

In some embodiments, the interaction is scattering.

### Kit

In some aspects, the present invention is directed to a kit.

In some embodiments, the kit comprises: a plurality of transient single-strand-nucleic-acid adapter molecules; and a plurality of single-strand-nucleic-acid imaging molecules.

In some embodiments, the transient single-strand-nucleic-acid adapter molecules comprise: a first region having a target-complementary sequence; and a second region having a single-strand-nucleic-acid-imaging-molecule-complementary sequence.

In some embodiments, the plurality of transient single-strand-nucleic-acid adapter molecules have a quantity or concentration greater than the plurality of single-strand-nucleic-acid imaging molecules.

In some embodiments, the plurality of transient single-strand-nucleic-acid adapter molecules have a quantity or concentration greater than the plurality of single-strand-nucleic-acid imaging molecules by a ratio selected from the group consisting of: at least about 1; at least about 10; and at least about 100.

In some embodiments, the plurality of transient non-fluorescent single-strand-nucleic-acid adapter molecules have a quantity or concentration greater than the plurality of fluorescent imaging molecules by a ratio of about 500.

### Imaging Method

In some aspects, the present invention is directed to an imaging method.

In some embodiments, the method comprising performing a first labeling; and acquiring a first image.

In some embodiments, performing the first labeling comprises: applying to a sample one or more targets comprising a first target, which comprises a first target single-strand-nucleic-acid; applying to the sample a first adapter comprising a first adapter single-strand-nucleic-acid; and applying to the sample a first imaging molecule comprising a first imaging molecule single-strand-nucleic-acid and a first detection motif.

In some embodiments, acquiring the first image comprises acquiring a first image of the first detection motif.

In some embodiments, the first adapter single-strand-nucleic-acid comprises: a first region having a sufficient sequence complementarity to bind the target single-strand-nucleic-acid; and a second region having a sufficient sequence complementarity to bind the first imaging molecule single-strand-nucleic-acid.

In some embodiments, the first adapter binds the target and the first imaging molecule.

In some embodiments, the first target comprises the first target single-strand-nucleic-acid attached to an antibody or a polypeptide that specifically binds to a point of interest, optionally a protein, a protein complex, a nucleic acid, a cell structure, a cell organelle, or a cell, in the sample.

In some embodiments, the first target comprises the first target single-strand-nucleic-acid attached to a targeting nucleic acid that specifically binds to or is complementary with a point of interest, optionally a nucleic acid, in the sample.

In some embodiments, the first detection motif is a fluorescence motif, optionally a fluorescent protein, a fluorescent small molecule, or a quantum dot.

In some embodiments, the first detection motif is a metal nanoparticle, optionally a gold nanoparticle.

In some embodiments, the first detection motif is a Raman scattering motif, optionally a Raman dye, optionally a Raman dye suitable for a stimulated Raman scattering microscopy.

In some embodiments, the first detection motif is an isotope.

In some embodiments, a number of complementary base pairs between the first target single-strand-nucleic-acid and the first region of the first adapter ranges between 1-30.

In some embodiments, a number of complementary base pairs between the first target single-strand-nucleic-acid and the first region of the first adapter ranges between 5-20.

In some embodiments, a number of complementary base pairs between the first target single-strand-nucleic-acid and the first region of the first adapter ranges between 8-12.

In some embodiments, a Kₒₙ between the first target single-strand-nucleic-acid and the first region of the adapter ranges between 1*10⁴ l/M*s and 1*10⁷ l/M*s.

In some embodiments, a K_{off} between the first target single-strand-nucleic-acid and the first region of the adapter ranges between 1 l/s and 0.0001 l/s.

In some embodiments, a K_{d} between the first target single-strand-nucleic-acid and the first region of the adapter ranges between 10 UM and 1 nM.

In some embodiments, a number of complementary base pairs between the first imaging molecule single-strand-nucleic-acid and the second region of the adapter ranges between 1 and 30.

In some embodiments, a number of complementary base pairs between the first imaging molecule single-strand-nucleic-acid and the second region of the adapter ranges between 5 and 20.

In some embodiments, a number of complementary base pairs between the first imaging molecule single-strand-nucleic-acid and the second region of the adapter ranges between 8 and 12.

In some embodiments, a Kₒₙ between the first imaging molecule single-strand-nucleic-acid and the second region of the adapter ranges between 1*10⁴ l/M*s and 1*10⁷ l/M*s.

In some embodiments, a K_{off} between the first imaging molecule single-strand-nucleic-acid and the second region of the adapter ranges between 1000 l/s and 0.0001 l/s.

In some embodiments, a K_{d} between the first imaging molecule single-strand-nucleic-acid and the second region of the adapter ranges between 10 UM and 1 nM.

In some embodiments, performing the first labeling comprises: applying to the sample a plurality of first targets, each comprising a first target single-strand-nucleic-acid; applying to the sample a plurality of first adapters, each comprising a first adapter single-strand-nucleic-acid; and applying to the sample a plurality of first imaging molecules, each comprising a first imaging molecule single-strand-nucleic-acid and a first detection motif.

In some embodiments, acquiring the first image of the plurality of first detection motifs of the plurality of first imaging molecules.

In some embodiments, each of the first adapters mediates an association of each of the plurality of first targets and each of the plurality of the first imaging molecules in a sequence-specific manner.

In some embodiments, the plurality of first detection motifs do not interfere with each other during the acquisition of the first image.

In some embodiments, the method further comprises: applying to the sample an eraser molecule to disrupt the association between the first target and the first imaging molecule mediated by the first adapter; performing a second labeling; and acquiring a second image of the second detection motif. In some embodiments, performing a second labeling comprises: applying to a sample a second target comprising a second target single-strand-nucleic-acid; applying to the sample a second adapter comprising a second adapter single-strand-nucleic-acid; and applying to the sample a second imaging molecule comprising a second imaging molecule single-strand-nucleic-acid and a second detection motif.

In some embodiments, the one or more targets applied in the first labeling further comprises a second target comprising a second single-strand-nucleic-acid. In some embodiments, the method further comprises: applying to the sample an eraser molecule to disrupt the association between the first target and the first imaging molecule mediated by the first adapter; performing a second labeling; and acquiring a second image of the second detection motif. In some embodiments, performing the second labeling comprises: applying to the sample a second adapter comprising a second adapter single-strand-nucleic-acid; and applying to the sample a second imaging molecule comprising a second imaging molecule single-strand-nucleic-acid and a second detection motif.

In some embodiments, the second adapter single-strand-nucleic-acid comprises: a third region having a sufficient complementarity to bind the second target single-strand-nucleic-acid; and a fourth region having a sufficient complementarity to bind the second imaging molecule single-strand-nucleic-acid.

In some embodiments, the second adapter mediates an association between the second target and the second imaging molecule.

In some embodiments, the eraser molecule comprises an eraser molecule single-strand-nucleic-acid having a sufficient sequence complementarity to bind the first region or the second region of the first adapter, and the eraser molecule prevents the hybridization between the first target single-strand-nucleic-acid and the first region of the adapter, and/or prevents the hybridization between the first imaging molecule single-strand-nucleic-acid and the second region of the adapter.

In some embodiments, the first target, the first adapter, the first imaging molecule and the eraser molecule are not washed away from the sample before the application of the second target, the second adapter, and the second imaging molecule.

In some embodiments, the one or more targets, the first adapter, the first imaging molecule and the eraser molecule are not washed away from the sample before the application of the second adapter, and the second imaging molecule.

In some embodiments, a signal of the first detection motif and a signal of the second detection motif overlap or are the same.

In some embodiments, in each of the first labeling and the second labeling, 4 or more of different detection motifs having different signals are used.

In some embodiments, the sample is expanded according to an expansion microscopy technology.

### Device

In some aspects, the present invention is directed to a device.

In some embodiments, the device comprises: a sample holder for holding a sample; a computer-operated liquid applicator for applying a liquid to the sample; a computer-operated microscope; and a computer.

In some embodiments, the computer is programmed to operate the liquid applicator to perform a first application of: one ore more targets, which comprises a first target for specifically binding to a first component in the sample; a first imaging molecule comprising a first detection motif detectable by the microscope; and a first adapter for mediating an association between the first target and the first imaging molecule.

In some embodiments, the computer is further programmed to operate the microscope to record a first signal of the first detection motif,

In some embodiments, the computer is further programmed to operate the liquid applicator to perform a second application.

In some embodiments, the second application comprises the application of: an eraser molecule for interrupting the first adapter-mediated interaction between the first target and the first adapter; a second target for specifically binding to a second component in the sample; a second imaging molecule comprising a second detection motif detectable by the microscope; and a second adapter for mediating an association between the second target and the second imaging molecule.

In some embodiments, the one or more targets further comprises a second target for specifically binding to a second component in the sample, and the second application comprises the application of: an eraser molecule for interrupting the first adapter-mediated interaction between the first target and the first adapter; a second imaging molecule comprising a second detection motif detectable by the microscope; and a second adapter for mediating an association between the second target and the second imaging molecule.

In some embodiments, the computer is further programmed to operate the microscope to record a second signal of the second detection motif.

In some embodiments, the first application, the recording of the first signal, the second application, and the recording of the second signal are performed sequentially in this order.

In some embodiments, the device does not remove the liquid applied in the first application before performing the second application and/or recording of the second signal.

In some embodiments, the first signal and the second signal overlap with each other or are identical.

In some embodiments, the first detection motif and the second detection motif are the first detection motif or the second detection motif is a fluorescence motif, optionally a fluorescent protein, a fluorescent small molecule, or a quantum dot.

In some embodiments, the first detection motif and the second detection motif are the first detection motif or the second detection motif is a metal nanoparticle, optionally a gold nanoparticle.

In some embodiments, the first detection motif and the second detection motif are the first detection motif or the second detection motif is a Raman scattering motif, optionally a Raman dye, optionally a Raman dye suitable for a stimulated Raman scattering microscopy.

In some embodiments, the first detection motif and the second detection motif are the first detection motif or the second detection motif is an isotope.

In some embodiments, the device further comprises a reservoir for storing the one or more targets, the first adapter, the first imaging molecule, the eraser, the second target, the second adapter, the second imaging molecule.

In some embodiments, the method further comprises at least one selected from the group consisting of the first target, the first adapter, the first imaging molecule, the eraser, the second target, the second adapter, and the second imaging molecule.

In some embodiments, the first target comprises a first target single-strand-nucleic-acid.

In some embodiments, the first adapter comprises a first adapter single-strand-nucleic-acid.

In some embodiments, the first imaging molecule comprises a first imaging molecule single-strand-nucleic-acid attached to the first detection motif.

In some embodiments, the eraser molecule comprises an eraser molecule single-strand-nucleic-acid having a sufficient sequence complementarity to bind the first region or the second region of the first adapter.

In some embodiments, the second target comprises a second target single-strand-nucleic-acid.

In some embodiments, the second adapter comprises a second adapter single-strand-nucleic-acid.

In some embodiments, the second imaging molecule comprises a second imaging molecule single-strand-nucleic-acid attached to the second detection motif.

In some embodiments, the first adapter single-strand-nucleic-acid comprises: a first region having a sufficient sequence complementarity to bind the first target single strand nucleic acid; and a second region having a sufficient sequence complementarity to bind the first imaging molecule single-strand-nucleic-acid.

In some embodiments, the second adapter single-strand-nucleic-acid comprises: a third region having a sufficient sequence complementarity to bind the second target single strand nucleic acid; and a fourth region having a sufficient sequence complementarity to bind the second imaging molecule single-strand-nucleic-acid.

In some embodiments, the device comprises the first target, the first adapter, the first imaging molecule, the eraser, the second target, the second adapter, and the second imaging molecule.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1B provide a schematic of an imager binding to a target of interest (T1) via a transient adapter according to an embodiment of the invention.

FIGS. 2A-2B provide a schematic of an eraser strand according to an embodiment of the invention.

FIG. 3 (top panel) depicts how the same imager can be used with multiple adapters to image multiple targets of interest (over time, e.g., sequentially) according to an embodiment of the invention. FIG. 3 (bottom panel) depicts how adapters can share a common target-complementary region and support multiple imagers according to an embodiment of the invention.

FIGS. 4A-4D, 5A-5B and 6A-6G depict concentrations for various adapter binding lengths.

FIGS. 7A-7D and 8A-8D are super-resolution microscopy images using embodiments of the invention.

FIG. 9 depicts a method of single-molecule imaging according to an embodiment of the invention.

FIG. 10 depicts a system 1000 and a kit 1000a for single-molecule imaging according to an embodiment of the invention.

FIG. 11A is a time-lapse series of diffraction-limited imaging of cells according to an embodiment of the invention.

FIG. 11B is a time-lapse series of diffraction-limited imaging of mouse-spleen tissue showing the erasure of the signal from the CD45 protein according to an embodiment of the invention.

FIGS. 12A-12D are six-target DNA-PAINT image of the Golgi apparatus demonstrating spectrally unlimited super-resolved multiplexed imaging according to an embodiment of the invention.

FIGS. 13A-13E: Proof of concept of FLASH-PAINT, in accordance with some embodiments. FIG. 13A: Schematic representation of ‘classical’ DNA-PAINT using direct binding of Imagers to docking sites and FLASH-PAINT using Transient Adapters. FIG. 13B: Proof of concept with DNA origami nanostructures. Frame pattern DNA origamis are sampled via direct DNA-PAINT, 20-nm grid DNA origamis via Transient Adapters (FLASH-PAINT).

FIG. 13C: DNA origami experiment to compare association rates for direct binding and binding via Transient Adapters. FIG. 13D: Measured (data points) and calculated (curves) association rates for direct and adapter-mediated binding for different Transient Adapter concentrations. FIG. 13E: 4-plex imaging of 5-nm grid DNA origamis featuring binding sites arranged as letters (‘Y’, ‘A’, ‘L’, ‘E’). The data was acquired in 25 min per round and ˜100 min total imaging time. 121 to 246 super-resolution images of individual letters were averaged to generate the displayed letters. A representative field of view and individual DNA origami letters are shown in FIGS. 28-32. Scale bars: FIGS. 13A-13C: 100 nm; FIG. 13E: 20 nm.

FIGS. 14A-14C: Molecular target switching via a Transient Adapter-Eraser combination, in accordance with some embodiments. FIG. 14A: Schematic depiction of molecular target switching. Eraser E1 neutralizes in Round 2 Transient Adapter 1 while newly added Transient Adapter 2 directs the Imager probe to a new target. FIG. 14B: Quantification of switching efficiency using DNA origami (at the example of adapter sequence A19). In the first round of imaging only the Imager, not the Transient Adapter, is introduced. In the second round, both Transient Adapter (20 nM) and Imager are applied. Finally, the solution is replaced with an Eraser (100 nM) and an Imager. After a 3-min incubation period, the third round of imaging is carried out. FIG. 14C: Time course of switching the labeled molecular target from Tom20 on mitochondria to α-tubulin (microtubules) in a U-2 OS cell. The sample is in a medium containing Transient Adapter 1 to visualize mitochondria and an Imager pre acquisition. At the start of the acquisition Eraser 1 (to erase signal from mitochondria) and Transient Adapter 2 are added to the medium. Scale bars: 5 μm.

FIGS. 15A-15B: 9-target FLASH-PAINT image of a U-2 OS cell, in accordance with some embodiments. Nine different protein targets located at the Golgi complex, mitochondria, and the nucleus were imaged at super-resolution. The yellow, red, and green subpanels zoom in on mitochondria (yellow box), parts of the Golgi complex and nuclear envelope (red box), and a nucleolus (green box), respectively. For clarity, only a subset of proteins is shown in these subpanels; labels marked in gray are not shown. Scale bars: 5 μm (overview), 500 nm (zoom-ins).

FIGS. 16A-16G: 9-target FLASH-PAINT imaging of normal and bulbous-tip cilia in RPE-pHSmo cells, in accordance with some embodiments. FIGS. 16A-16B: FLASH-PAINT image of nine different protein targets at a normal (FIG. 16A; Cilium 1) and bulbous tip (FIG. 16B; Cilium 2) cilium. Quartiles along the length of the cilia are represented by square boxes. Subsets of the nine targets of the zoomed-in proximal and distal regions are shown in the blue and yellow boxes, respectively. The zoomed-in boxes labeled “Δz=50 nm” show 50-nm thick cross sections of the acquired 3D super-resolution data sets to highlight the distribution of acetylated tubulin (Actub) inside the cilia. The yellow and blue arrowheads point at the basal body distal appendage CEP164 and the transition zone (TZ) Rpgrip11 markers, respectively. Between the bulbous tip (asterisk in FIG. 16B) and a varicosity (white arrow in middle2 box in FIG. 16B), thinning of the ciliary membrane (white arrows in yellow boxes in FIG. 16B) can be observed. FIG. 16C Bar plots summarizing the axial distribution of the nine different targets in the two cilia. For each target, the median (line) and 25% and 75% quartiles (bottom and top of the bar) are indicated. FIG. 16D: Numbers of target clusters in each of the four regions for both cilia. FIG. 16E: Median distances of target clusters to the central acetylated-tubulin (Actub) filament in each of the four regions for both cilia. In the proximal regions, Actub and Glutub axoneme targets show the shortest distance to the filament (black dashed-line rectangles). FIGS. 16F-16G: Median distances between clusters of two different targets in the proximal (FIG. 16F) and distal (FIG. 16G) regions of both cilia Sept2 clusters are closer to all other targets in the normal cilium (Cilium 1) compared to the bulbous tip cilium (Cilium 2) (black dashed-line rectangles in FIG. 16F & 16G). In the distal region, a similar observation can be made for Ift88 (magenta dashed-line rectangles in FIG. 16G). Scale bars: 1 μm (overviews); 300 nm (zoom-ins).

FIGS. 17A-17P: 12-target FLASH-PAINT imaging of Golgi complexes in untreated and nocodazole-treated HeLa cells, in accordance with some embodiments. FIGS. 17A-17C: Overview of the Golgi complex and in the secretory pathway in a HeLa cell in interphase. Different subsets of protein targets are shown in the three images as indicated by the colored labels (gray-labeled targets not shown). FIG. 17D: 3D surface reconstruction of cis, medial, and trans cisternae of the Golgi complex. FIGS. 17E-17G: Zoomed in regions of the white boxes shown in FIGS. 17A-17C, respectively, highlighting a side view of a Golgi stack revealing the sequential organization of the stack into cis cisternae, medial cisternae, trans cisternae and TGN FIG. 17E, the spatial relationship between ERES, ERGIC and COPI and II vesicles FIG. 17F, and an en face view of a Golgi stack showing Giantin located at the Golgi rim FIG. 17G. FIG. 17H: Median distances between localization events of different targets. Only localization events closer than 500 nm to each other were considered. Median distances >100 nm are shown in blue.

FIGS. 171-17P: Representation equivalent to FIGS. 17A-17H of Golgi ministacks in a nocodazole-treated HeLa cell in interphase. Scale bars: 5 μm (FIGS. 17A-17C & 171-17K), 500 nm (FIGS. 17D-17G), 1 μm (FIGS. 17L-17O).

FIGS. 18A-18I: Volumetric multiplexed cellular organelle imaging at super-resolution using FLASH-PAINT, in accordance with some embodiments. FIG. 18A-18D: Depth projection of the ER (FIG. 18A; Sec61B), mitochondria (FIG. 18B; Tom20), lysosomes (FIG. 18C; Lamp1) and Golgi complex (FIG. 18D; ManII-GFP) in a ˜2.5-μm thick 4-plexed FLASH-PAINT data set of a HeLa cell. FIGS. 18E-18F: 3D rendering of localization data (FIG. 18E) and surface rendering (FIG. 18F) of the four labels. FIGS. 18G-18H: Number of contact sites (g; defined as distance <100 nm) and median area of contact sites (FIG. 18H) between Golgi complex, lysosomes, ER and mitochondria. FIG. 18I: Bar plots of the areas of the individual contact sites. The data points represent the areas of contact for all identified individual contacts between the organelles in the cell. For each type of contact, the median (line) and 25% and 75% quartiles (bottom and top of the bar) are indicated. Scale bars: 5 μm.

FIGS. 19A-19I: Experimental Workflow for Kinetics Measurements using DNA origami, in accordance with some embodiments. Two distinct DNA origami species are used simultaneously. The first DNA origami species features a single docking site for Imager probe binding via a Transient Adapter strand and orthogonal docking sites arranged in a frame pattern (FIG. 20). The second DNA origami species features a single docking site for direct Imager probe binding and the same frame pattern but with a docking sequence orthogonal to that used in the first DNA origami species. FIG. 19A: In the first imaging round, the single docking sites on both DNA origami species are imaged; the same Imager probe can bind to the first DNA origami species via a Transient Adapter and directly to the second DNA origami species. FIG. 19B: In the second imaging round, the frame of the first DNA origami species is imaged. FIG. 19C: In the third imaging round, the frame of the second DNA origami species is imaged. FIG. 19D: In the final imaging round, both frames of both DNA origami species are imaged. FIG. 19E: After applying standard single-molecule localization-based super-resolution microscopy post-processing techniques (i.e., localization fitting and drift correction), the first three imaging rounds are aligned with the last round. FIGS. 19F-19G: Using the frame images of the two DNA origami species, single docking sites are identified and binding kinetics are extracted for kinetics analysis for Transient Adapter-mediated (FIG. 19F) and direct (FIG. 19G) binding. FIG. 19H: Cut-out of exemplary field of view. Colors are assigned based on in which imaging round the signal was recorded. FIG. 19I: Schematic representation of direct and Transient Adapter-mediated Imager binding. Scale bar 100 nm.

FIG. 20: Used DNA origami designs, in accordance with some embodiments. Schematic representation of all DNA origami designs used in this study. The hexagons represent 3′-staple positions. Blue and red hexagons are representing two different staples extended with docking sites for transient binding of either Imager probes or Transient Adapters. The orange hexagons depict staples extended with a biotin modification for immobilization on the cover slip surface.

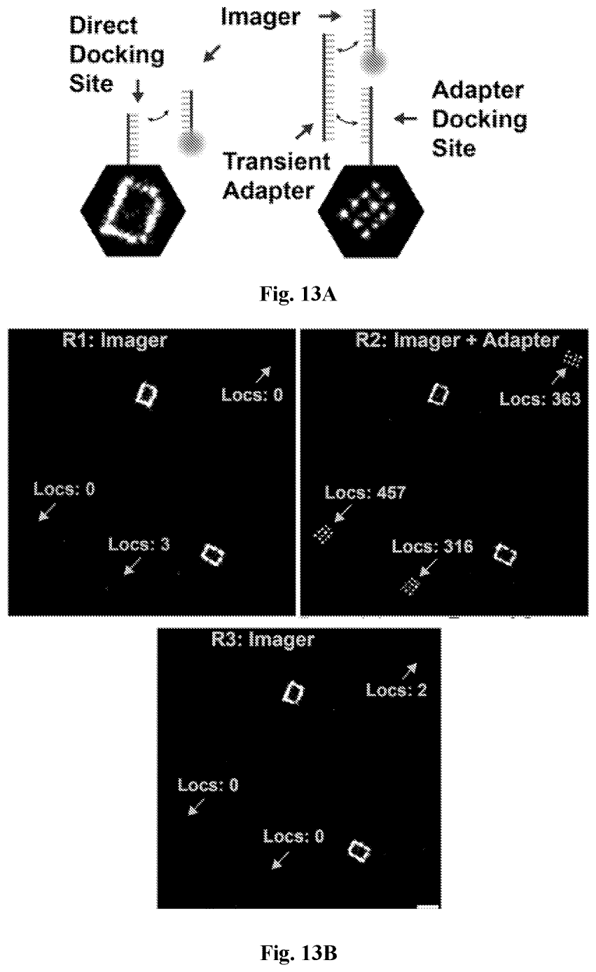

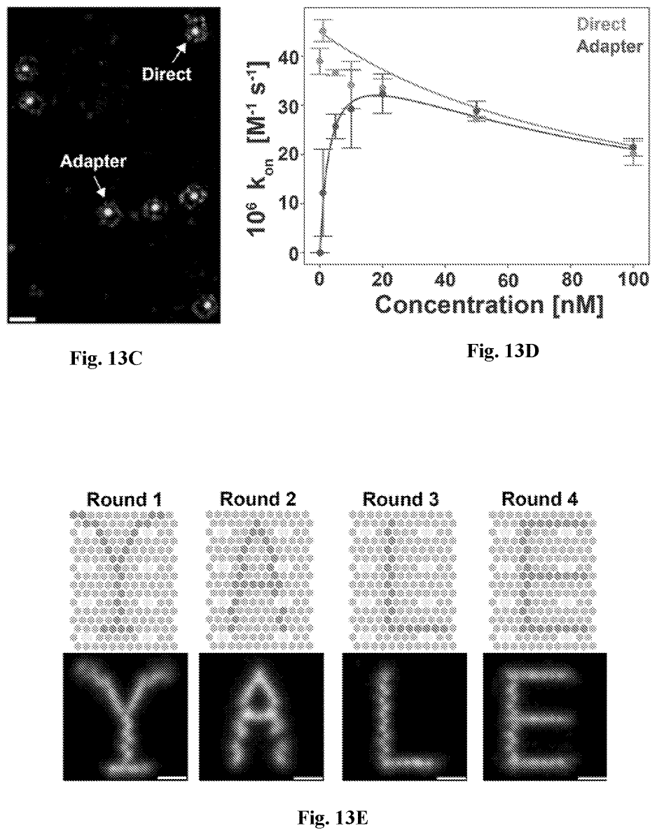

FIGS. 21A-21C: Three independent replicates of kinetics experiments measuring effective association rates of Imager probes binding directly or via Transient Adapters to DNA origami docking sites as a function of Transient Adapter concentration, in accordance with some embodiments. The A5-5×R2 Transient Adapter was used following the workflow described in FIGS. 19A-19I. The data points shown in FIG. 13D are the averaged values from these three experiments.

FIGS. 22A-22B: Measured dissociation rates for Transient Adapters for Imager R2 (speed Imager), in accordance with some embodiments. Dissociation rates for direct and Transient Adapter-mediated binding for all 12 Transient Adapters with the 5×R2 Imager docking site sequence using the workflow of FIGS. 19A-19I.

FIGS. 23A-23B: Measured association rates for Transient Adapters for Imager R2 (speed Imager), in accordance with some embodiments. Association rates for direct and Transient Adapter-mediated binding for all 12 Transient Adapters with the 5×R2 Imager docking site sequence using the workflow of FIGS. 19A-19I.

FIGS. 24A-24B: Measured dissociation rates for Transient Adapters for Imager P1 (classical Imager). Dissociation rates for direct and Transient Adapter-mediated binding for all 12 Transient Adapters with the P1 Imager docking site sequence using the workflow of FIGS. 19A-19I.

FIGS. 25A-25B: Measured association rates for Transient Adapters for Imager P1 (classical Imager), in accordance with some embodiments. Association rates for direct and Transient Adapter-mediated binding for all 12 Transient Adapters with the P1 Imager docking site sequence using the workflow of FIGS. 19A-19I.

FIGS. 26A-26B: Measured dissociation rates for Transient Adapters for Imager FP2 (fluorogenic Imager), in accordance with some embodiments. Dissociation rates for direct and

Transient Adapter-mediated binding for all 12 Transient Adapters with the FP2 Imager docking site sequence using the workflow of FIGS. 19A-19I.

FIGS. 27A-27B: Measured association rates for Transient Adapters for Imager FP2 (fluorogenic Imager), in accordance with some embodiments. Association rates for direct and Transient Adapter-mediated binding for all 12 Transient Adapters with the FP2 Imager docking site sequence using the workflow of FIGS. 19A-19I.

FIG. 28: Representative field of view of the 4-plex DNA origami letters experiment (FIG. 13E). Imaged DNA origami nano structures: Round 1: ‘Y’ & 20-nm grid & 10-nm grid (red); Round 2: ‘A’ & 20-nm grid & 10-nm grid (green), Round 3: ‘L’ & 20-nm grid & 10-nm grid (magenta); Round 4: ‘E’ & 20-nm grid & 10-nm grid (cyan). The 20-nm and 10-nm grids were imaged in each round and used for drift correction and alignment of the individual rounds. Scale bar 500 nm.

FIG. 29: Single DNA origami structures displaying the letter ‘Y’, in accordance with some embodiments. Images of the 246 single structures used to produce the averaged image depicted in FIG. 13E. Scale bar 100 nm.

FIG. 30: Single DNA origami structures displaying the letter ‘A’, in accordance with some embodiment. Images of 224 single structures used to produce the averaged image depicted in FIG. 13E. Scale bar 100 nm

FIG. 31: Single DNA origami structures displaying the letter ‘L’, in accordance with some embodiment. Images of 121 single structures used to produce the averaged image depicted in FIG. 13E. Scale bar 100 nm.

FIG. 32: Single DNA origami structures displaying the letter ‘E’, in accordance with some embodiments. Images of 191 single structures used to produce the averaged image depicted in FIG. 13E. Scale bar 100 nm.

FIGS. 33A-33B: Direct comparison of imaging performance for fluorogenic, speed, and classical Imagers, in accordance with some embodiments. Mitochondria were immunolabeled with a primary antibody against Tom20. Secondary antibodies were conjugated with a docking site for the A3 Transient Adapter. In the first round of the sequential imaging experiment, the fluorogenic Imager FP2 and a Transient Adapter to FP2 were used. After data acquisition, both were washed out and replaced by the speed Imager R2 and a Transient Adapter with a 5×R2 motif. After a second round of data acquisition the Imager and Transient Adapter were washed out once more and replaced by the classical Imager P1 and a corresponding Transient Adapter. A third round of data acquisition was followed by another round of washes and reintroduction of fluorogenic Imager FP2 and a Transient Adapter to FP2 to verify that sample degradation was negligible. A comparison shows a substantial improvement of the signal-to-background ratio using the fluorogenic and the speed Imagers (SNR≈40) over the classical Imager P1 (SNR≈8). Scale bar 2 μm.

FIGS. 34A-34B: Quantification of erasing efficiency for all 12 Transient Adapter sequences, in accordance with some embodiments. A modified workflow of FIGS. 19A-19I was used: in the first round, only the Imager but no Transient Adapter is introduced. In the second round, both the Transient Adapter (20 nM) and Imager are used. During the final round of imaging, the solution is replaced (no washes) by the corresponding Eraser strand (100 nM) and the same Imager. After waiting for 3 min, the third round of imaging is conducted. The analyzed single docking sites are divided into ten random groups, from which the mean and standard deviation are calculated. All values are normalized.

FIGS. 35A-35B: Observation of molecular target switch dynamics, in accordance with some embodiments. FIG. 35A: U-2 OS cell labeled with antibodies against mitochondrial outer membrane protein Tom20 and α-tubulin. Before the shown time course, the sample was in a medium containing a Transient Adapter directing Imager R2 to mitochondria. At the start of image acquisition, a corresponding Eraser and a new Transient Adapter which directs the Imager to α-tubulin are added. The Eraser rapidly erases the mitochondria signal (τ_{1/2}≈60 s) as the α-tubulin signal, mediated by the new Transient Adapter, increases (τ_{1/2}≈200 s). FIG. 35B: Equivalent experiment using antibodies against NPM1 (nucleolus) and LaminB1 (nuclear envelope). Data was acquired with a spinning disk microscope and is diffraction limited. Scale bar 5 μm.

FIGS. 36A-36D: Evaluation of non-specific binding of the Transient Adapter and speed Imager system, in accordance with some embodiments. The mitochondrial protein Tom20 in U-2 OS cells is immunolabeled with a A5 docking site using primary and secondary antibodies. FIG. 36A: In the first round, to identify a field of view, the classical P1 Imager and the Transient Adapter directing P1 to A5 are introduced. The histogram displays a one-dimensional projection of localizations along the arrows at the highlighted region of interest and shows robust signal. FIG. 36B: After two brief washes, the R2 Imager is introduced. Since the corresponding Transient Adapter is absent, no specific binding is expected, and the corresponding histogram shows very few localization events. FIG. 36C: In the third round of imaging, all Transient Adapters except for the correct one (A5-5×R2) are added to the R2 Imager. As none of the introduced Transient Adapters should be able to interact with the docking site, no specific interaction or downstream sampling is anticipated. This is confirmed by the histogram which again shows only very few localization events. FIG. 36D: In the fourth round of imaging, the matching Transient Adapter (A5-5×R2) is introduced alongside the Imager R2. As expected, the histogram shows robust signal, comparable to (a). Scale bar 2 μm.

FIGS. 37A-37D: Evaluation of non-specific binding of the Transient Adapter and classical Imager system, in accordance with some embodiments. The mitochondrial protein Tom20 in U-2 OS cells is immunolabeled with a A5 docking site using primary and secondary antibodies. FIG. 37A: In the first round, to identify a field of view, the R2 speed Imager and the Transient Adapter directing R2 to A5 are introduced. The histogram displays a one-dimensional projection of localizations along the arrows at the highlighted region of interest and shows robust signal. FIG. 37B: After two brief washes, the P1 Imager is introduced. Since the corresponding Transient Adapter is absent, no specific binding is expected, and the corresponding histogram shows very few localization events. FIG. 37C: In the third round of imaging, all Transient Adapters except for the correct one (A5-P1) are added to the P1 Imager. As none of the introduced Transient Adapters should be able to interact with the docking site, no specific interaction or downstream sampling is anticipated. This is confirmed by the histogram which again shows only very few localization events. FIG. 37D: In the fourth round of imaging, the matching Transient Adapter (A5-P1) is introduced alongside the Imager P1. As expected, the histogram shows robust signal, comparable to FIG. 37A. Scale bar 2 μm.

FIGS. 38A-38D: Evaluation of non-specific binding of the Transient Adapter and fluorogenic Imager system, in accordance with some embodiments. The mitochondrial protein Tom20 in U-2 OS cells is immunolabeled with a A5 docking site using primary and secondary antibodies. FIG. 38A: In the first round, to identify a field of view, the classical P1 Imager and the Transient Adapter directing P1 to A5 are introduced. The histogram displays a one-dimensional projection of localizations along the arrows at the highlighted region of interest and shows robust signal. FIG. 38B: After two brief washes, the FP2 Imager is introduced. Since the corresponding Transient Adapter is absent, no specific binding is expected, and the corresponding histogram shows very few localization events. FIG. 38C: In the third round of imaging, all Transient Adapters except for the correct one (A5-FP2) are added to the FP2 Imager. As none of the introduced Transient Adapters should be able to interact with the docking site, no specific interaction or downstream sampling is anticipated. This is confirmed by the histogram which again shows only very few localization events. FIG. 38D: In the fourth round of imaging, the matching Transient Adapter (A5-FP2) is introduced alongside the Imager FP2. As expected, the histogram shows robust signal, comparable to FIG. 38A. Scale bar 2 μm.

FIG. 39: Images of the individual imaging rounds of FIGS. 21A-21C, in accordance with some embodiments. Additionally, to the single-target imaging rounds, for alignment purposes, the sample is imaged an additional time with all targets simultaneously. Scale bars: 5 μm.

FIGS. 40A-40O: Cilia-targeted antibody Light-Activated Site-specific Conjugation (LASIC) and images of the individual imaging rounds of FIGS. 22A-22B with image segmentation, in accordance with some embodiments. FIG. 40A: Coomassie blue-stained SDS-PAGE gel showing the direct conjugation of seven distinct OyOlink binder probe sequences (Alpha Thera) to cilia-specific antibodies. Conjugated heavy chain IgG transitions from 60 kDa (− lanes) to 75 kDa (+ lanes) upon incubation with OyOlink probe for 2 h under 365-nm UV light.

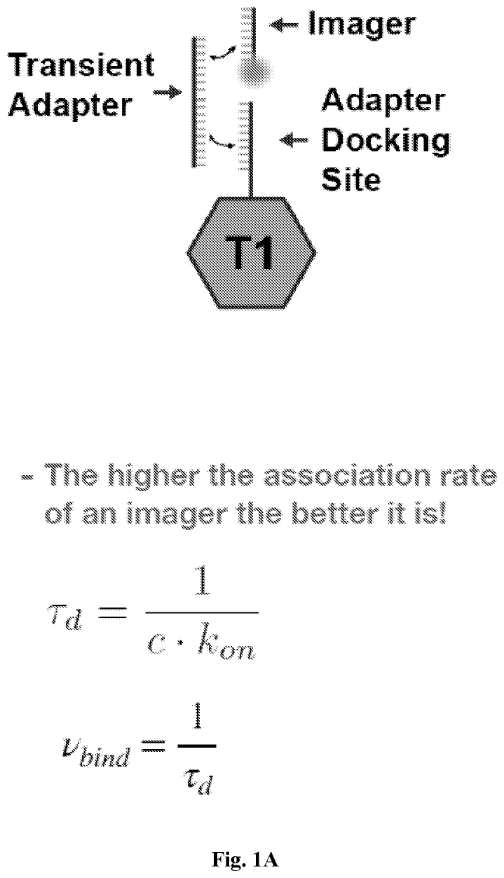

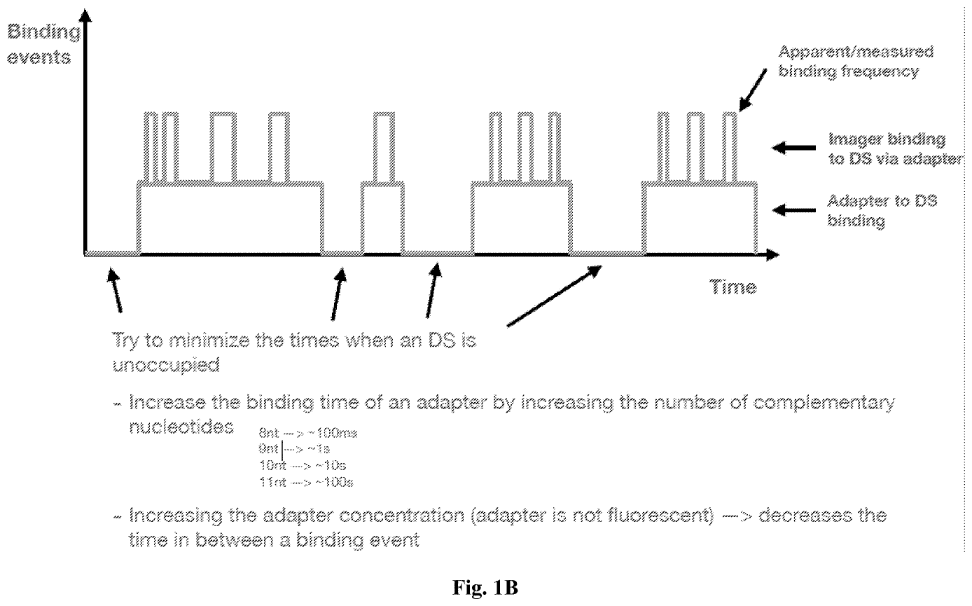

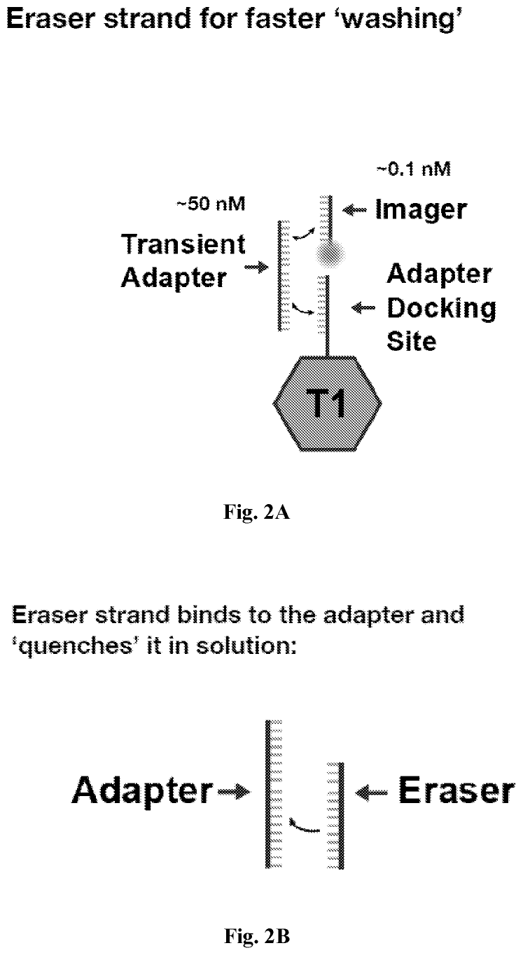

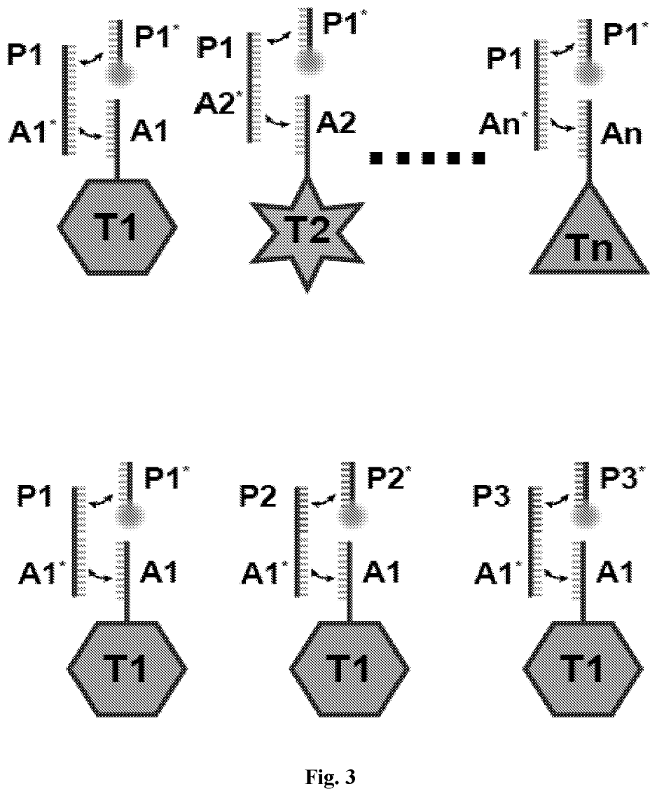

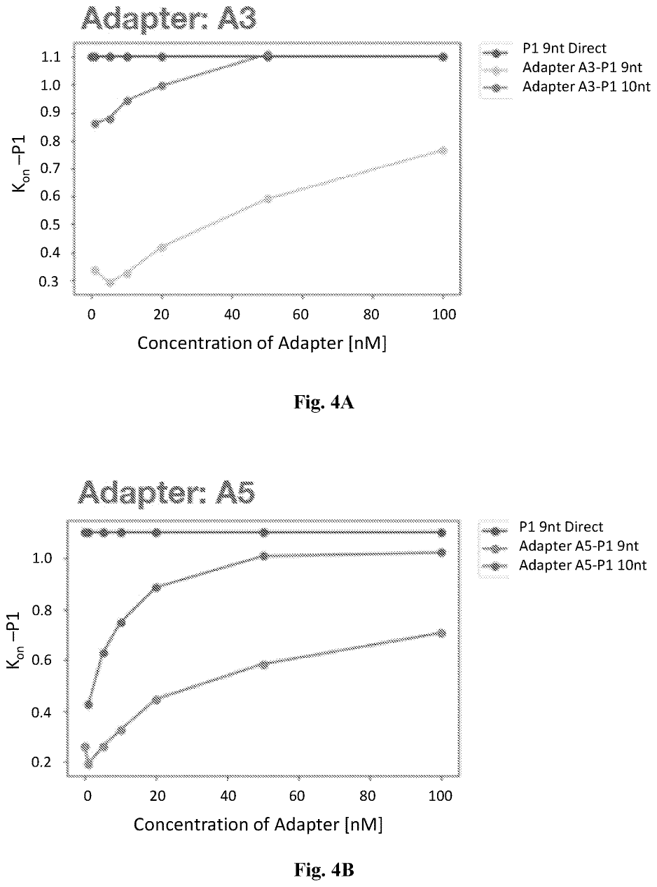

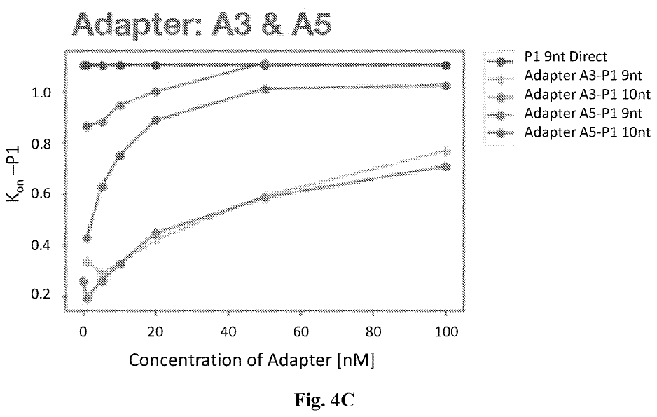

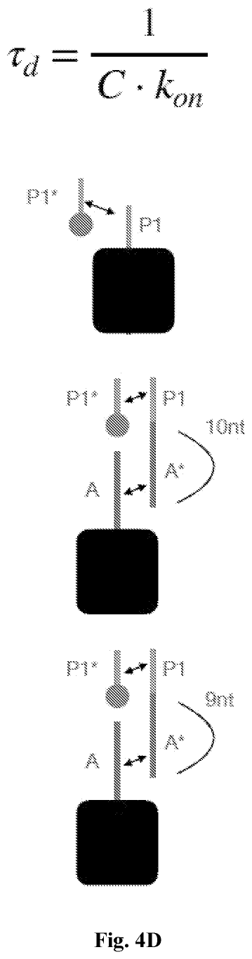

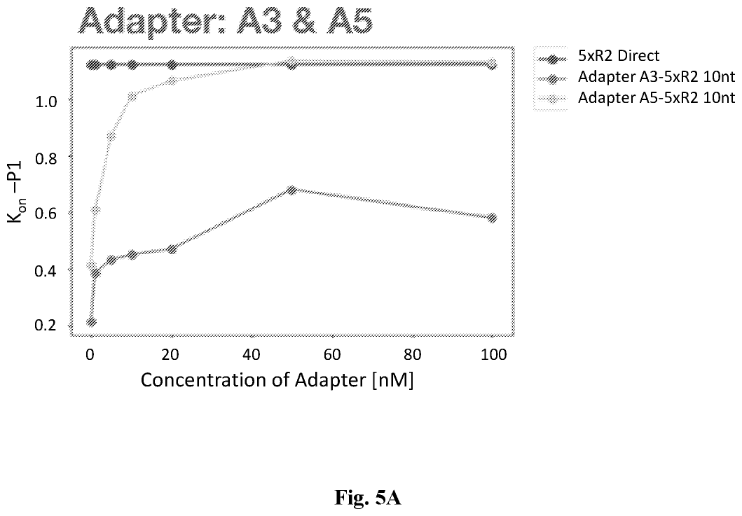

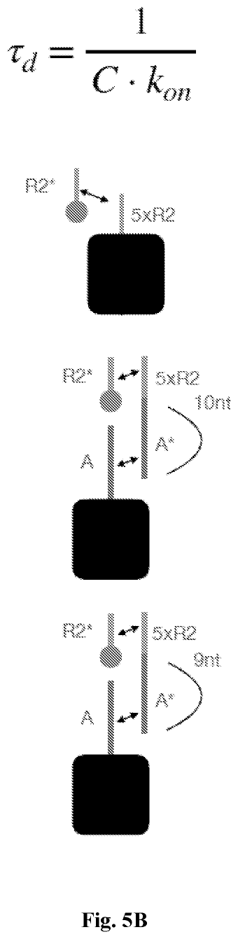

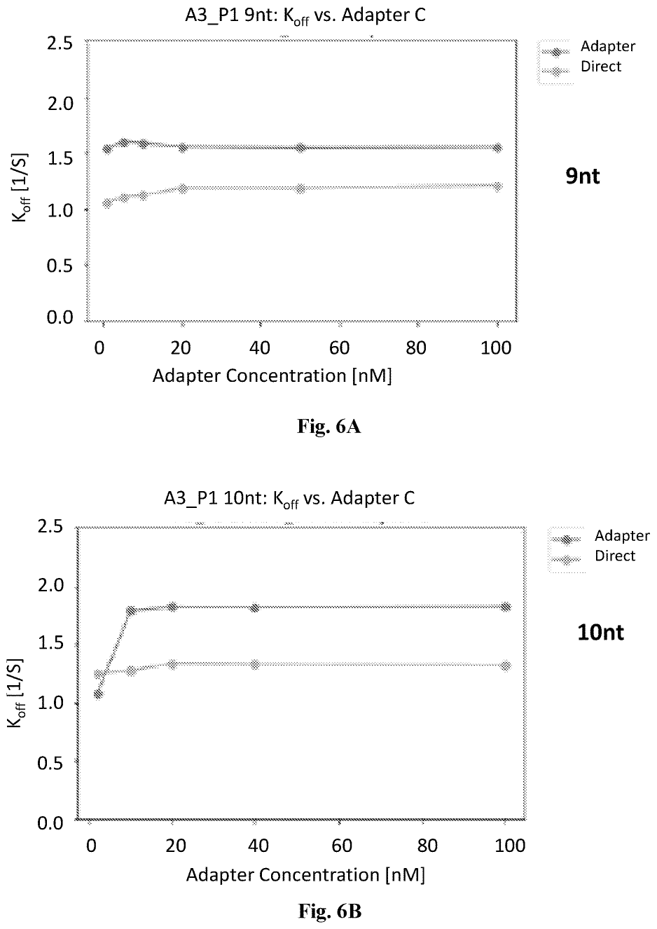

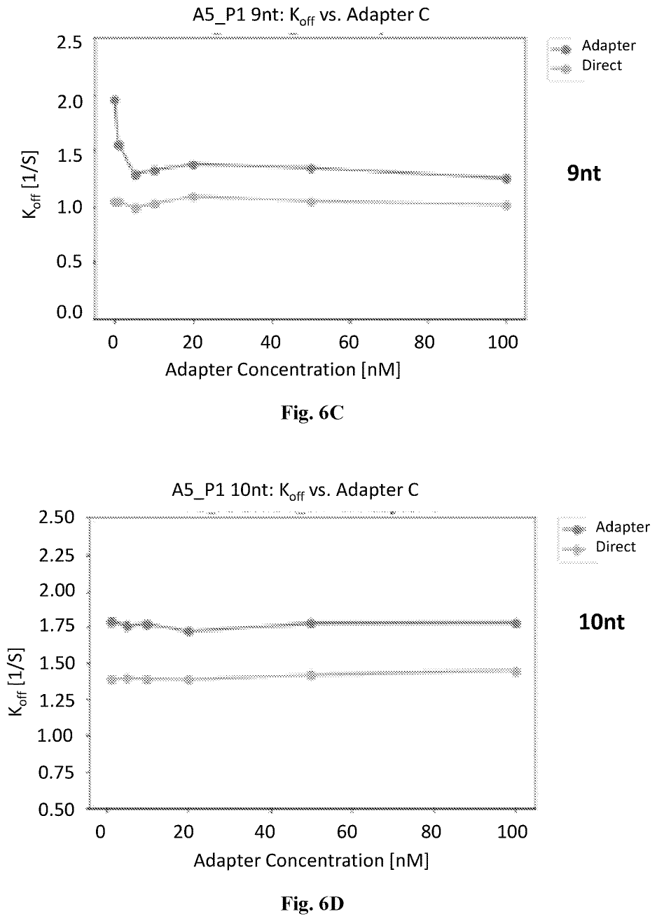

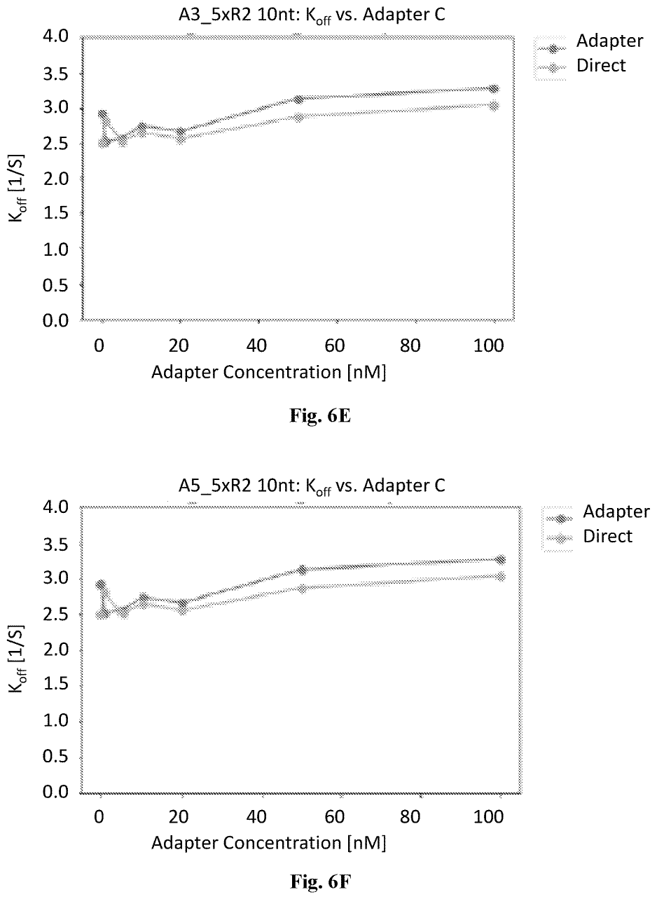

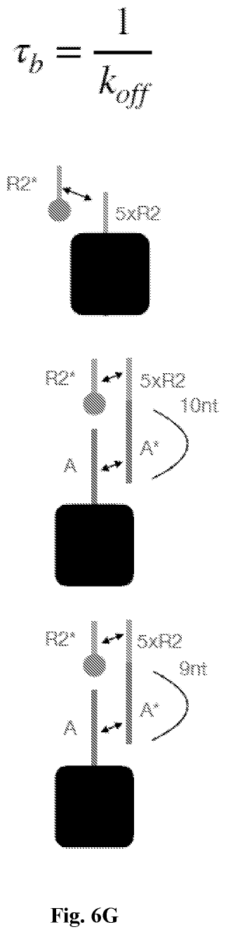

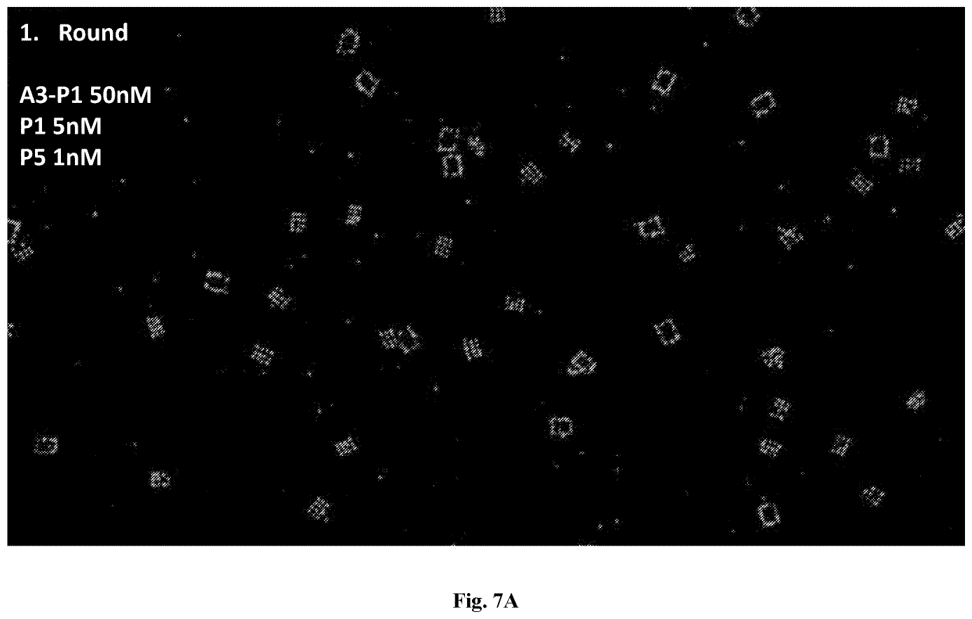

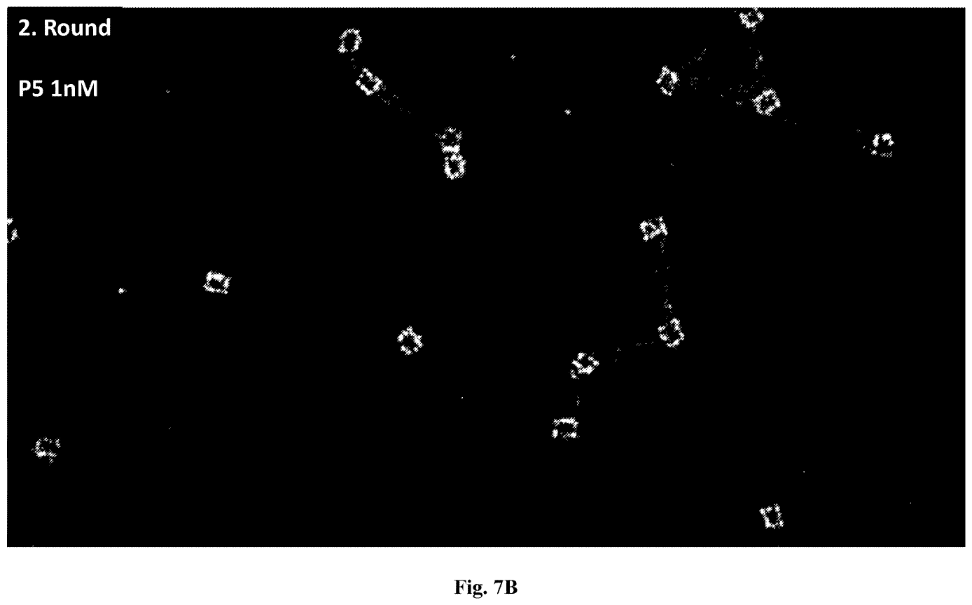

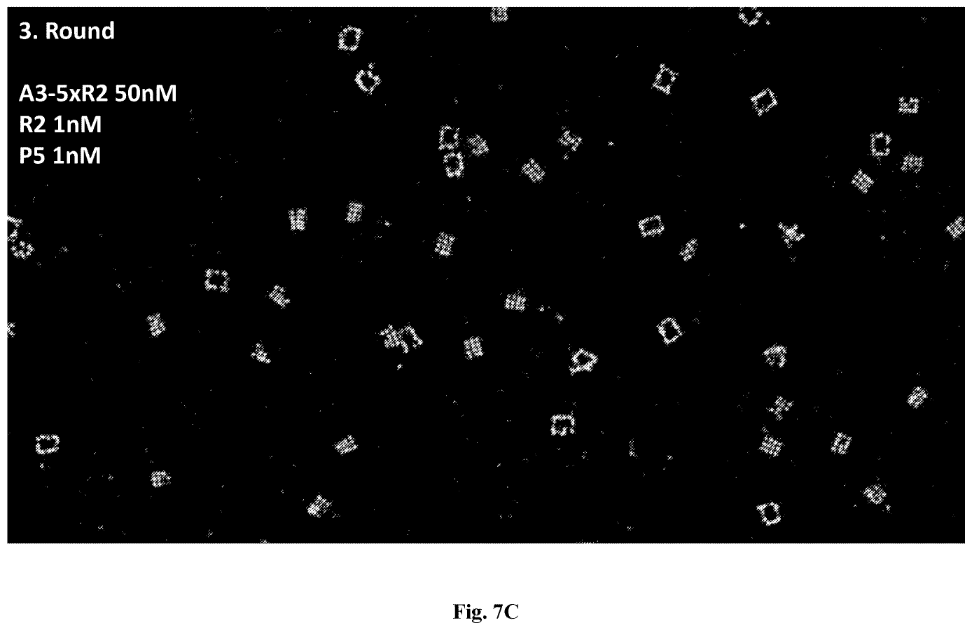

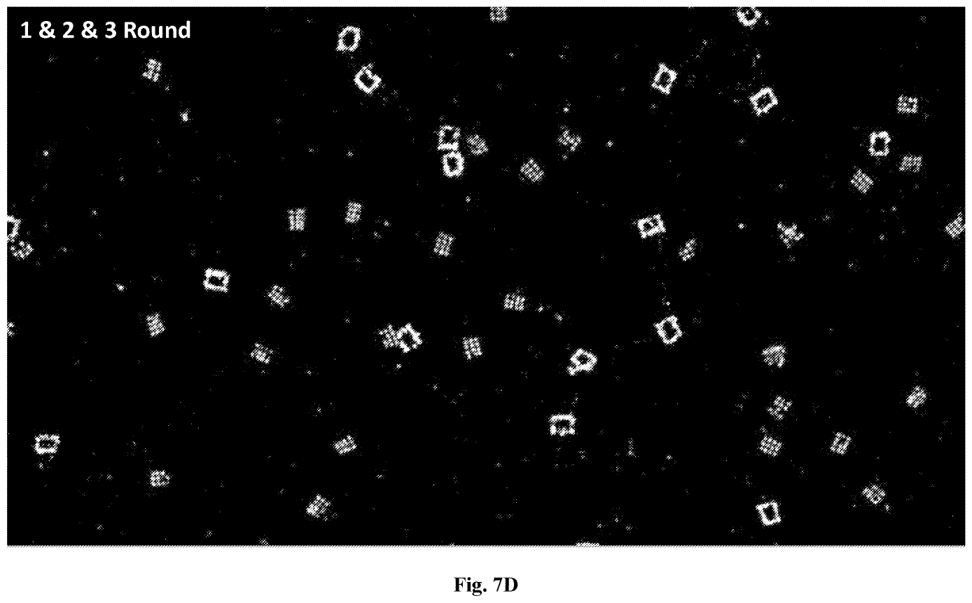

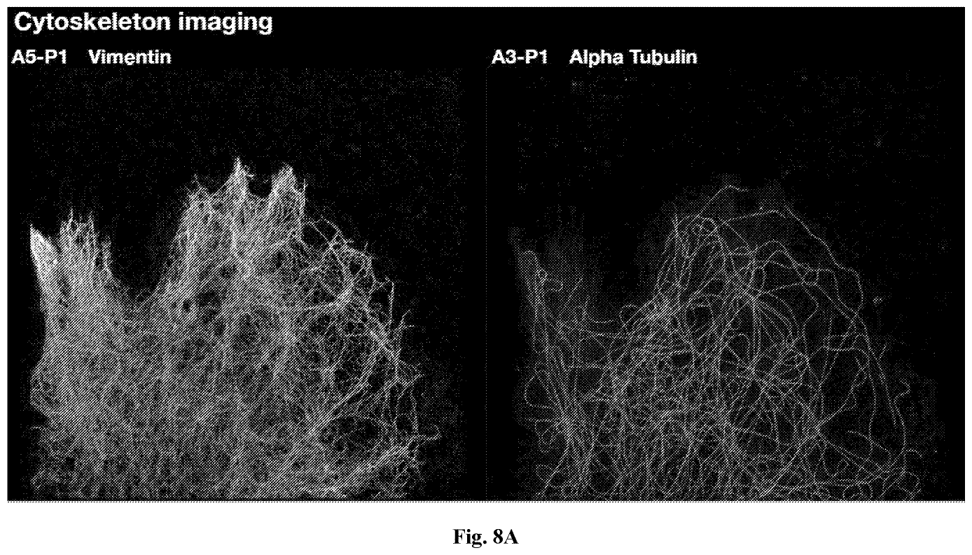

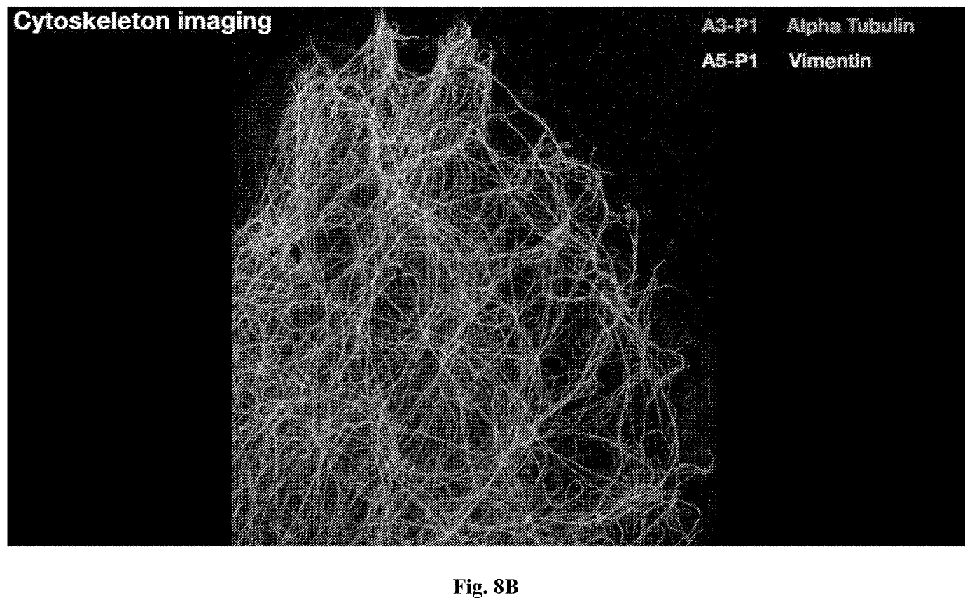

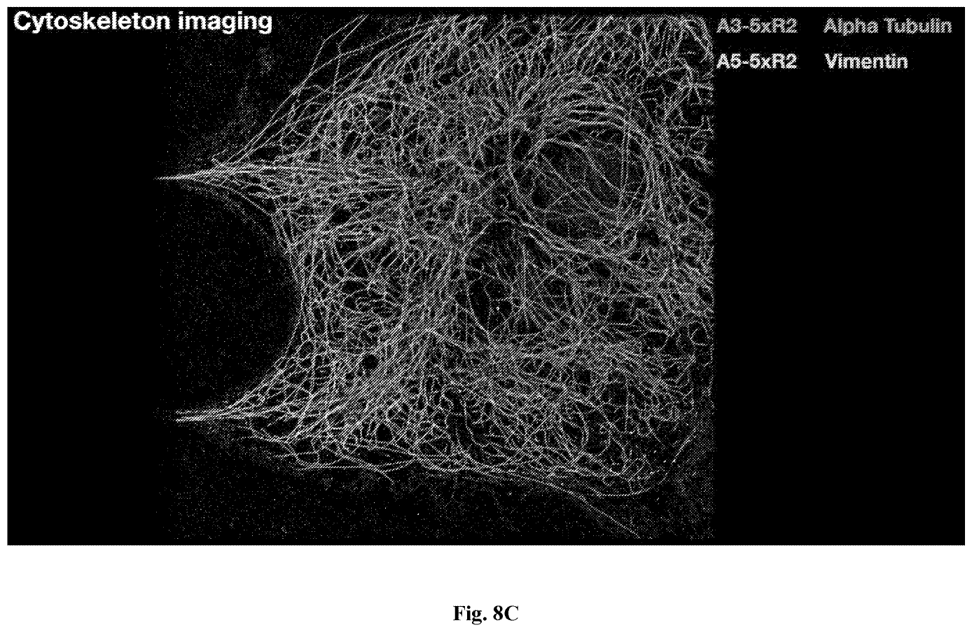

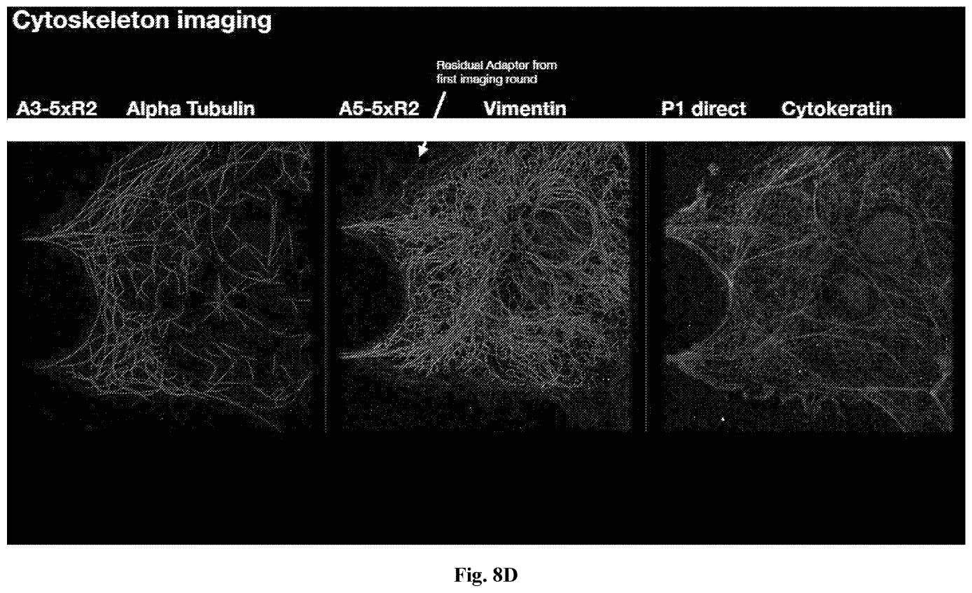

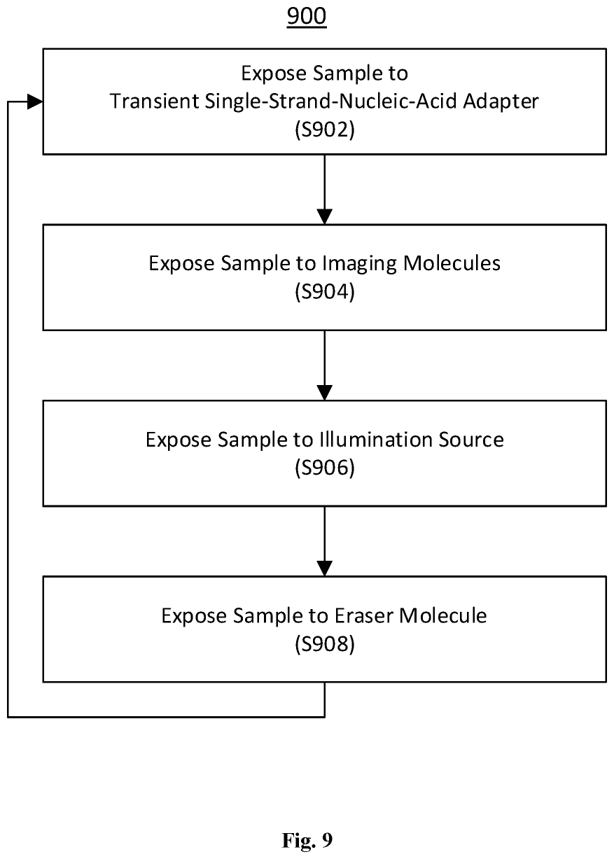

FIGS. 40B-40J: Images of the individual imaging rounds of FIGS. 22A-22B. FIG. 40K: z-projection of the pH-Smo data, illustrating the full 1.5-μm z-range. FIGS. 40I-40M: Zoomed-in views of the two analyzed cilia. FIGS. 40N-40O: Examples of surface, clusters, and filaments generated with Imaris software for the pHSmo and ac-tub targets. Scale bars: 5 μm (FIGS. 40B-40K), 1 μm (FIGS. 40L-40O).

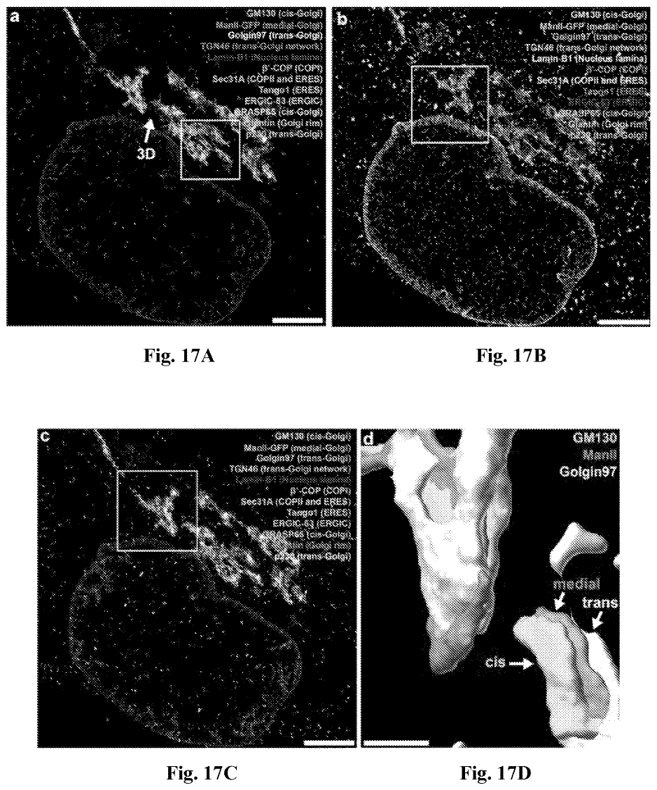

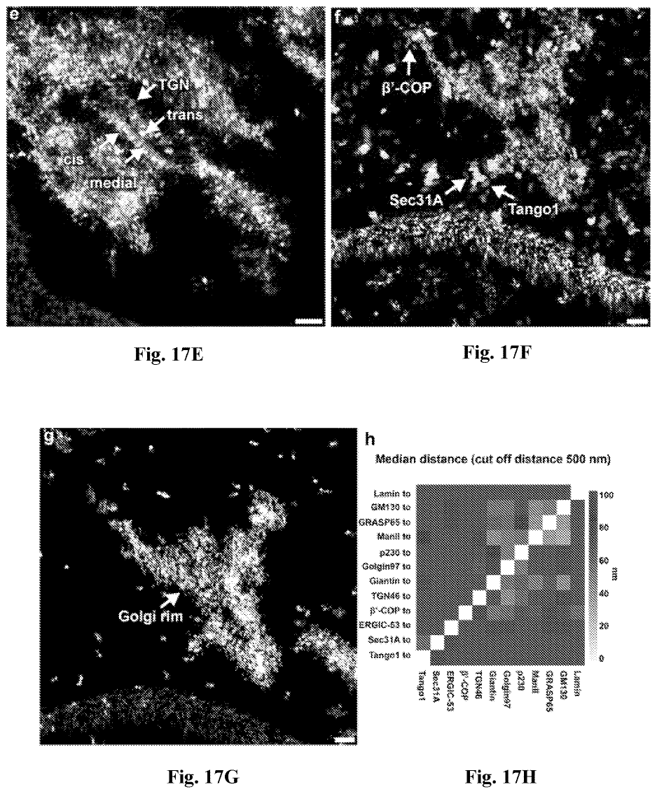

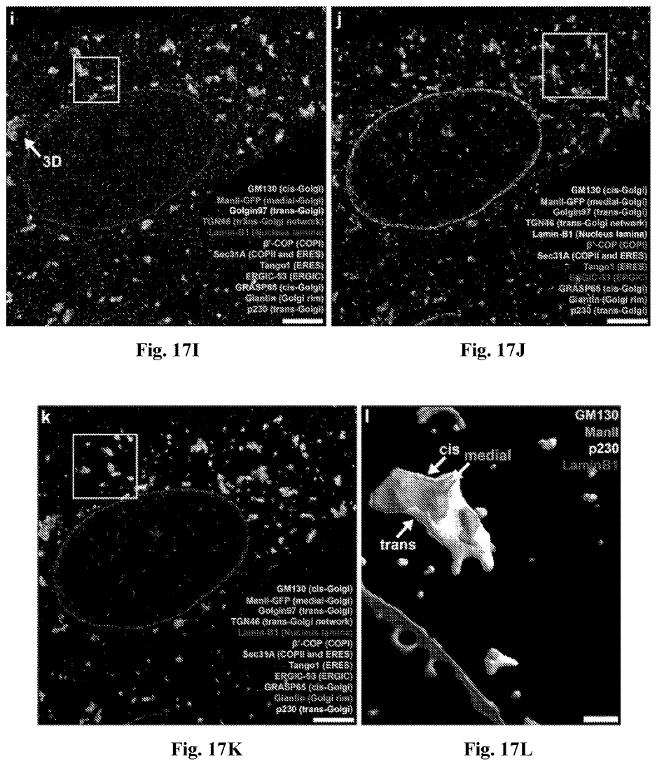

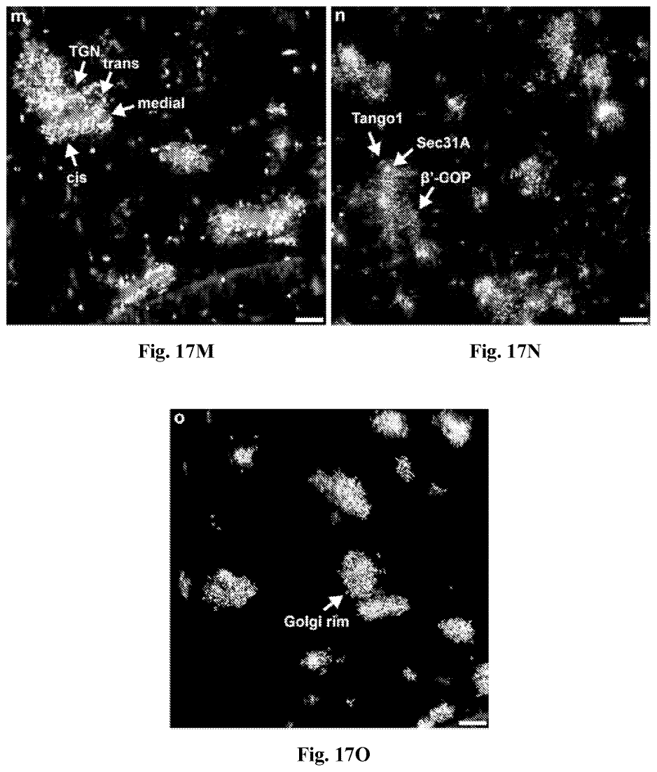

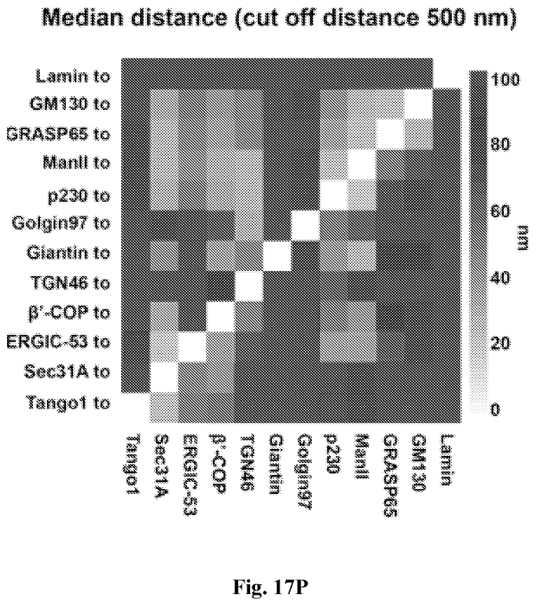

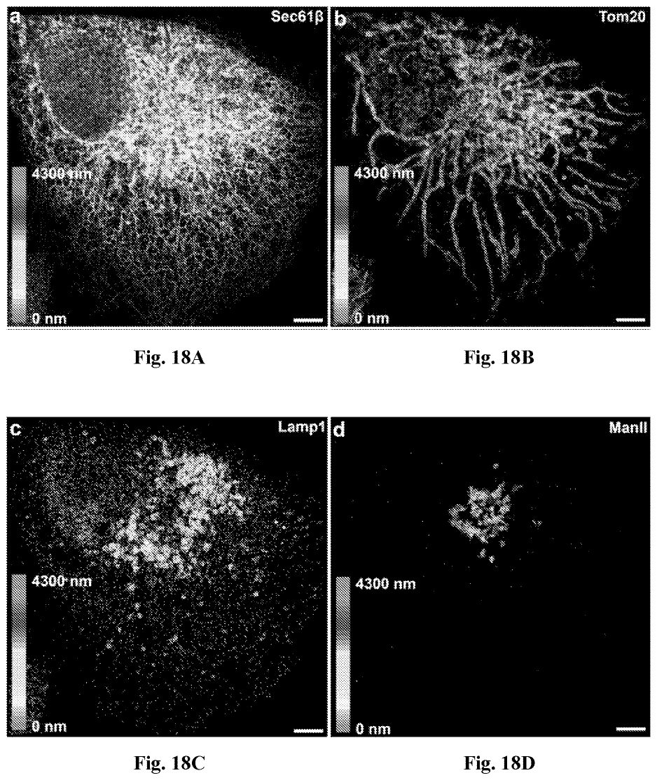

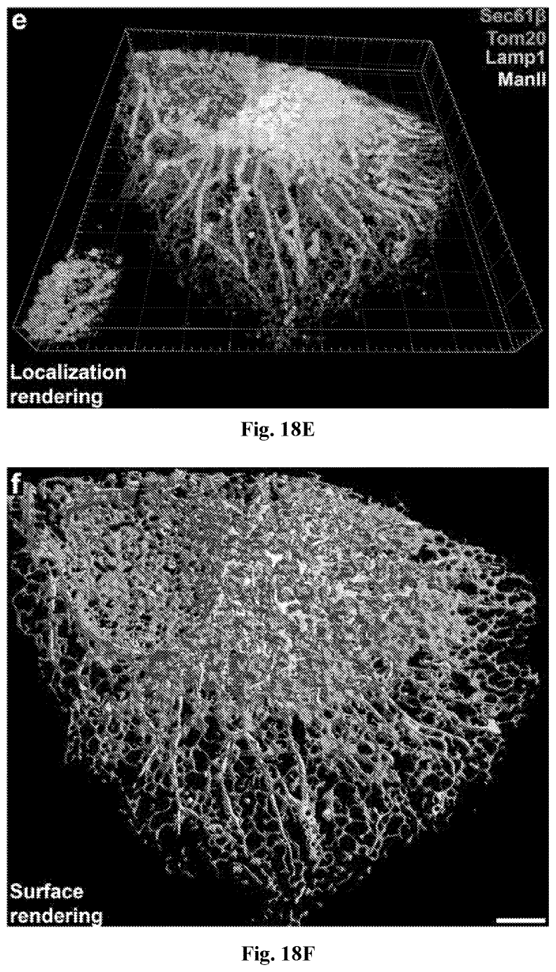

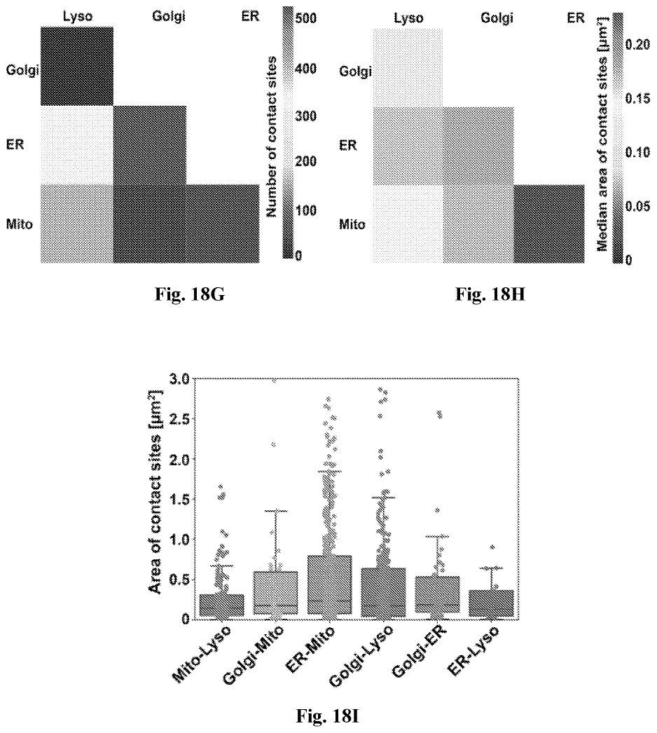

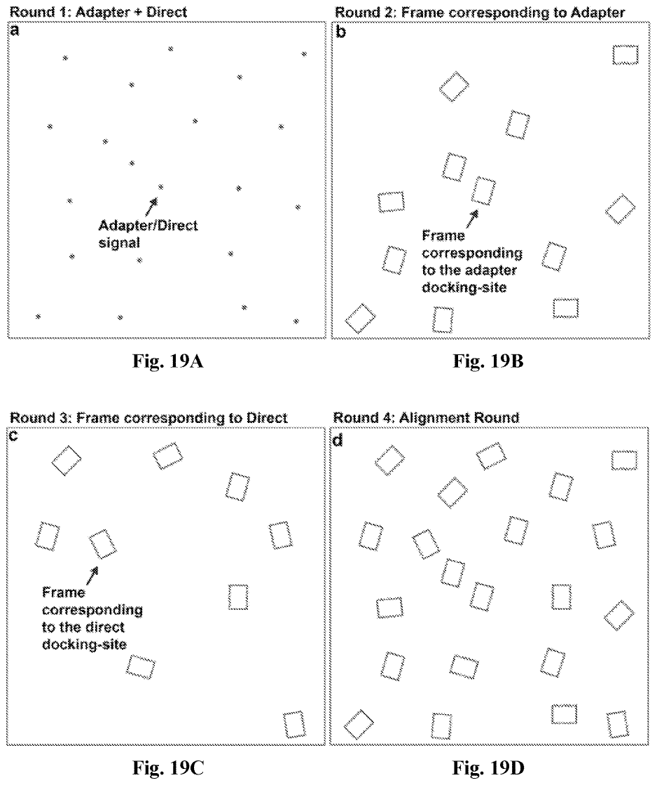

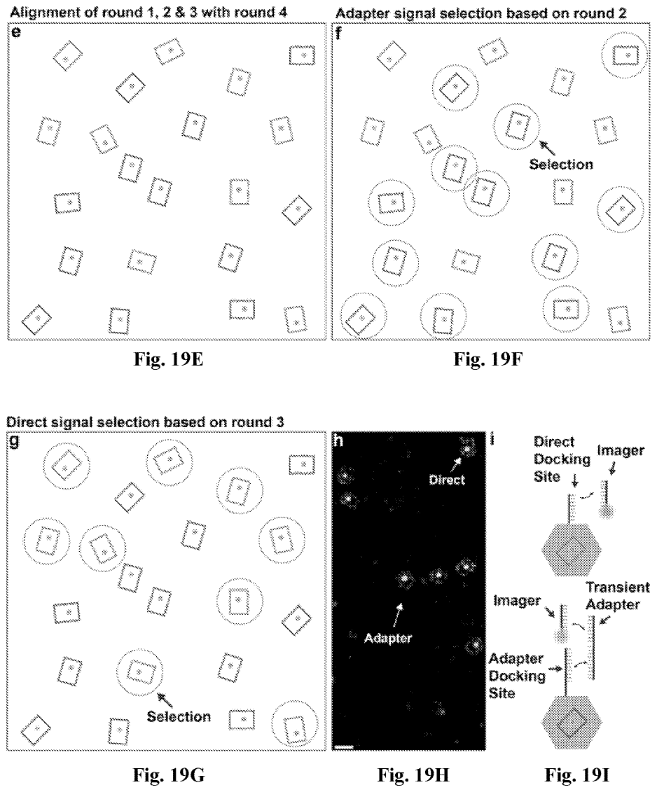

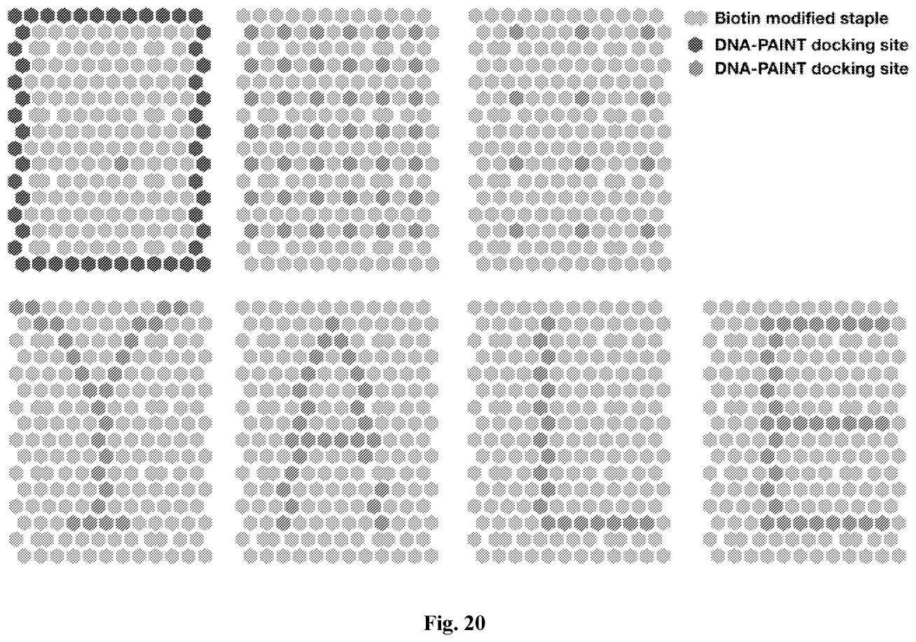

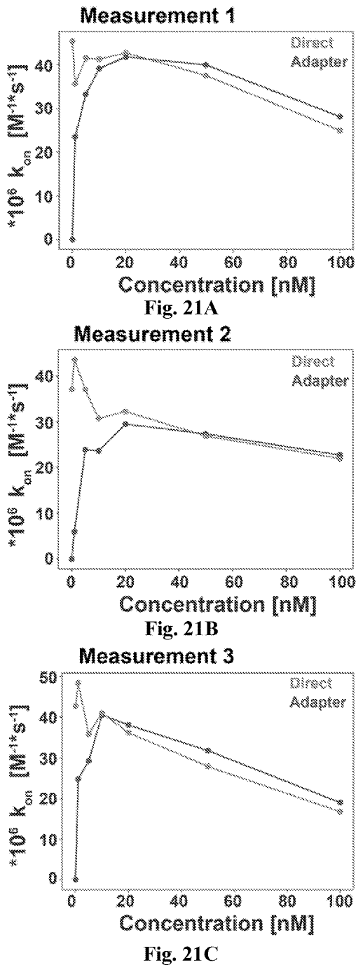

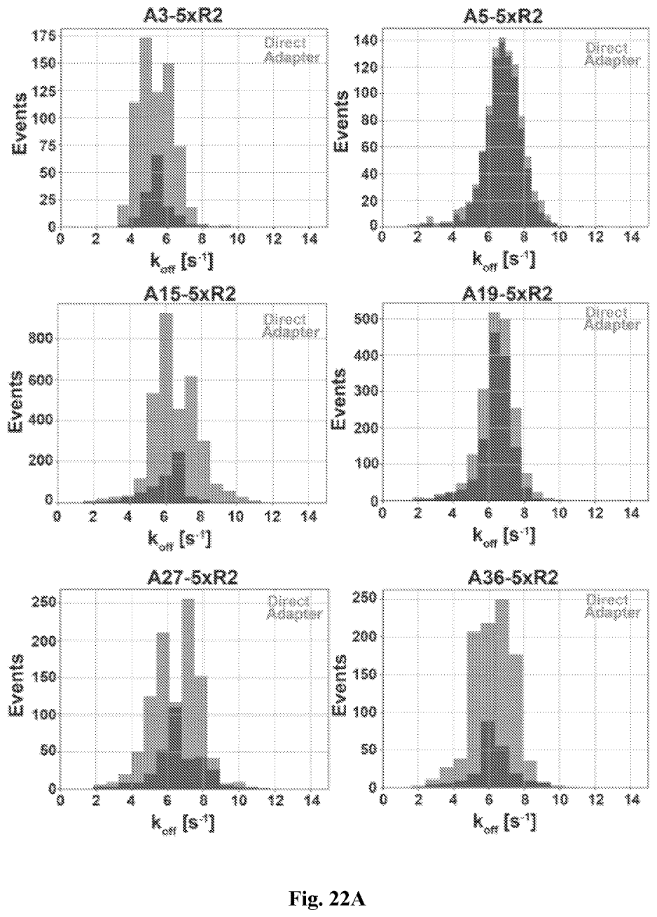

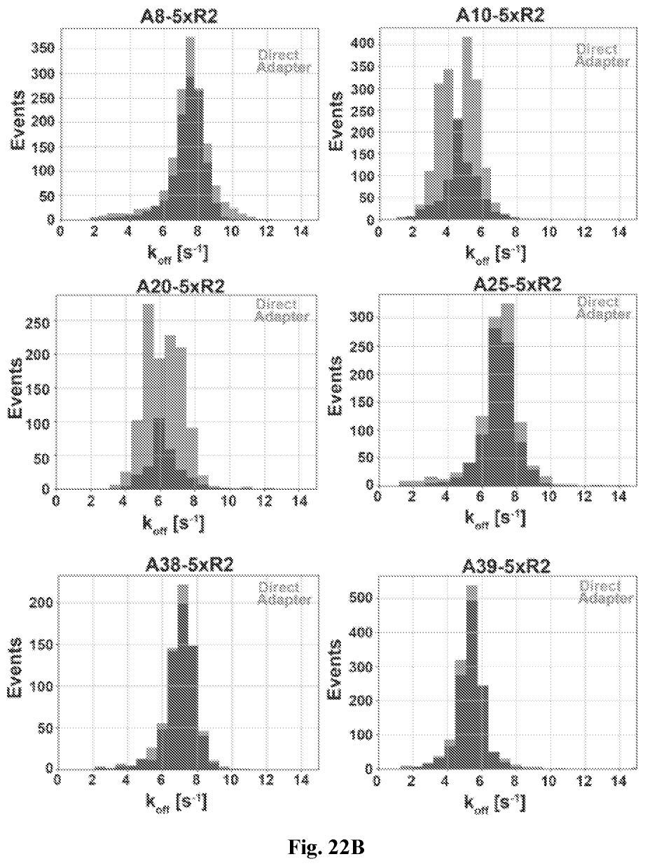

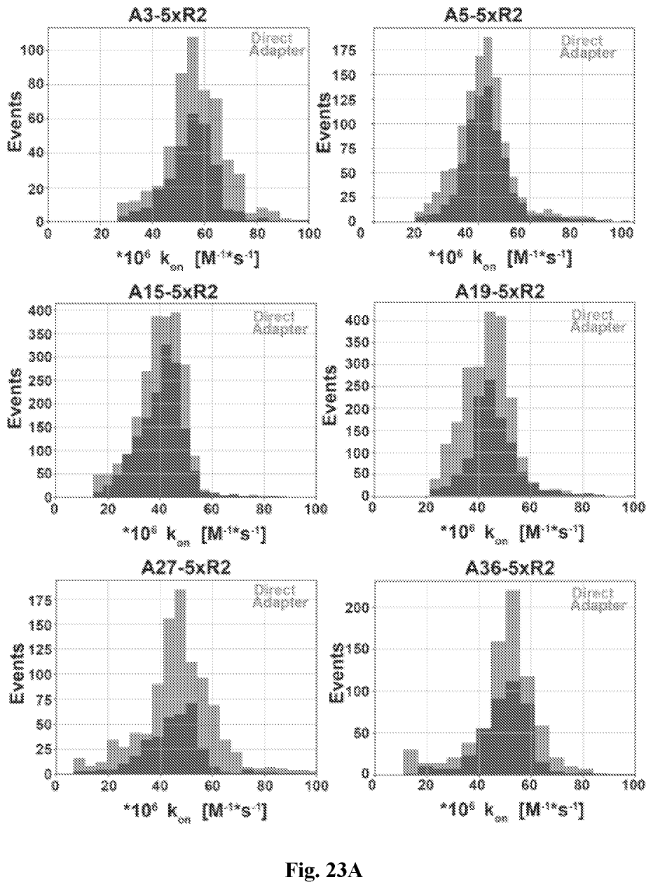

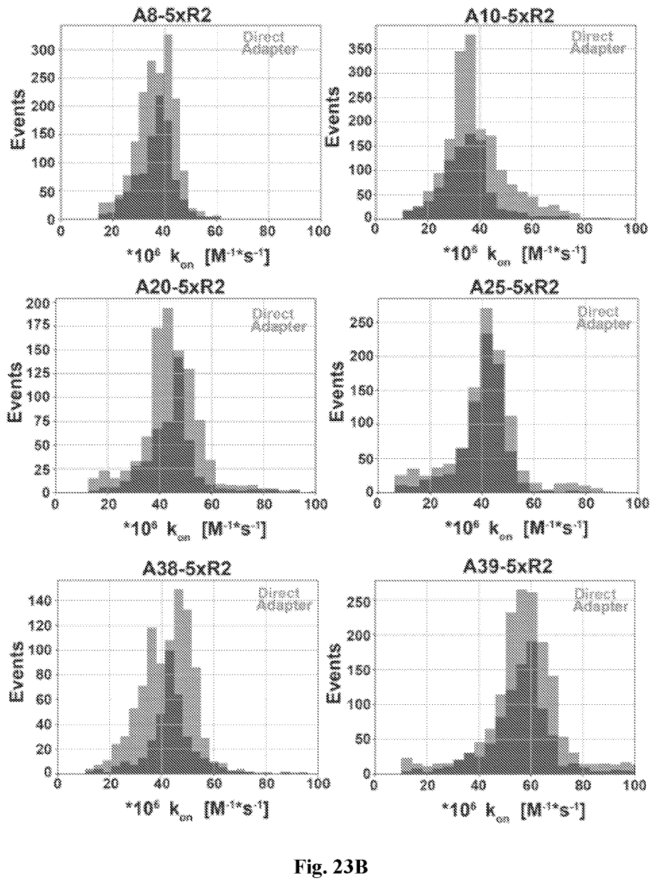

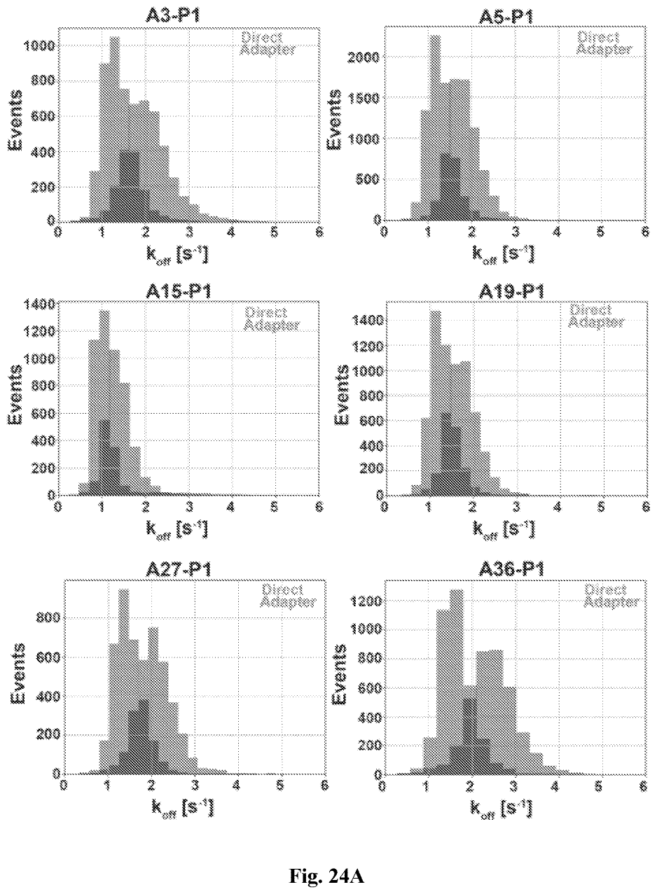

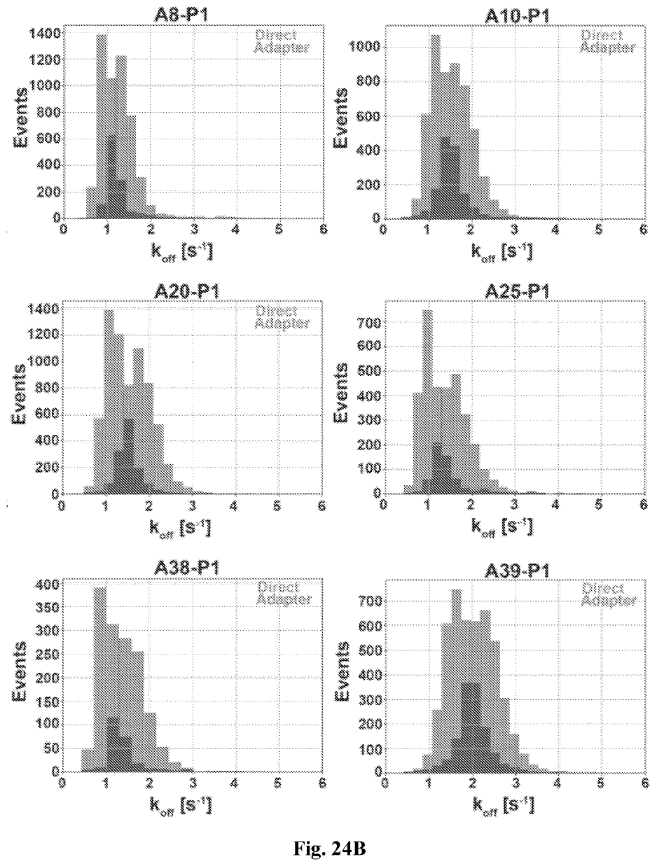

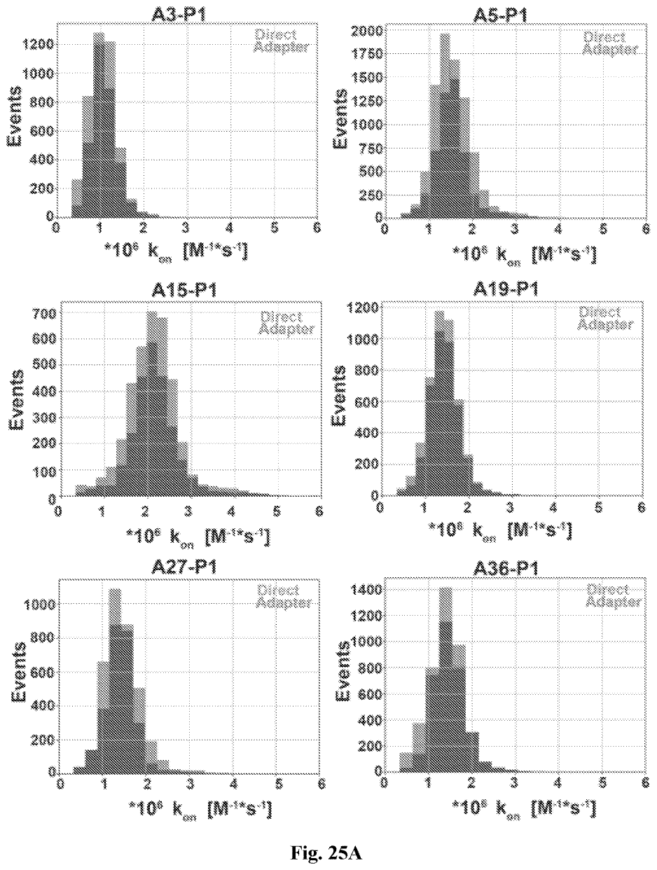

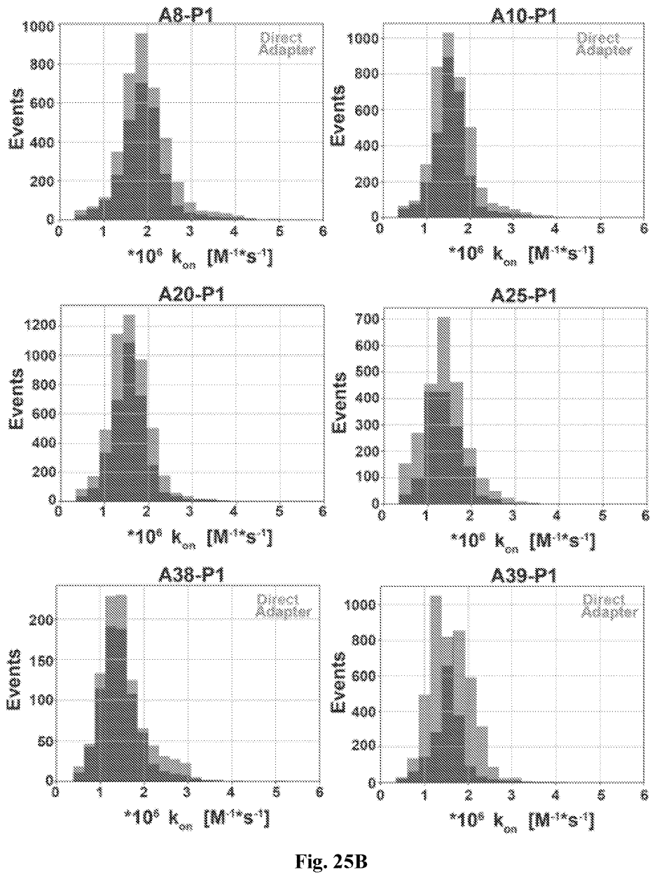

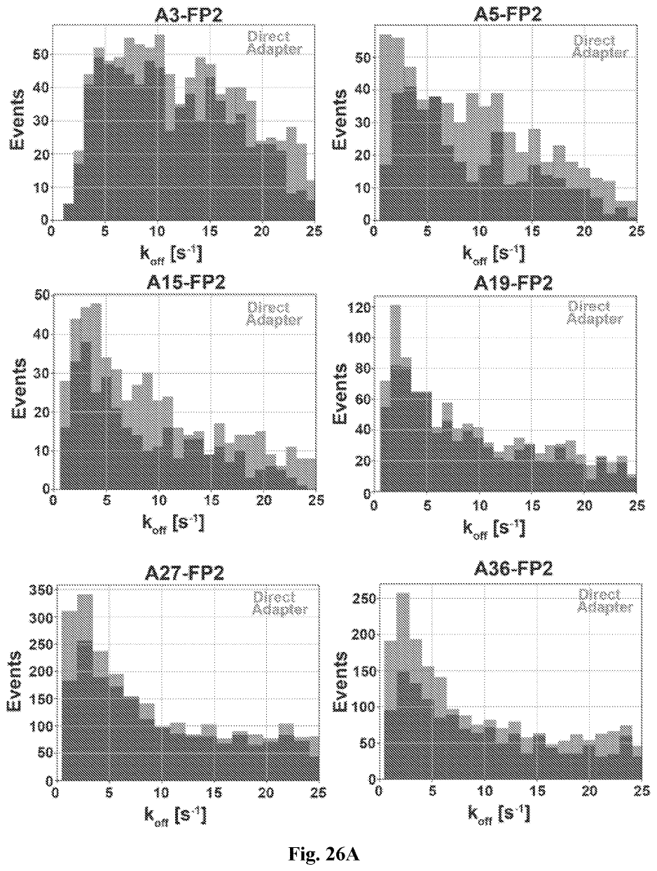

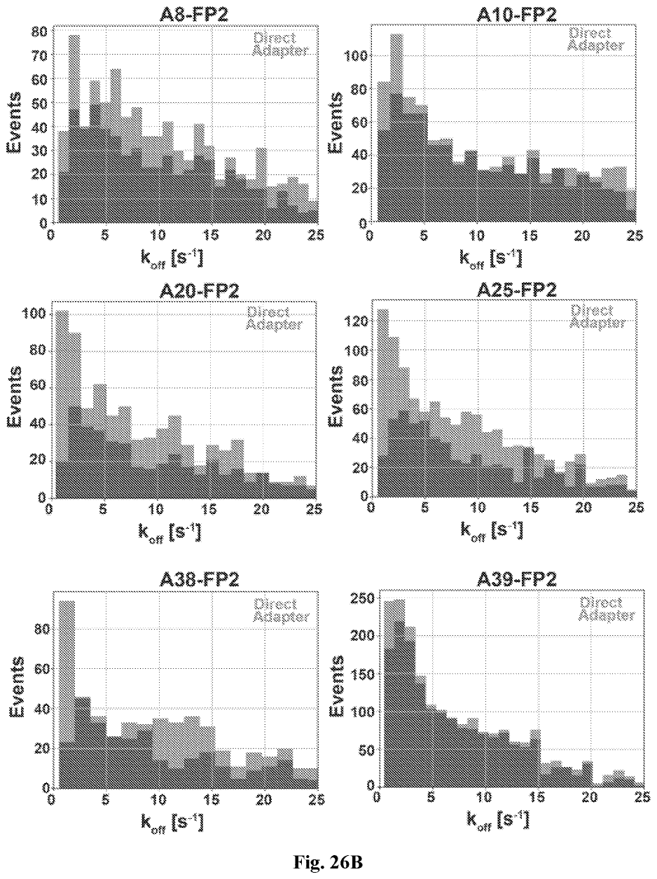

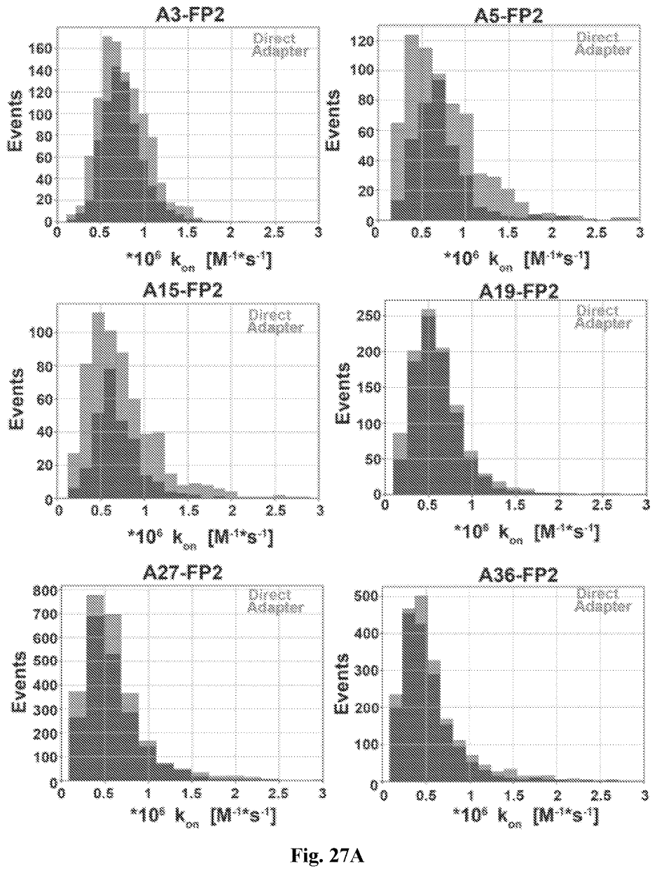

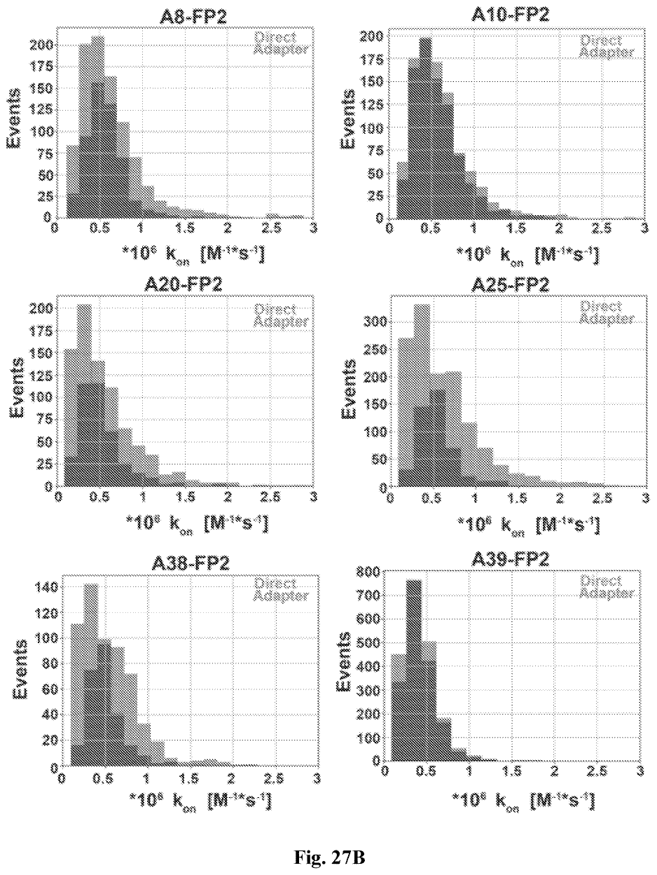

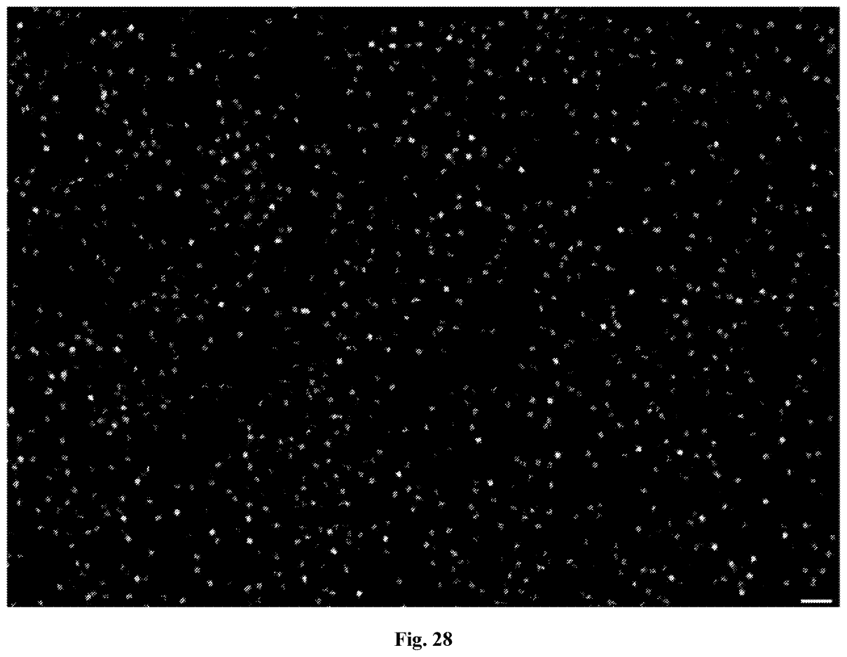

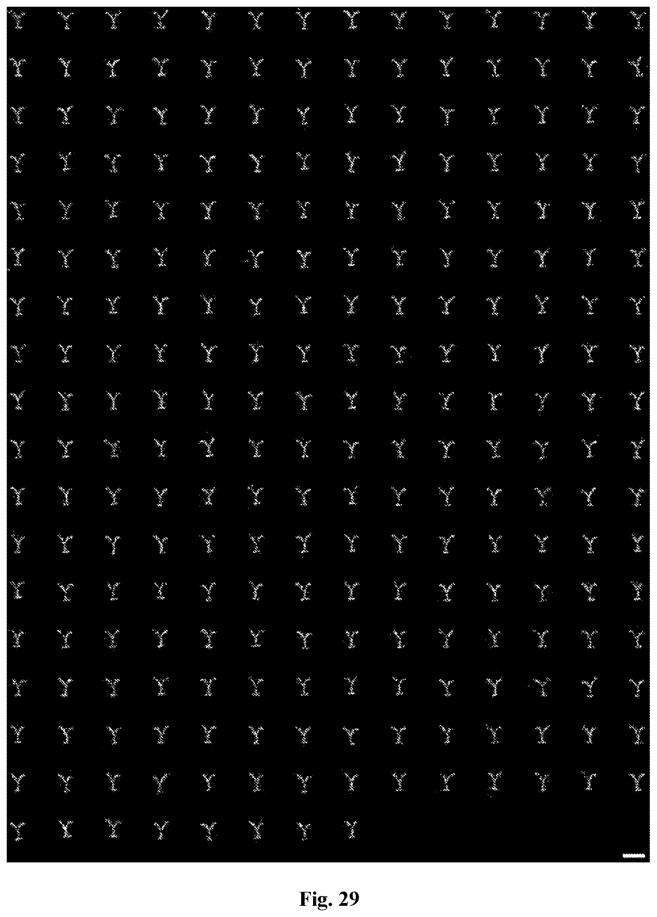

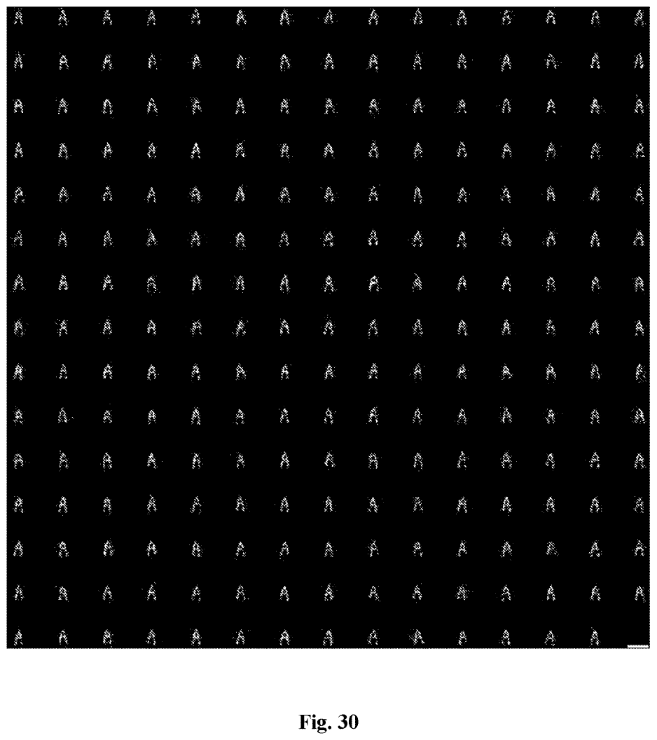

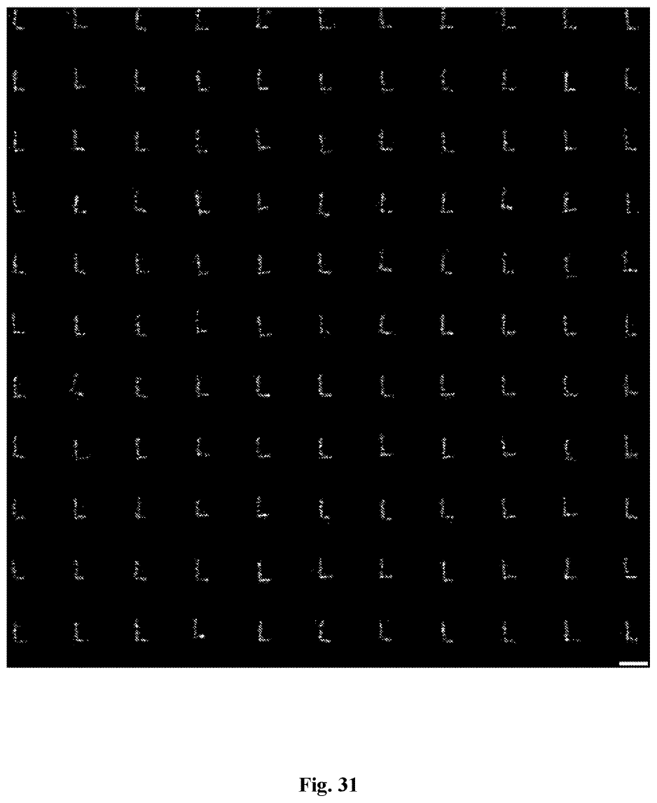

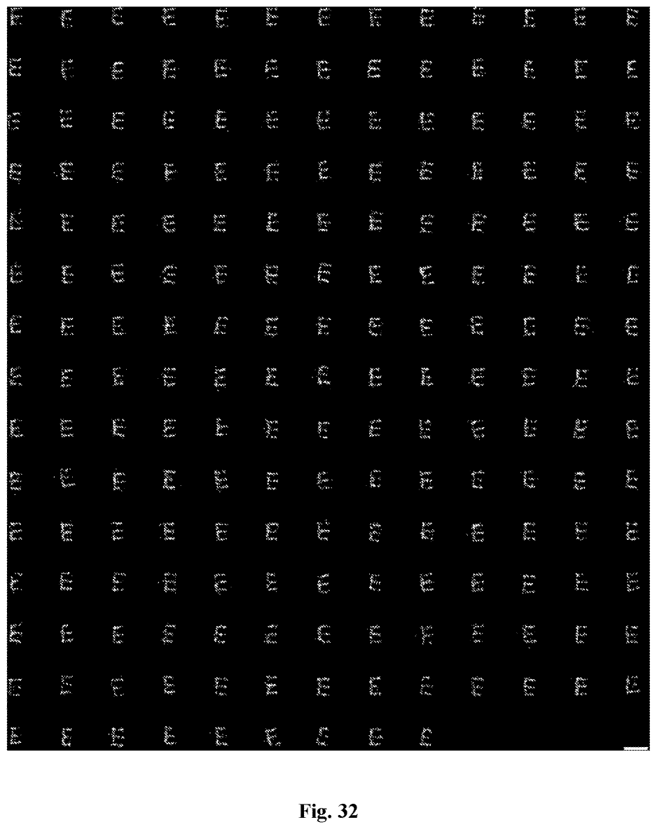

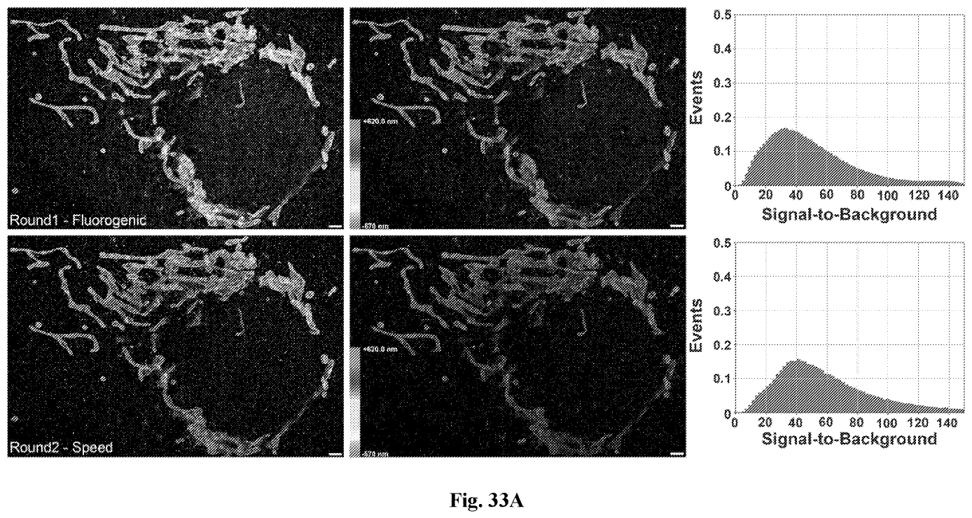

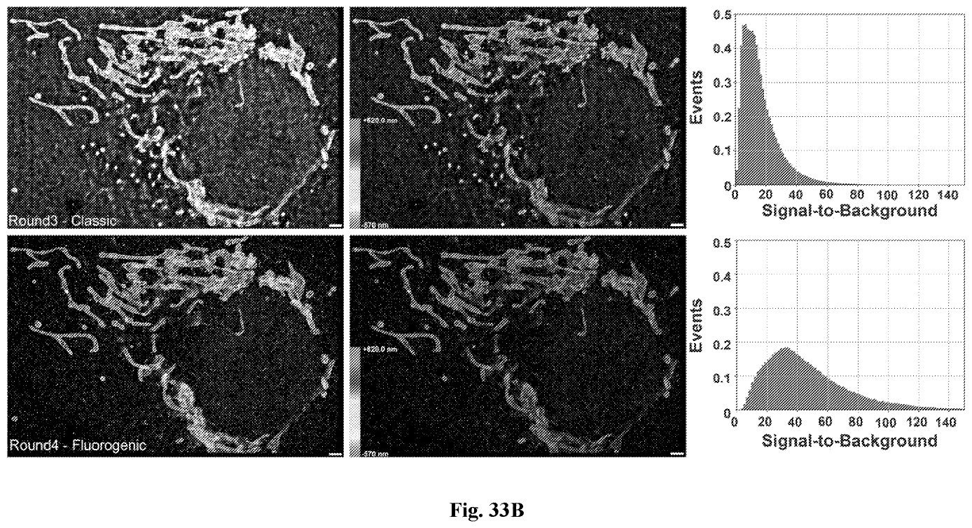

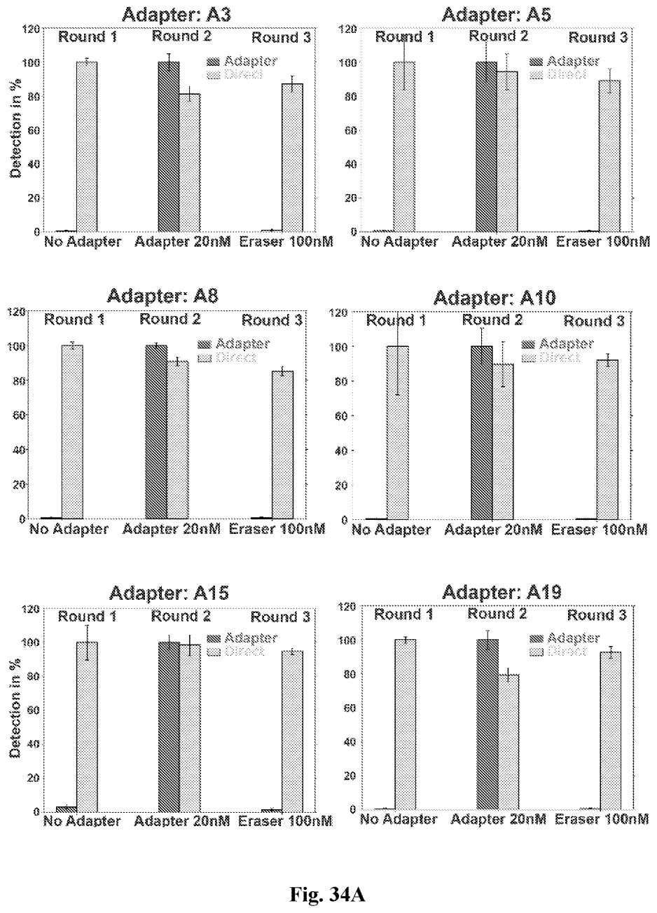

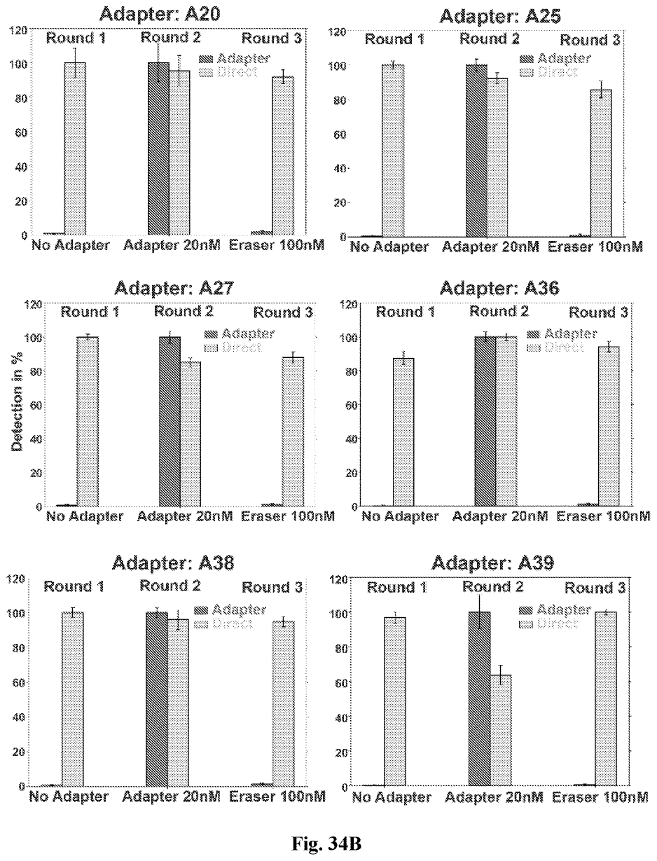

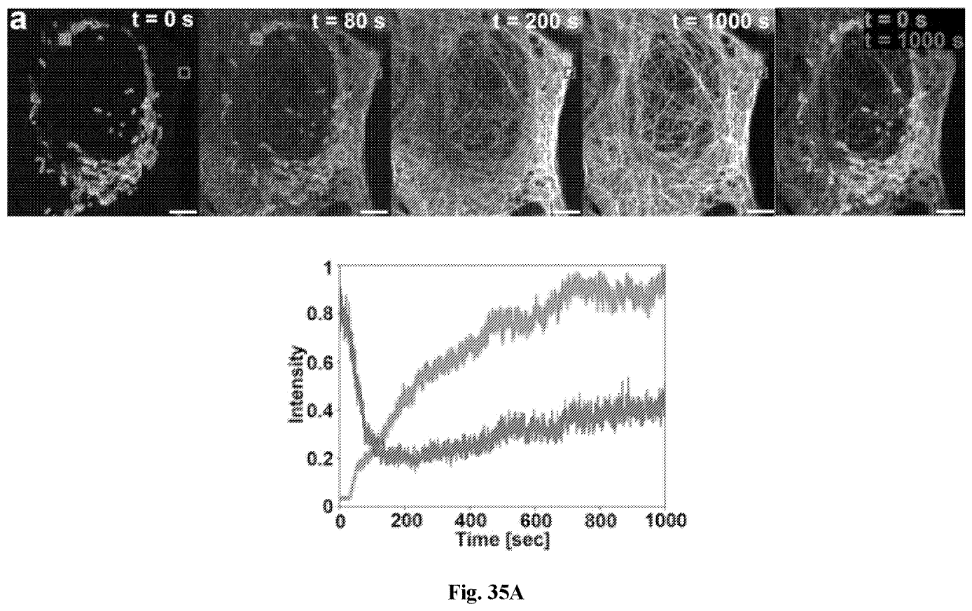

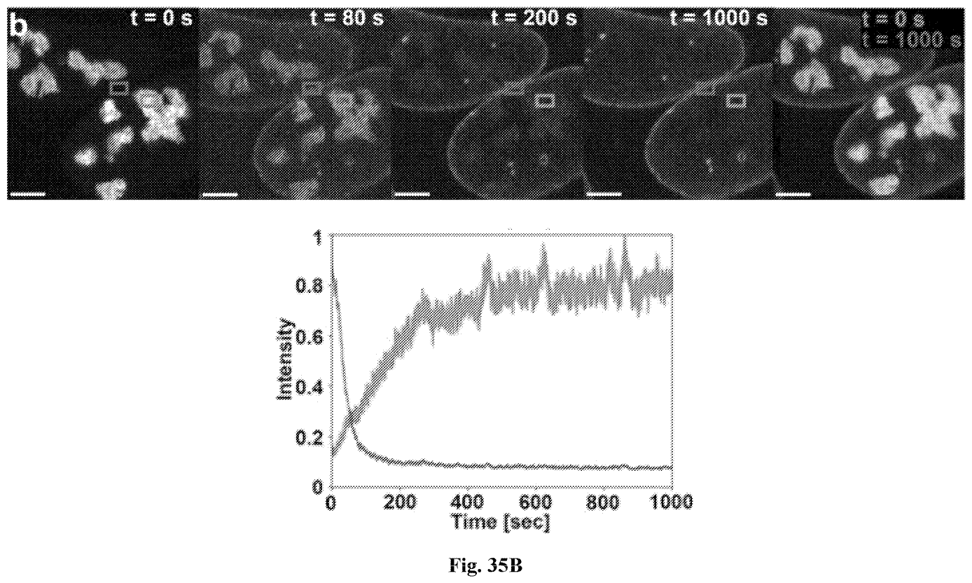

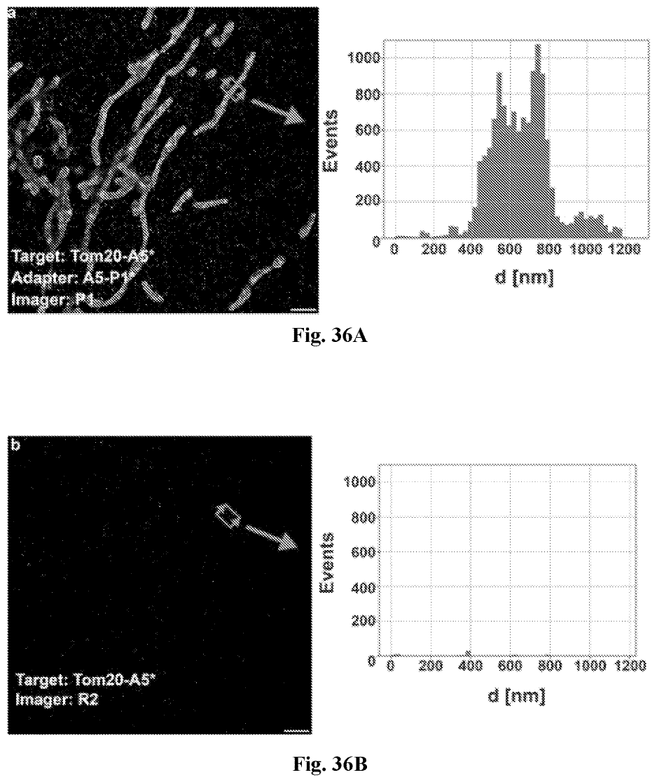

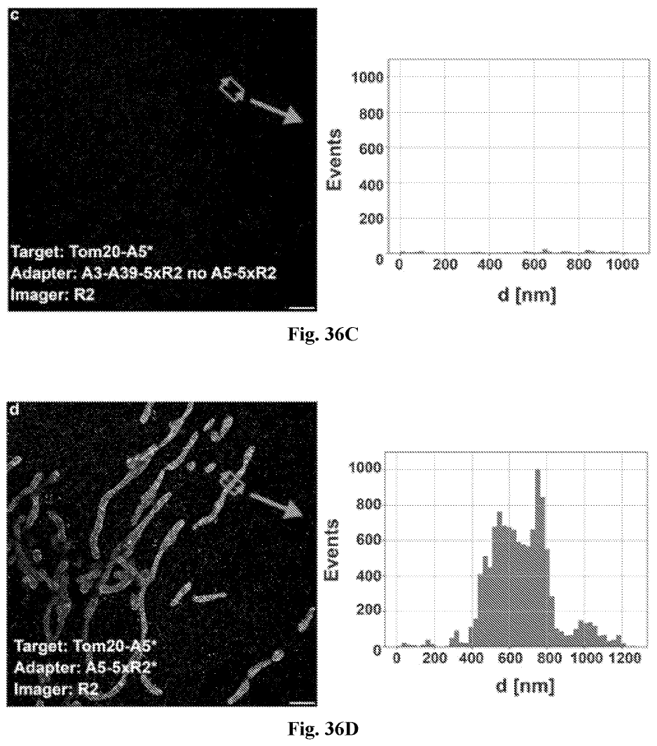

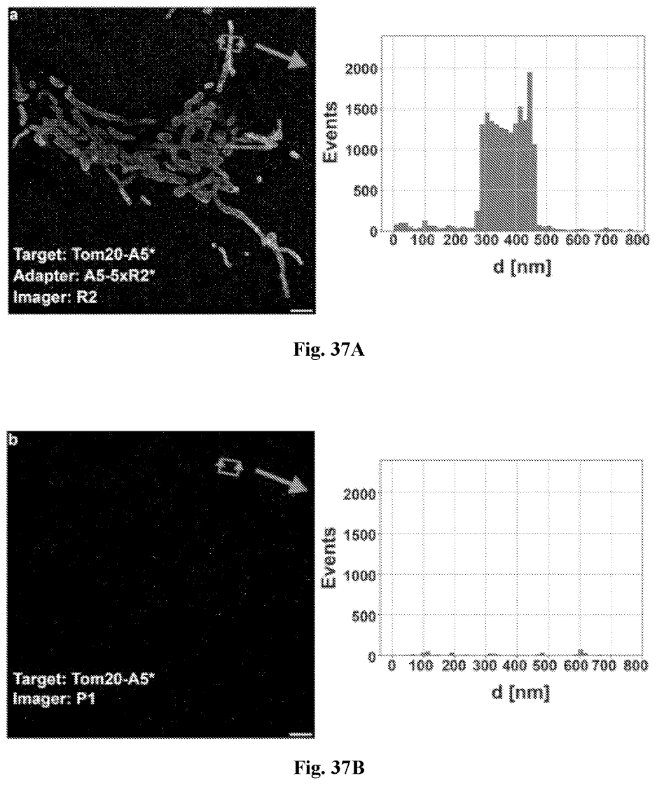

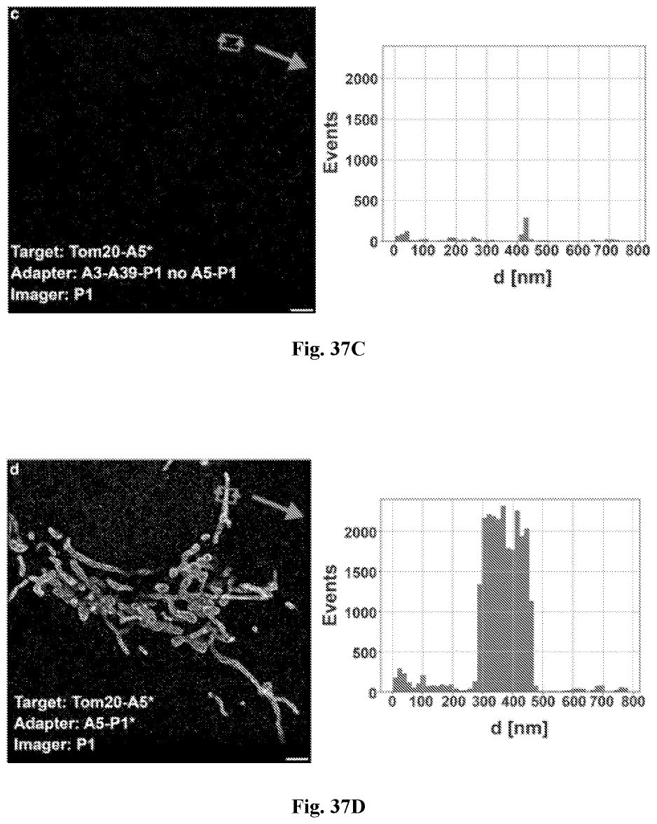

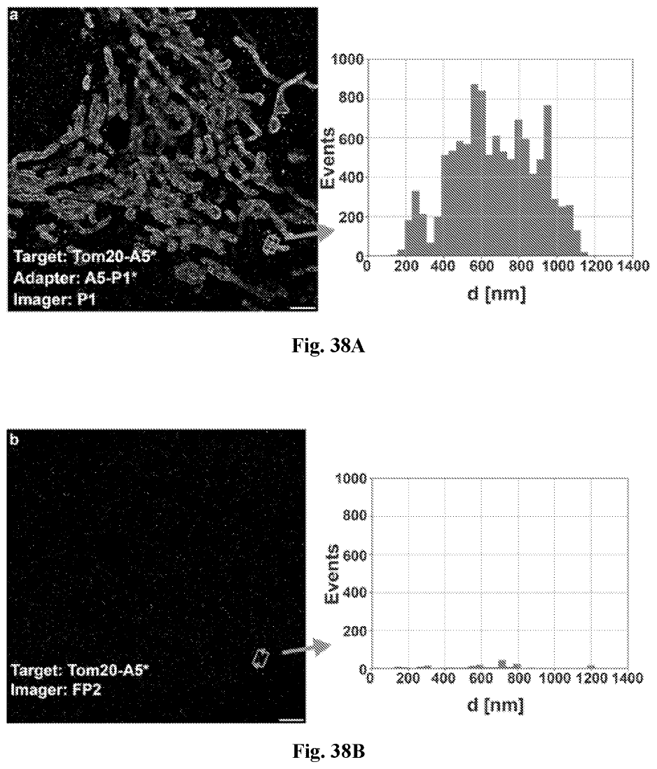

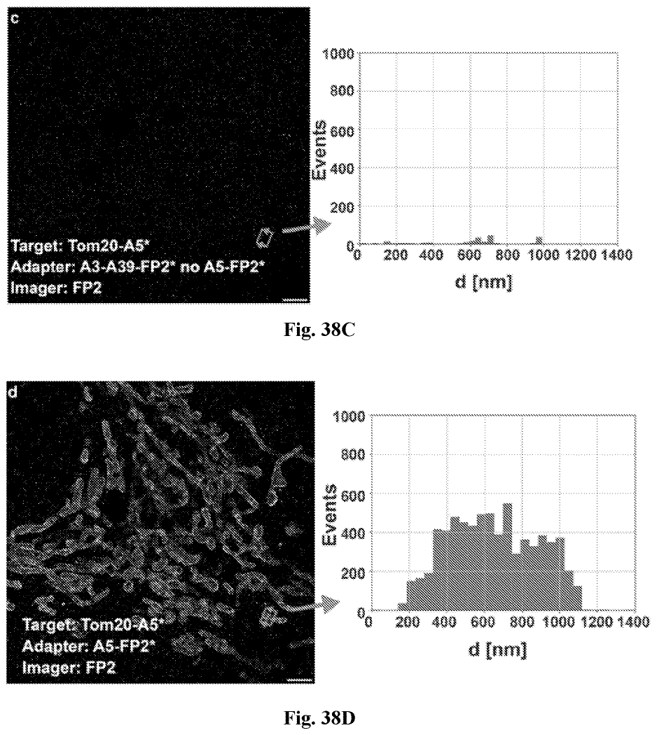

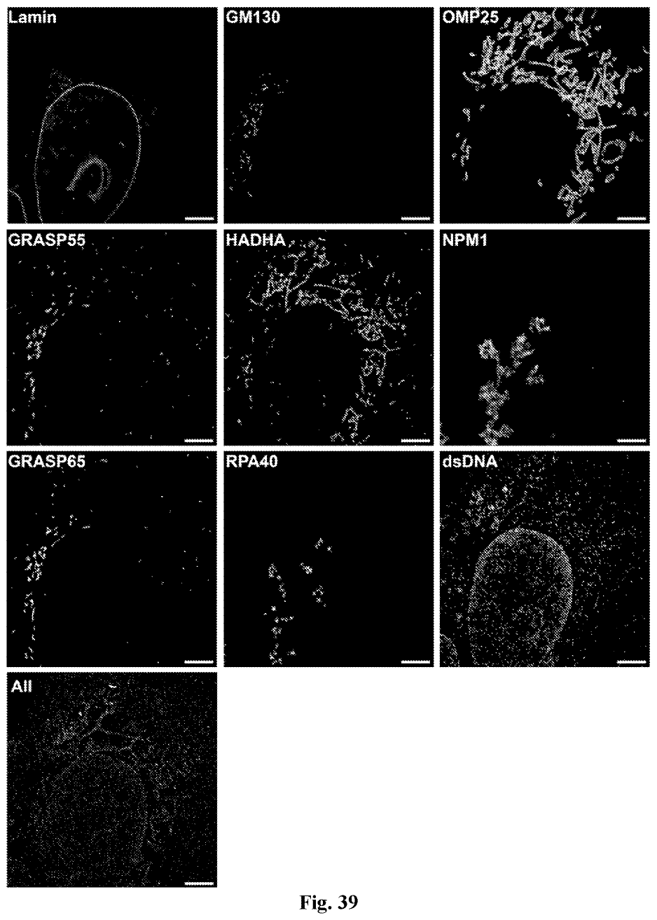

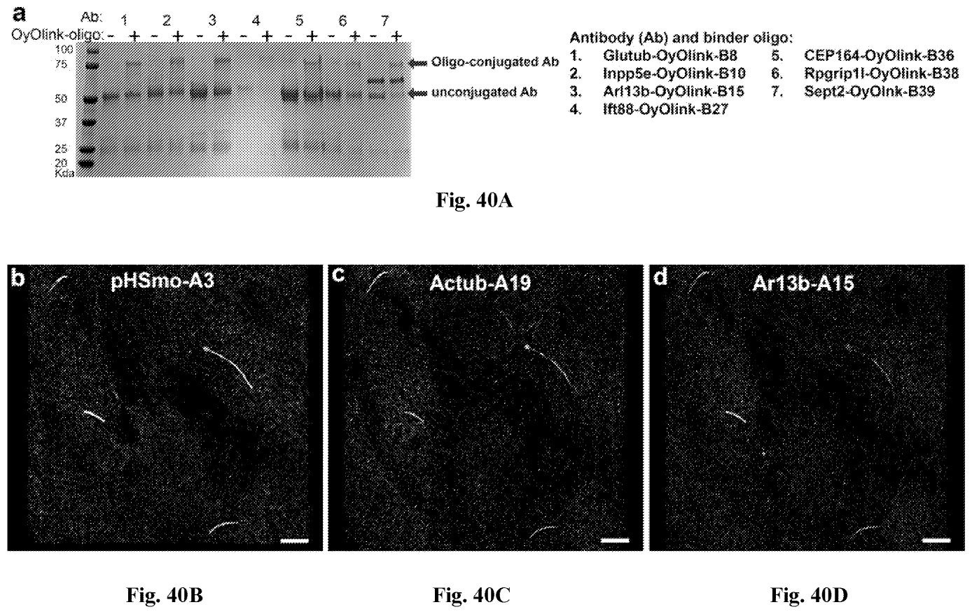

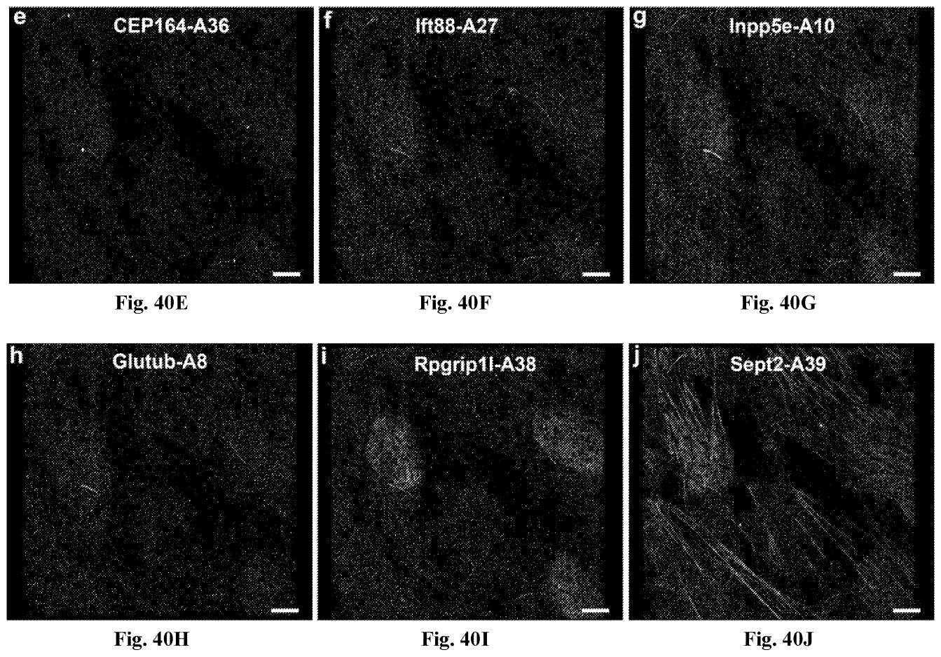

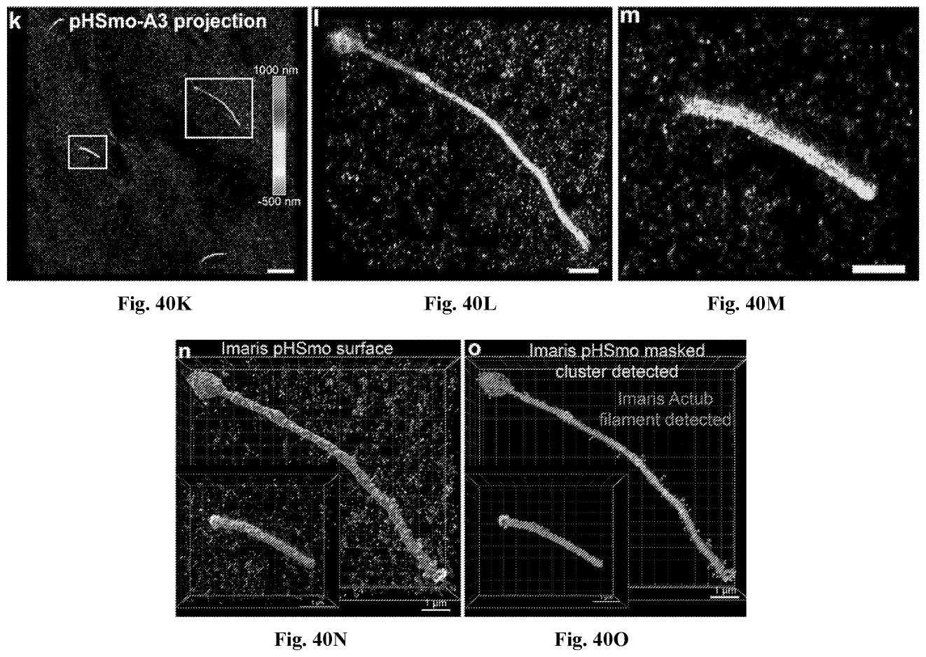

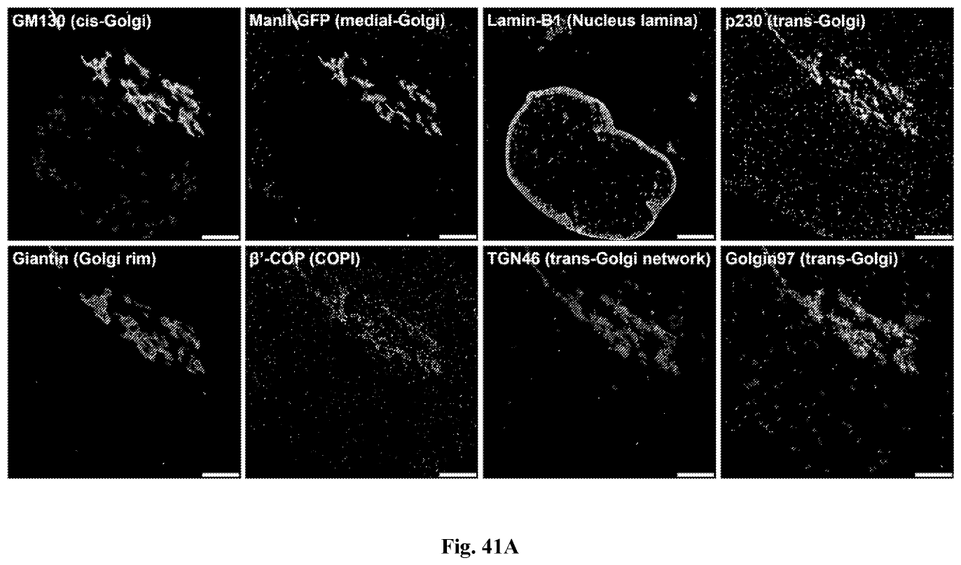

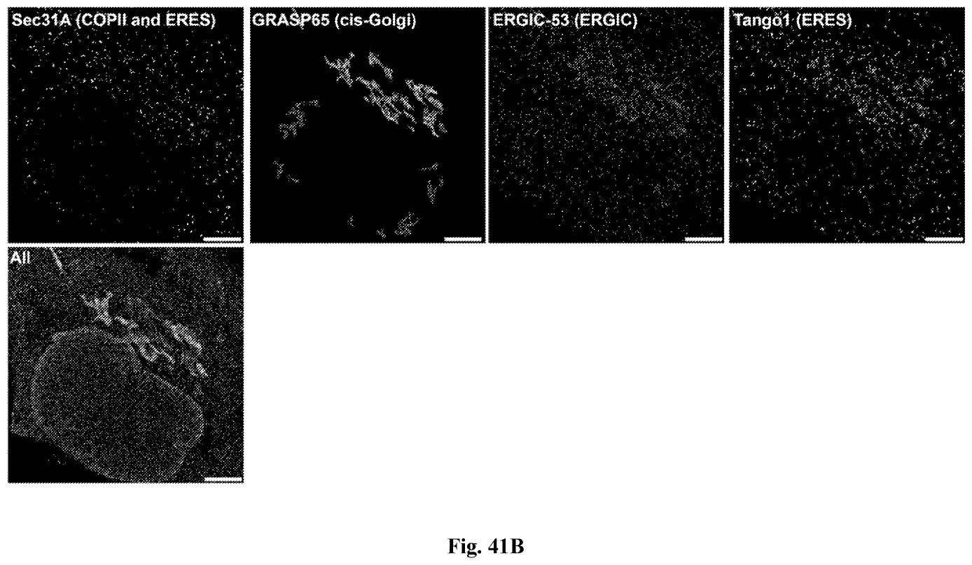

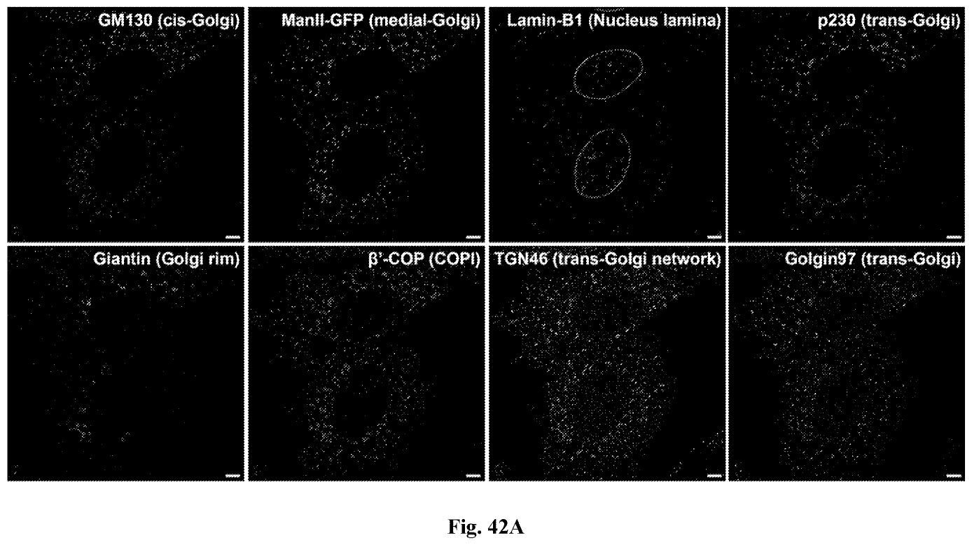

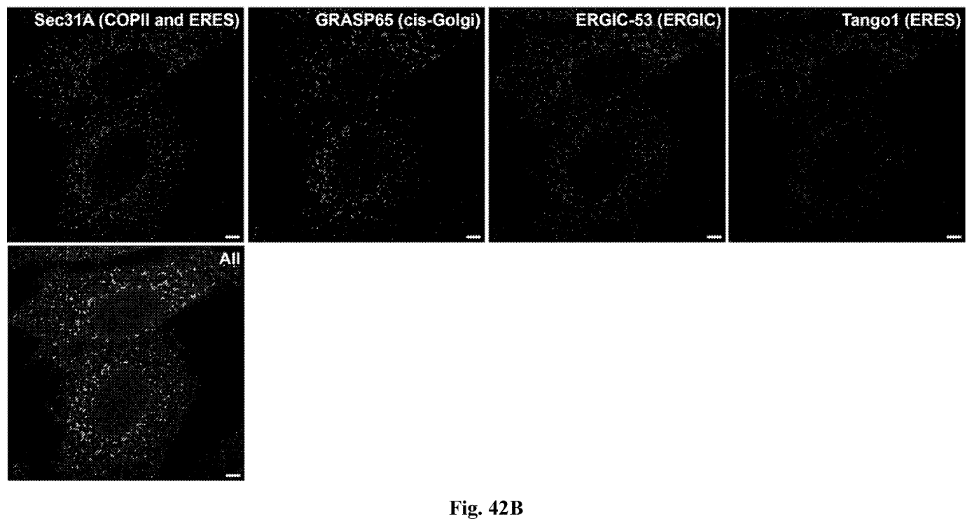

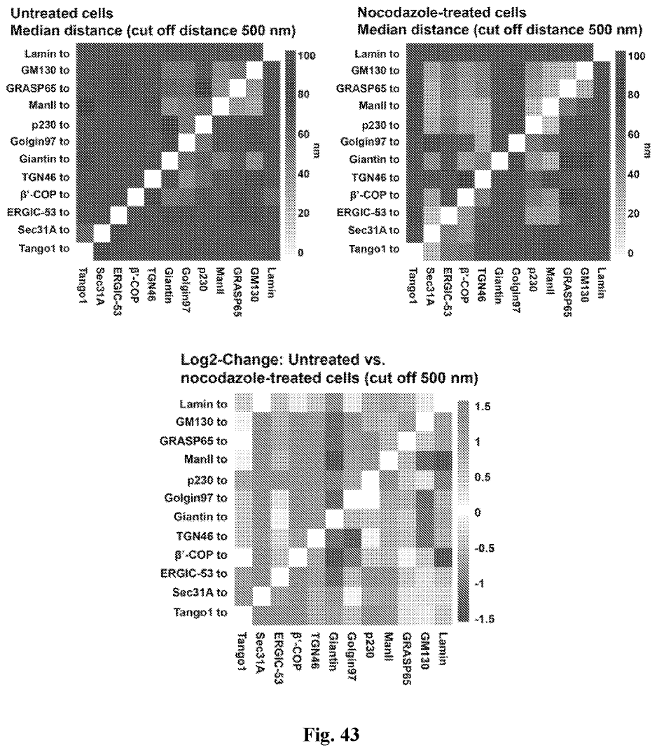

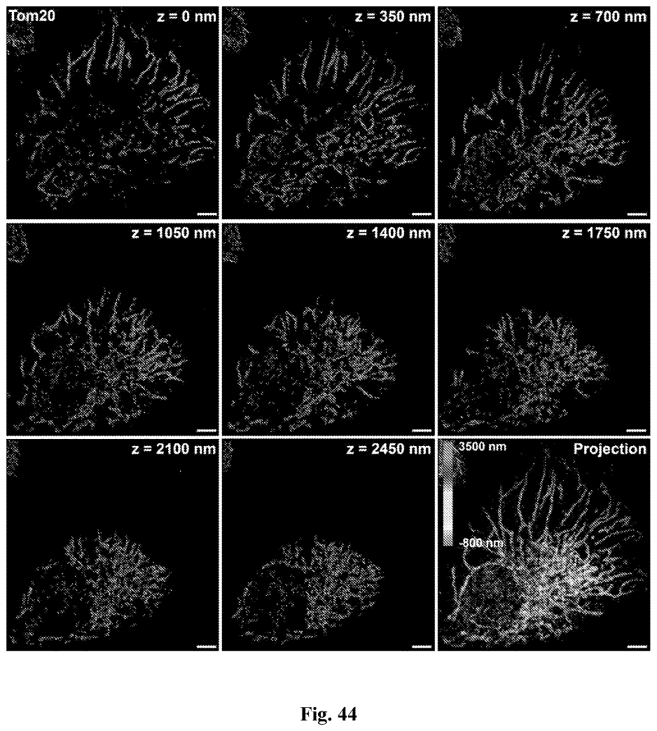

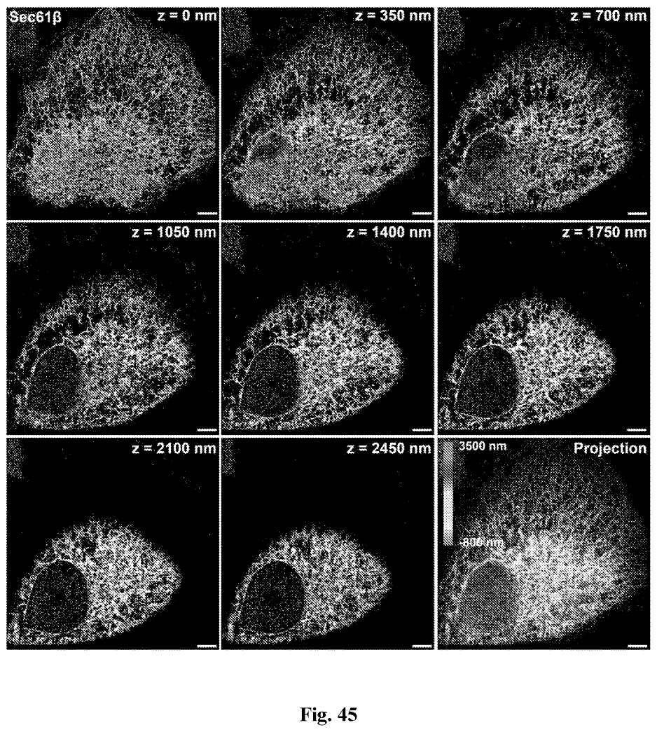

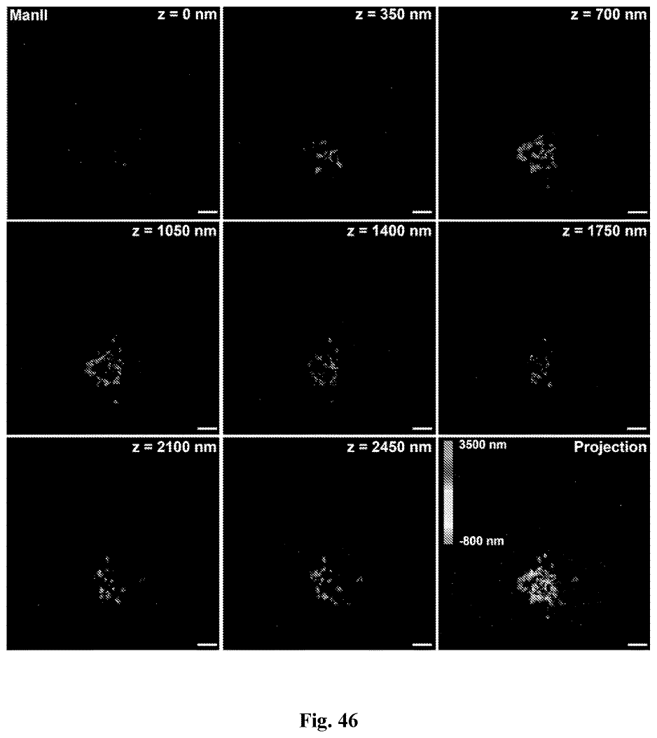

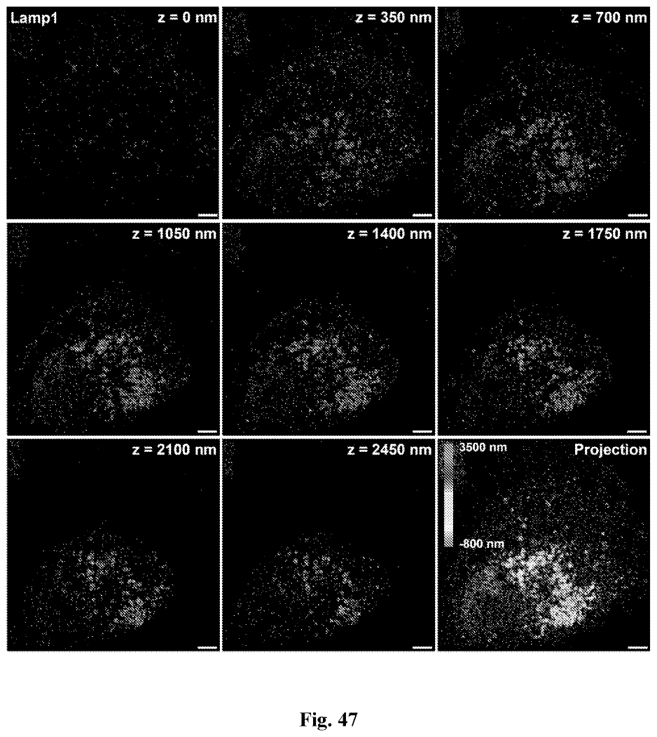

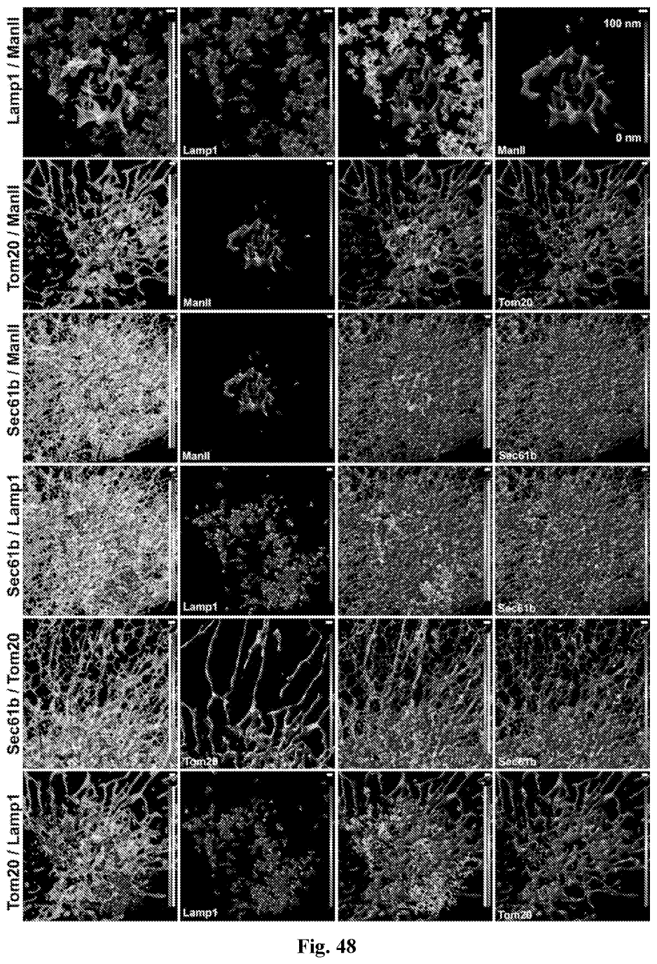

FIGS. 41A-41B: Images of the individual imaging rounds of FIGS. 17A-17G, in accordance with some embodiments. Additionally to the single-target imaging rounds, for alignment purposes, the sample is imaged an additional time with all targets simultaneously. Scale bars: 5 μm.

FIGS. 42A-42B: Images of the individual imaging rounds of FIGS. 17I-17O, in accordance with some embodiments. Additionally to the single-target imaging rounds, for alignment purposes, the sample is imaged an additional time with all targets simultaneously. Scale bar: 5 μm.

FIG. 43: Distance Heatmaps and log 2 change of proteins of the secretory pathway in untreated and nocodazole-treated HeLa cells shown in FIGS. 17A-17P, in accordance with some embodiments. The heatmap on the right represents the log 2 change of distances (Distance Untreated cells/Distance Nocodazole-treated cells).

FIG. 44: Individual optical sections of the Tom20 super-resolution image in FIG. 18B, in accordance with some embodiments. The axial spacing between the optical sections is 350 nm. The last panel shows a z-projection with the color denoting the z-position of the localization events. Scale bar: 5 μm.

FIG. 45: Individual optical sections of the Sec61ß super-resolution image in FIG. 18A, in accordance with some embodiments. The axial spacing between the optical sections is 350 nm. The last panel shows a z-projection with the color denoting the z-position of the localization events. Scale bar: 5 μm.

FIG. 46: Individual optical sections of the ManII super-resolution image in FIG. 18D, in accordance with some embodiments. The axial spacing between the optical sections is 350 nm. The last panel shows a z-projection with the color denoting the z-position of the localization events. Scale bar: 5 μm.

FIG. 47: Individual optical sections of the Lamp1 super-resolution image in FIG. 18A, in accordance with some embodiments. The axial spacing between the optical sections is 350 nm. The last panel shows a z-projection with the color denoting the z-position of the localization events. Scale bar: 5 μm.

FIG. 48: Contact sites between the ER (Sec61B), mitochondria (Tom20), lysosomes (Lamp1) and the Golgi complex (ManII-GFP), in accordance with some embodiments. The first and third columns display the pairs of organelles for which contact sites are calculated. The contact site is computed for the ‘blue’ organelle with respect to the ‘yellow’ organelle. The second and fourth columns present the corresponding contact site maps for the organelles shown in the first or third column. The colormap represents distances>100 nm in blue and distances<100 nm in white and red (see color bar). Scale bar: 1 μm.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

A “transient” adapter can be a molecule that decouples from a target of interest without the need to apply heat, a denaturing agent such as formaldehyde, or an invader strand that zips off the fluorescence reporter probe from the target of interest. In this manner, a transient adapter can bind temporarily or non-permanently. Transiency can be assessed with respect to an experiment or procedure such as an adapter can be said to be transient if a certain portion (e.g., of or approaching 100%, e.g., about 90%, 95%, 98%, 99%, and the like) will bind and unbind from the target within the course of the experiment or procedure. Transiency can also be assessed with regard to binding duration (e.g., between about 1 second and 1 minute). The binding time is usually a statistical distribution in time in which the mean value is reported as “binding time”. A transient adapter can have a washing efficiency of or approaching 100%, e.g., about 90%, 95%, 98%, 99%, and the like. A transient adapter can, but need not, be effectively removed solely by flushing (e.g., with water or buffer). A transient adapter need not be washed in order to unbind from a target (although the unbinding may also be transient before rebinding to a target).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

According to some embodiments herein, the terms “first/second target” refers to molecules that specifically bind to and label a point of interest in the sample, for example, a molecule, a complex, a structure, an organelle, a wild-type, engineered or mutated nucleic acid sequence, a peptide or protein, or a cell in a sample. The “first/second target” binds indirectly to the “first/second imaging molecule” via the “first/second adapter,” which binds to both the “first/second target” and the “first/second imaging molecule” simultaneously. The indirect interaction between the “first/second target” and the “first/second imaging molecule” allows the detection of the molecule, complex, structure, organelle, or cell in the sample, via the detection of the “first/second imaging molecule.”

In various embodiments, the first adapter and its associated imaging molecule may be disassociated from the first target, in various embodiments by contacting the sample with an eraser molecule, and a second target may be applied to label and enable the detection of the same or a different point of interest. The first/second distinction refers to this removal and addition of targets and accordingly there may be multiple first targets applied simultaneously or sequentially prior to the application of an eraser or other means of removing the first target or targets.

In some embodiments, the first target and the second target (and additional target(s), if any) are applied at the same time. According to these embodiments, after the application of the eraser to disrupt the first target-first adapter-first imaging molecule association, there is no need to apply the second target; rather, the second adapter and the second imaging molecule can be applied directly on the sample since the second target is already in the same and labels the point-of-interest that the second target has specificity for.

### DETAILED DESCRIPTION

DNA-PAINT has emerged as one of the most promising super-resolution microscopy methods in the last couple of years. The most prominent advantages are: Spectrally unlimited multiplexing (Exchange-PAINT), high spatial resolution (sub-5 nm), and the capability of counting target of interest molecules via the very predictable binding kinetics. However, an undisputed problem was the comparably slow imaging speed because the apparent blinking of DNA-PAINT is based on diffusion of dye-labeled DNA oligos (“Imagers”) and transient binding of these to the target of interest.

In the last two years, rational sequence design and fluorogenic imagers have addressed this problem, achieving up to two orders of magnitude faster DNA-PAINT. However, the problem with these approaches is that the sequence design of these Imagers is drastically reducing the sequence space, which ultimately limits the multiplexing capability (>6 targets of interest).

In other words, traditional DNA-PAINT has been either very slow and susceptible for background or very limited in multiplexing space. This stands in a strong contrast to the needs of a spatial omics experiment.

To make DNA-PAINT the go-to tool for spatial omics experiments and in vitro binding assays, it is essential to enable fastest and highly multiplexed DNA-PAINT.

Applicant designed ‘adapters’ for DNA-PAINT that will enable fastest and highly multiplexed Fluorogenic-PAINT to address this issue.

Although certain embodiments of the invention may be described in the context of DNA-PAINT, the invention has broad applicability and provides advantages in confocal imaging, expansion microscopy, and other dye-based fluorescence imaging modalities.

In this concept, the binding of the Imager strand is mediated via an adapter strand, which transiently binds to the docking site at the target of interest. The adapter strand, therefore, has at least two regions.

The first part is the binding site for the Imager, which can be any sequence, including the already established speed sequences and the fluorogenic Imagers. This part could be the same in every round of a multiplexing experiment (i.e., for every round, the same Imager can be used).

The second part of the adapter binds to the docking site at the target of interest. This sequence can be any sequence in the transient binding regime (˜1 million sequences available). Since the adapter strand is not fluorescent and, hence, does not contribute to the background, it can be used in high concentrations (i.e., enabling a fast-binding frequency). Therefore, this part of the adapter does not need to be a speed-optimized sequence. The trick, in this case, is to enable fast imaging via a high concentration of the adapter strand and, therefore, not limit the pool of sequences for multiplexing.

Referring now to FIG. 1, left panel, a transient adapter 102 can include a first end 104 having a target-complementary sequence; and a second end 106 having a fluorescent-imaging-molecule-complementary sequence. The first end 104 and the second 106 can be directly adjacent to each other or can be separated by additional nucleotides.

FIG. 1, right panel is a timeline depicting the sequential binding of the adapter 102 and the imager 108. Ideally, the time when a given adapter docking site 110 on a target of interest (T1) is unoccupied (represented as y=0) is minimized. The binding time of an adapter can be configured using the number of nucleotides as illustrated in Table 1 below.

TABLE 1
Estimated Relationship Between Nucleotide
Length and Binding Time

| Nucleotide Length (nt) | Estimated Binding Time | |
|---|---|---|
| 8 | ~100 | ms |
| 9 | ~1 | s |
| 10 | ~10 | s |
| 11 | ~100 | s |

Table 1 represents a general relationship and binding time may be affected by one or more factors such as salinity, temperature, pH, and the like.

Because the adapter is not fluorescent, the quantity and/or concentration can be increased to decrease the time in between binding events without raising the risk of photobleaching or increasing the fluorescent background.

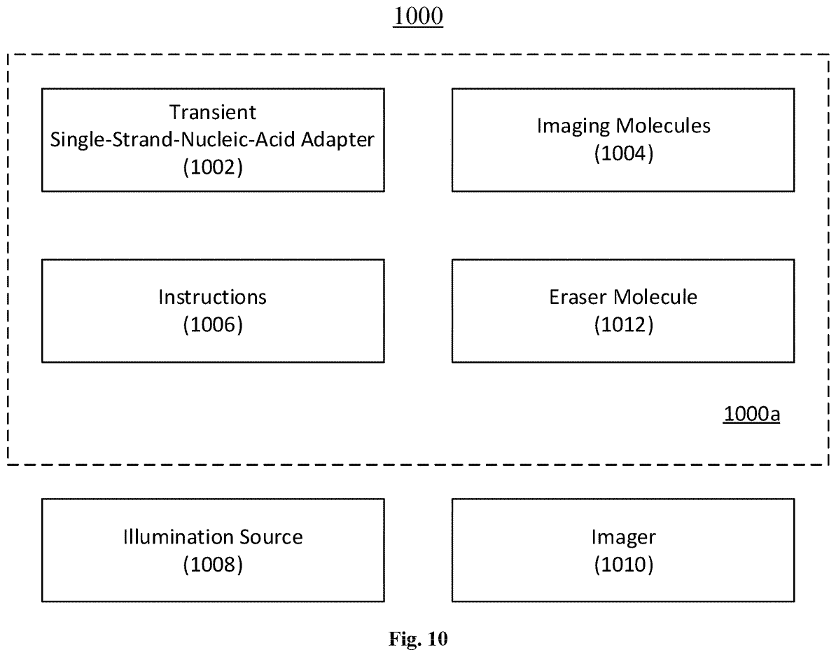

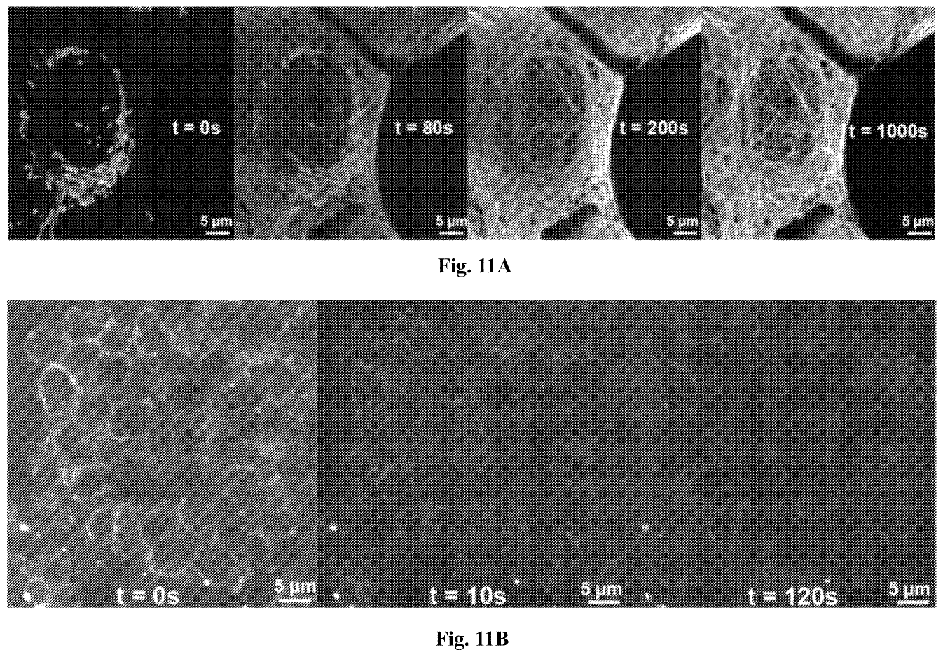

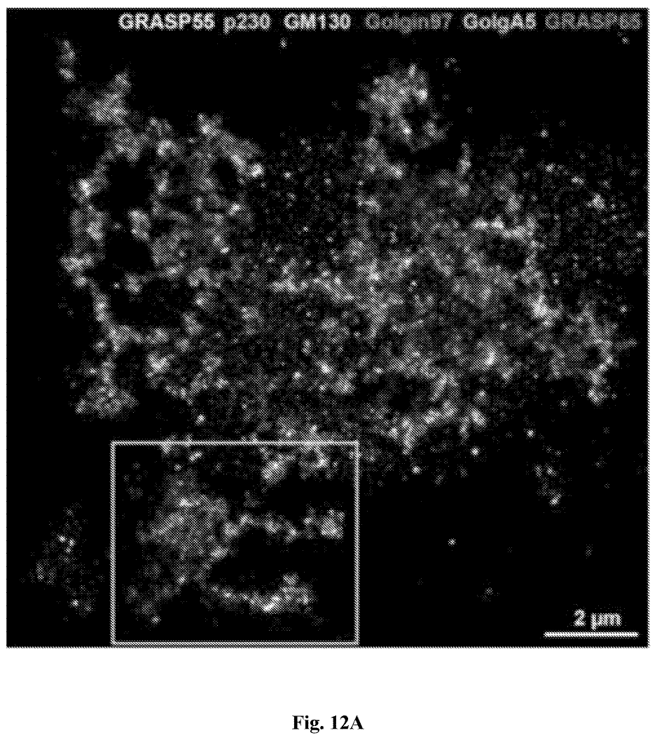

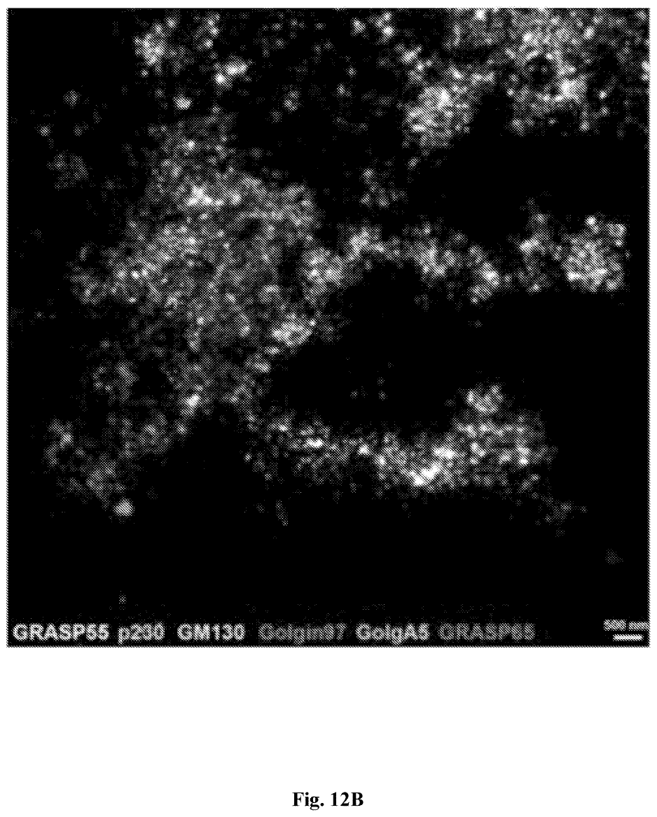

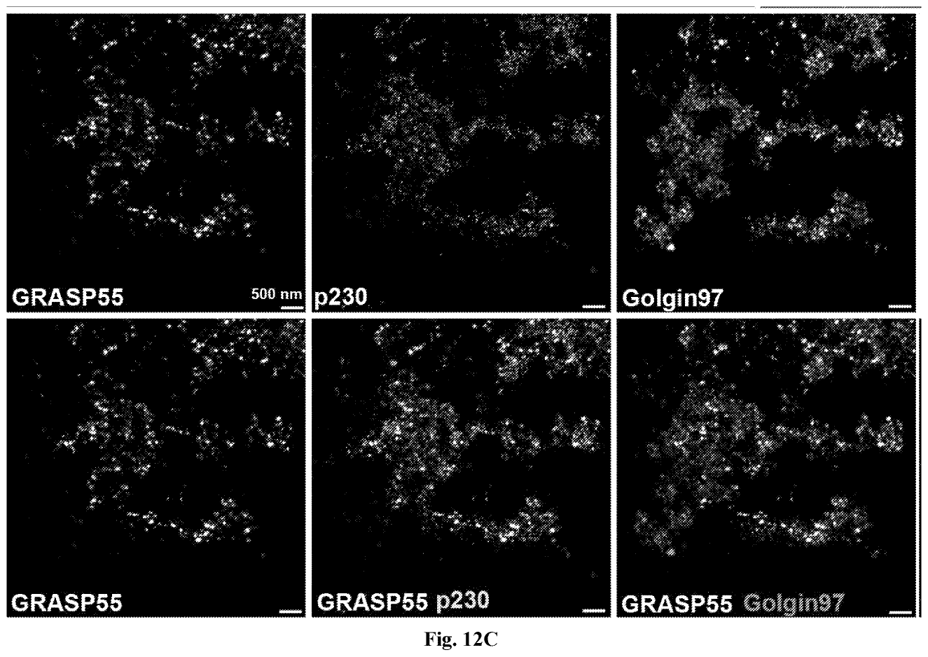

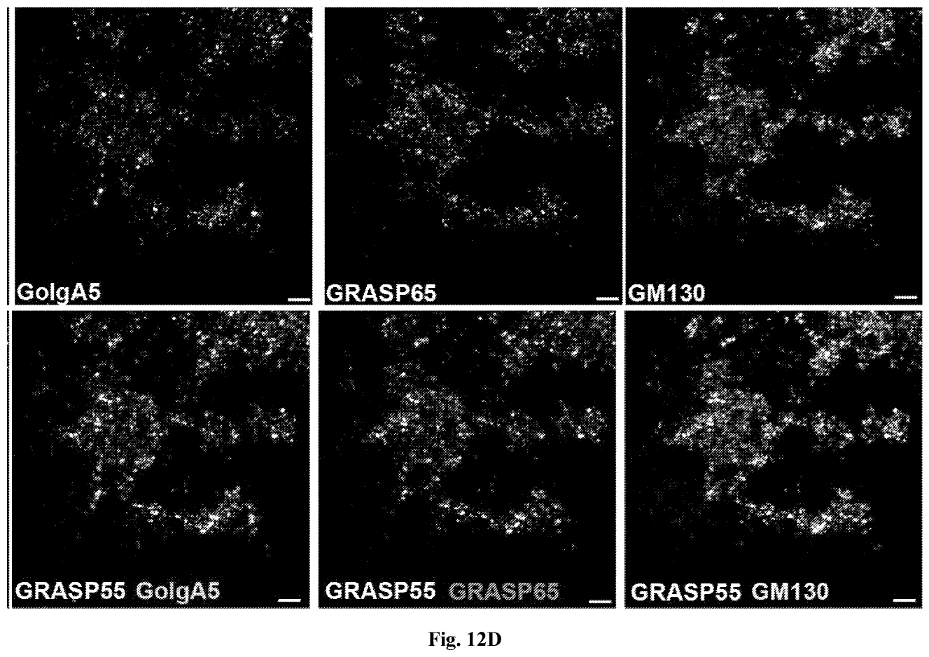

Referring to FIGS. 9 and 10, other aspects of the invention provide a method 900, system 1000, and kit 1000a for single-molecule imaging according to an embodiment of the invention. In step S902, a sample having a plurality of targets is exposed to a plurality of transient non-fluorescent single-strand-nucleic-acid adapter molecules 1002. In step S904, the sample is exposed to a plurality of fluorescent imaging molecules 1004. In step S906, the sample is exposed to an excitation source 1008 having a wavelength capable of exciting the plurality of plurality of fluorescent imaging molecules.

In step S908, the sample is exposed to an eraser molecule 1012 adapted and configured to quench the transient non-fluorescent single-strand-nucleic-acid adapter molecules. In step S902, the sample can be exposed to a second plurality of transient non-fluorescent single-strand-nucleic-acid adapter molecules having a second, different target-complementary sequence. The sample can then be exposed to a plurality of fluorescent imaging molecules. These plurality of fluorescent imaging molecules can be introduced anew or can be remain in the environment of the sample from step S904. The sample-adapter-imager complex can then be imaged again.

Imaging can be performed using a variety of imagers 1010 such as microscopes. The excitation source 1008 can be integrated within the imager 1010. The kit 1000a can include instructions, e.g., printed material detailing the methods (e.g., 900) described herein.

### Imaging Method

In some aspects, the present invention is directed to an imaging method.

In some embodiments, the method includes performing a first labeling; and acquiring a first image.

In some embodiments, performing the first labeling includes applying to a sample one of more targets comprising a first target, which comprises a first target single-strand-nucleic-acid; applying to the sample a first adapter comprising a first adapter single-strand-nucleic-acid; and applying to the sample a first imaging molecule comprising a first imaging molecule single-strand-nucleic-acid and a first detection motif.

In some embodiments, acquiring a first image includes acquiring a first image of the first detection motif.

In some embodiments, the first adapter single-strand-nucleic-acid includes: a first region having a sufficient sequence complementarity to bind the first target single-strand-nucleic-acid; and a second region having a sufficient sequence complementarity to bind the first imaging molecule single-strand-nucleic-acid.

In some embodiments, the first adapter mediates an association between the first target and the first imaging molecule.

In some embodiments, the first target is used to label a molecule, a complex, a structure, an organelle, and etc, in the sample such that the first imaging molecule can be located to the molecule, complex, structure, or organelle via the first target and the first adapter. This way, the molecule, complex, structure, or organelle can be detected by the imaging method herein through the detection of the first detection motif.

The nature of the first target is not limited. One of ordinary skill in the art would understand that various molecules/structures in a sample can be labeled in various ways. For example, the first target can include an antibody, a non-antibody protein that specifically interact with a molecule or a structure, a nucleic acid, and the like. Antibodies or polypeptides that specifically bind to a protein, a protein complex, a nucleic acid, a cell structure, a cell organelle, or a cell a widely available in the art and can be readily selected by one of ordinary skill in the art. Similarly, nucleic acids that specifically bind to or is complementary with a nucleic acid of interest are also widely available based on the specific imaging experiment.

In some embodiments, the first target, the first adapter, the first imaging molecule, etc., are not merely one first target, one first adapter or one first imaging molecule. One of ordinary skill in the art, reading in light of the instant specification, would understand that the imaging method herein allows the use of multiple first targets, first adapters, first imaging molecules to labeled and image multiple molecules, complexes, structures, organelles, etc., in the sample at the same time.

One of ordinary skill in the art, reading in light of the instant specification, would understand that the choice of the first detection motif is not limited. Detection motifs can be chosen based on the available imaging device.

For example, if the imaging device is a fluorescence microscopy, the first detection motif can be a fluorescence motif, such as a fluorescent protein (GFP, RFP, YFP, CFP, etc), a fluorescent small molecule (a xanthene derivative, a cyanine derivative, a squaraine derivative or a ring-substituted squaraine, a squaraine rotaxane derivative, a naphthalene derivative, a coumarin derivative, an oxadiazole derivative, an anthracene derivative, a pyrene derivative, an oxazine derivative, an acridine derivative, an arylmethine derivative, a tetrapyrrole derivative, a dipyrromethene derivative, and etc.), a quantum dot, and the like.

For example, if the imaging device is an optical microscope or other types of microscopes that can detect metal nanoparticles, the first detection motif can be a metal nanoparticle, such as a gold nanoparticle, a tungsten nanoparticle, a silica nanoparticle, an iron nanoparticle, a copper nanoparticle, a selenium nanoparticle, a molybdenum nanoparticle, a silver nanoparticle, a gadolinium nanoparticle, a holmium nanoparticle, a rhenium nanoparticle, a platinum nanoparticle, and the like.

For example, if the imaging device is suitable for performing Raman scattering microscopy or otherwise detect Raman scattering, the first detection motif can be Raman scattering motif, such as a Raman dye, such as a Raman dye suitable for a stimulated Raman scattering microscopy.

For example, if the imaging device is able to detect radioactivity or isotopes of elements, the first detection motif can be an isotope.

In some embodiments, the number of complementary base pairs, Kₒₙ, K_{off}, K_{d} between the first target single-strand-nucleic-acid and the first region of the first adapter, between the first imaging molecule single-strand-nucleic-acid and the second region of the adapter, and/or between the first imaging molecule single-strand-nucleic-acid and the second region of the adapter is chosen based on the specific experiment, such that the indirect association between the first target and the first imaging molecule is suitable for the imaging device to specifically detect the first imaging molecule anchored to the molecule, complex, structure, organelle or cell the first target labels, and that this indirect association can be easily disrupted/outcompeted by the eraser molecule (described elsewhere herein) to allow another round of labeling and detection.

In some embodiments, a number of complementary base pairs between the first target single-strand-nucleic-acid and the first region of the first adapter ranges between 1-30, such as between 5-20, or between 8-12. In some embodiments, number of complementary base pairs between the first target single-strand-nucleic-acid and the first region of the first adapter ranges is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or any ranges therebetween.

In some embodiments, a Kₒₙ between the first target single-strand-nucleic-acid and the first region of the adapter ranges between about 1*10³ l/M*s and about 1*10⁸ l/M*s, such as between about 1*10⁴ l/M*s and about 1*10⁷ l/M*s, or between about 1*10⁵ l/M*s and about 1*10⁶ l/M*s. In some embodiments, the Kₒₙ between the first target single-strand-nucleic-acid and the first region of the adapter is about 1*10³ l/M*s, about 1*10⁴ l/M*s, 1*10⁵ l/M*s, 1*10⁶ l/M*s, 1*10⁷ l/M*s, 1*10⁸ l/M*s, or any ranges therebetween.

In some embodiments, a K_{off} between the first target single-strand-nucleic-acid and the first region of the adapter ranges between about 10 l/s and about 0.00001 l/s, such as between about 1 l/s and about 0.0001 l/s, between about 0.1 l/s and about 0.001 l/s, or between about 0.03 l/s and about 0.003 l/s. In some embodiments, a K_{off} between the first target single-strand-nucleic-acid and the first region of the adapter is about 10 l/s, such as about 1 l/s, 0.1 l/s, 0.01 l/s, 0.001 l/s, 0.0001 l/s, 0.00001 l/s, or any ranges there between.

In some embodiments, a K_{d} between the first target single-strand-nucleic-acid and the first region of the adapter ranges between 100 μM and 0.1 nM, such as between 10 μM and 1 nM, between 1 μM and 10 nM, or between 300 nM and 30 nM. In some embodiments, the K_{d} between the first target single-strand-nucleic-acid and the first region of the adapter is about 100 μM, about 10 μM, about 1 μM, about 100 nM, about 10 nM, about 1 nM, or any ranges therebetween.

In some embodiments, a number of complementary base pairs between the first imaging molecule single-strand-nucleic-acid and the second region of the adapter ranges between 1-30, such as between 5-20, or between 8-12. In some embodiments, the number of complementary base pairs between the first imaging molecule single-strand-nucleic-acid and the second region of the adapter ranges is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or any ranges therebetween.

In some embodiments, a Kₒₙ between the first imaging molecule single-strand-nucleic-acid and the second region of the adapter ranges between about 1*10³ l/M*s and about 1*10⁸ l/M*s, such as between about 1*10⁴ l/M*s and about 1*10⁷ l/M*s, or between about 1*10⁵ l/M*s and about 1*10⁶ l/M*s. In some embodiments, the Kₒₙ between the first imaging molecule single-strand-nucleic-acid and the second region of the adapter is about 1*10³ l/M*s, about 1*10⁴ l/M*s, 1*10⁵ l/M*s, 1*10⁶ l/M*s, 1*10⁷ l/M*s, 1*10⁸ l/M*s, or any ranges therebetween.

In some embodiments, a K_{off} between the first imaging molecule single-strand-nucleic-acid and the second region of the adapter ranges between about 10000 l/s and about 0.00001 l/s, such as between about 1000 l/s and about 0.0001 l/s, between about 100 l/s and about 0.001 l/s, between about 10 l/s and about 0.01 l/s, between about 1 l/s and about 0.1 l/s. In some embodiments, a K_{off} between the first imaging molecule single-strand-nucleic-acid and the second region of the adapter is about 10000 l/s, about 1000 l/s, about 100 l/s, about 10 l/s, about 1 l/s, 0.1 l/s, 0.01 l/s, 0.001 l/s, 0.0001 l/s, 0.00001 l/s, or any ranges there between.

In some embodiments, a K_{d} between the first imaging molecule single-strand-nucleic-acid and the second region of the adapter ranges between 100 μM and 0.1 nM, such as between 10 μM and 1 nM, between 1 μM and 10 nM, or between 300 nM and 30 nM. In some embodiments, the K_{d} between the first imaging molecule single-strand-nucleic-acid and the second region of the adapter is about 100 μM, about 10 μM, about 1 μM, about 100 nM, about 10 nM, about 1 nM, or any ranges therebetween.

As described elsewhere herein, the first target, first adapter, first imaging molecule, etc., can be more than one during the first labeling such that more than one point of interest (e.g., molecule, complex, structure, organelle, cell, etc.) in the sample can be labeled and detected at the same time without removing the labeling first.

Accordingly, in some embodiments, performing the first labeling includes: applying to the sample a plurality of first targets, each comprising a first target single-strand-nucleic-acid; applying to the sample a plurality of first adapters, each comprising a first adapter single-strand-nucleic-acid; and applying to the sample a plurality of first imaging molecules, each comprising a first imaging molecule single-strand-nucleic-acid and a first detection motif. In some embodiments, acquiring the first image includes acquiring images of the plurality of first detection motifs of the plurality of first imaging molecules. In some embodiments, each of the first adapters mediates an association of each of the plurality of first targets and each of the plurality of the first imaging molecules in a sequence-specific manner. In some embodiments, the plurality of first detection motifs do not interfere with each other during the acquisition of the first image.

In some embodiments, a detection motif interferes with another detection motif if the detection signals given by the two motifs overlap or are the same. For example, the emission spectrum of a molecule of enhanced green fluorescent protein (EGFP) is the same as another molecule of EGFP, and thus the fluorescence signals from the two molecules interfere with each other. For another example, the emission spectrum of a molecule of EGFP overlaps with the emission spectrum of a molecule of enhanced cyan fluorescent protein (ECFP), and therefore the two molecules may interfere with each other for some fluorescence microscopes.

One feature of the imaging method herein is that the method allows the first imaging molecule to be dissociated from the first target without the washing or stripping the sample (which often causes disruption in the sample and/or reduction in the quality of the images acquired post-washing/stripping). Rather, according to the imaging method herein, the first imaging molecule can be easily dissociated from the first target by a nucleic acid that competes with one or more of the hybridizations required for forming the first target-first adapter-first imaging molecule complex. It is worth noting that the method herein can include washing/stripping; these steps are not essential, not incompatible.

Accordingly, in some embodiments, the imaging method further includes applying to the sample an eraser molecule to disrupt the association between the first target and the first imaging molecule mediated by the first adapter; performing a second labeling; and acquiring a second image.

In some embodiments, performing the second labeling includes: applying to a sample a second target comprising a second target single-strand-nucleic-acid; applying to the sample a second adapter comprising a second adapter single-strand-nucleic-acid; and applying to the sample a second imaging molecule comprising a second imaging molecule single-strand-nucleic-acid and a second detection motif.

In some embodiments, performing the second labeling does not include applying to a sample a second target. According some embodiments, in the first labeling, the one or more targets applied to the sample already includes both the first target and the second target. According to these embodiments, only one application of target to the sample is needed.

In some embodiments, acquiring the second image includes acquiring a second image of the second detection motif.

In some embodiments, the description of the first target, the first adapter and/or the first imaging molecule also apply to the second target, the second adapter and/or the second imaging molecule.

In some embodiments, the first detection motif and the second detection motif interfere with each other, such as producing overlapping or the same signals.

In some embodiments, the second adapter single-strand-nucleic-acid includes a third region having a sufficient complementarity to bind the second target single-strand-nucleic-acid; and a fourth region having a sufficient complementarity to bind the second imaging molecule single-strand-nucleic-acid. In some embodiments, the second adapter mediates an association between the second target and the second imaging molecule.

In some embodiments, the eraser molecule includes an eraser molecule single-strand-nucleic-acid having a sufficient sequence complementarity to bind the first region or the second region of the first adapter. In some embodiments, the eraser molecule prevents the hybridization between the first target single-strand-nucleic-acid and the first region of the adapter. In some embodiments, the eraser molecule prevents the hybridization between the first imaging molecule single-strand-nucleic-acid and the second region of the adapter.

In some embodiments, the first target, the first adapter, the first imaging molecule and the eraser molecule are not washed away from the sample before the application of the second target, the second adapter, and the second imaging molecule.

Ins some embodiments, a signal the first detection motif and a signal of the second detection motif overlap or are the same.

In some embodiments, in either or each of the first labeling and the second labeling, 2 or more, such as 3 or more, 4 or more, 5 or more, 6 or more or 7 or more of different detection motifs having different signals are used. In some embodiments, in either or each of the first labeling and the second labeling, about 2, about 3, about 4, about 5, about 6, about 7, about 8 about 9, or about 10 different detection motifs having different signals are used.

In some embodiments, the sample is expanded according to an expansion microscopy technology. Expansion microscopy technology is described in, for example, M′Saad et al., (Nature Communications volume 11, Article number: 3850 (2020)).

### Device

In some aspects, the present invention is directed to a device, such as a device for acquiring imaging, such as a device for performing the imaging method herein.

In some embodiments, the device includes: a sample holder for holding a sample; a computer-operated liquid applicator for applying a liquid to the sample; a computer-operated microscope; and a computer.

In some embodiments, the computer is programmed to: (a) operate the liquid applicator to perform a first application of: one or more targets comprising a first target for specifically binding to a first component in the sample; a first imaging molecule comprising a first detection motif detectable by the microscope; and a first adapter for mediating an association between the first target and the first imaging molecule.

In some embodiments, the computer is programmed to: (b) operate the microscope to record a first signal of the first detection motif.

In some embodiments, the computer is programmed to: (c) operate the liquid applicator to perform a second application of: an eraser molecule for interrupting the first adapter-mediated interaction between the first target and the first adapter; a second target for specifically binding to a second component in the sample; a second imaging molecule comprising a second detection motif detectable by the microscope; and a second adapter for mediating an association between the second target and the second imaging molecule. In some embodiments, the second target is among the one or more targets applied in (a) (together with the first target), and (c) thus does not include the application of the second target.

In some embodiments, the computer is programmed to: (d) operate the microscope to record a second signal of the second detection motif,

In some embodiments, the computer is programmed to perform operations (a), (b), (c) and (d) sequentially in this order.

In some embodiments, the first/second targets, the first/second adapters, the first/second imaging molecules and the eraser molecule are the same as or similar to those as described elsewhere herein, such as in the “Imaging Method” section.

In some embodiments, the computer is programed such that the device does not remove the liquid applied in operation (a) before performing operations (c) and (d).

In some embodiments, the first signal and the second signal overlap with each other or are identical.

In some embodiments, the device further includes a reservoir for storing the one or more targets, the first adapter, the first imaging molecule, the eraser, the second adapter, the second imaging molecule.

In some embodiments, the device further includes at least one selected from the group consisting of the first target, the first adapter, the first imaging molecule, the eraser, the second target, the second adapter, and the second imaging molecule.

In some embodiments, the device includes the first target, the first adapter, the first imaging molecule, the eraser, the second target, the second adapter, and the second imaging molecule.

### Example 1: Prophetic Examples

Embodiments of the invention open the door to a wide range of potential applications including the following.

### Example 1-1: Spatial Omics

The journal Nature Methods featured spatial omics as highly multiplexed spatially resolved techniques as method of the year 2021 (https://www.nature.com/articles/s41592-020-01033-y). Spatial omics approaches (MERFISH, SeqFISH+) are at the forefront of imaging. Xiaowei Zhuang's (from Harvard University) lab has invented multiplexed-error robust fluorescence in situ hybridization (MERFISH), which allows looking at hundreds of transcriptional RNA molecules or hundreds of genomic loci (https://www.science.org/doi/10.1126/science.aaa6090). Long Cai (from Caltech) has pioneered SeqFISH+, which allows looking at tens of thousands of different RNA species within a cell.

Both approaches are mostly relying on a diffraction-limited readout (https://www.nature.com/articles/s41586-020-03126-2). They could also be combined with STORM or DNA-PAINT.

Embodiments of the invention provide at least two advantages. First, since the adapter and the Imager are binding transient, this should decrease the washing time (time in between imaging significantly). Second, since the readout is not diffraction-limited anymore, it can be used for highly dense targets within a cell. This could, for example, enable spatial proteomics with a conventional fluorescence microscope.

### Example 1-2: In Vitro Transcriptional Assays

Highly multiplexed fluorogenic-PAINT can also be used in the context of in vitro transcription assays. Since the probes are fluorogenic, the complexity of illumination and downstream the design of a device can be drastically reduced. Additionally, the super-resolution readout can increase the throughput by increasing the surface density. In combination with the fast-imaging probes and the fast washing (due to the transient binding of adapter and Imager), this will significantly enhance the speed and throughput for this type of assays.

### Cost Reduction

A simple DNA strand in the length regime of an adapter or an Imager cost ˜10 USD at IDT (DNA synthesis company). A fluorescently labeled DNA strand costs around 200-500 USD. A fluorogenic imager carrying a fluorescent molecule on the one end and a quencher molecule on the other end costs ˜1000 USD. With the multiplexed adapter strategy, embodiments of the invention can scale the cost of an experiment/assay down of a factor of 20-100.

### Example 2: Unraveling Cellular Complexity with Unlimited Multiplexed Super-Resolution Imaging

Mapping the intricate spatial relationships between the many different molecules inside a cell is essential to understanding cellular functions in all their complexity. Super-resolution fluorescence microscopy offers the required spatial resolution but struggles to reveal more than four different targets simultaneously. Exchanging labels in subsequent imaging rounds for multiplexed imaging extends this number but is limited by its low throughput. Here the present study presents a novel imaging method for rapid multiplexed super-resolution microscopy of a nearly unlimited number of molecular targets by leveraging fluorogenic labeling in conjunction with Transient Adapter-mediated switching for high-throughput DNA-PAINT (FLASH-PAINT). The present study demonstrates the cell biological versatility of FLASH-PAINT in mammalian cells in four applications: i) mapping nine proteins in a single mammalian cell, ii) elucidating the functional organization of primary cilia by nine-target imaging, iii) revealing the changes in proximity of twelve different targets in unperturbed and dissociated Golgi stacks and iv) investigating inter-organelle contacts at 3D super-resolution.

### Example 2-1

Understanding cellular function is intimately tied to the ability to visualize how organelles and the molecules constituting them respond to diverse physiological and disease states. Meaningful, information-rich visualization is, however, a challenge as it depends on both of the abilities to identify molecules, in particular proteins, and their many interaction partners, and resolve their spatial organization. Fluorescence light microscopy has long been key here, revealing specific proteins at hundreds of nanometers resolution, or, with the advent of optical super-resolution microscopy at tens of nanometers or even sub-ten nanometer resolution. Among the different super-resolution microscopy modalities, single-molecule localization microscopy (SMLM) is a preferred choice for cell biological investigations due to its high 3D resolution (usually ˜20-70 nm), sensitivity (single molecules), and relatively low instrumentational requirements. In SMLM, single molecules spontaneously switch between ‘ON’ (bright) and ‘OFF’ (dark) states and super-resolved images are built up by computationally localizing individual ON molecules over thousands of camera frames. In contrast to SMLM techniques such as (F) PALM and (d) STORM that rely on photophysical switching between bright and dark fluorescent states, DNA-PAINT utilizes the transient reversible binding of fluorescently tagged short oligonucleotide strands, called ‘Imagers’ (or ‘Imager probes’), to complementary ‘docking strands’ that are linked to targets of interest (e.g. proteins usually tagged via antibodies) (FIG. 13A, left part). In conventional DNA-PAINT there is no true dark fluorescent state, rather the ‘OFF’ state relies on unbound Imagers being blurred to a homogeneous background by their rapid diffusion, thereby preventing their localization; only when an Imager binds to the docking strand, the transiently immobile Imager is observed as a discrete fluorescent spot (‘ON’) that can be localized. Freed of the constraints of photophysical switching, dyes and buffers in DNA-PAINT can be selected for maximum brightness. Additionally, as there is a large reserve pool of Imagers in solution, even as a bound Imager probe bleaches it can be replaced. This allows for higher densities of localization events in the final image, an otherwise limiting factor, especially when imaging volumes as thick as a cell. The combination of these advantages allows for <5 nm resolution.

While DNA-PAINT and other super-resolution techniques feature an impressive resolution improvement of a factor of ten or more over conventional fluorescence microscopy, its impact on biomedical research has been limited by a lack of multicolor imaging techniques which are instrumental to decode the intricate organization of the cell at the molecular level. The mammalian Golgi complex, for example, is organized in stacks of multiple cisternae arranged cis-to-trans. These stacks are usually connected laterally forming a highly convoluted ‘ribbon’. The complex role and structure of the Golgi and its interactions with the trans-Golgi network (TGN), endoplasmic reticulum (ER) exit sites (ERES), the ER Golgi Intermediate Compartment (ERGIC) and many other organelles is mediated by more than one thousand different proteins which interact in a selective, well-orchestrated manner as governed by their specific spatial distributions. While there is a prototypical “textbook” Golgi, the Golgi ribbon in reality varies dramatically in shape and orientation from cell to cell. This variability makes it impossible to combine individual, independently recorded super-resolution images of different subsets of two or three different proteins into a comprehensive ten or more color image that would cover more than just a small facet of the Golgi's role in cell biology.

Multicolor SMLM has traditionally been constrained by the limited availability of bright, spectrally distinguishable probes. As a result, two-color imaging has been the standard in SMLM, with three or four colors being the exception. Multiplexing approaches in which different labels are imaged sequentially, offer an avenue to overcome this limitation and have, for example, been demonstrated to extend diffraction-limited multicolor fluorescence imaging up to ˜100 labels. In super-resolution microscopy, multiplexing has been realized by the DNA-PAINT variant Exchange-PAINT. Here, different targets are labeled with orthogonal ssDNA docking strands and then imaged sequentially using different Imager probes. However, the Imager probes used so far feature slow binding kinetics which result in data acquisition of an hour or more per color channel. Adding time for washing between sequential imaging cycles, total data acquisition times typically accumulate to several days for a single cell.

The recent development of speed-optimized and fluorogenic Imager probes which allow up ˜100-fold faster imaging in DNA-PAINT, are at a first glance a solution to this severe limitation of throughput. However, due to DNA sequence design constraints of these specialized probes only six speed-optimized probes and two fluorogenic probes have so far been found. This limits the prospect of fast Exchange-PAINT to only a hand full of targets. Additionally, requiring a specific Imager probe for each target does not scale well to tens, hundreds or even thousands of probes since dye-conjugated oligonucleotides are expensive and probe exchange by extensive washing after each imaging cycle is time consuming and in accumulation damages the sample.

Here, the present study introduces fluorogenic labeling in conjunction with Transient Adapter-mediated switching for high-throughput DNA-PAINT (FLASH-PAINT), a method that allows for rapid, essentially unlimited multiplexing in super-resolution imaging. Using orthogonal ssDNA-based adapters that direct any Imager probe (e.g. a speed-optimized or fluorogenic one) to a specific target selected from a complementary set of docking strands (FIG. 13A) eliminates the color-limitation of super-resolution microscopy. Key to the success of FLASH-PAINT is that the adapters bind only transiently to the docking strands. This allows for fast, efficient, and gentle exchange of adapters between imaging cycles. Furthermore, it enables the introduction of ‘Erasers’, oligonucleotides that are complementary to individual Transient Adapters. Hybridized to any Transient Adapter of choice, these Erasers neutralize it in a highly efficient manner and thereby eliminate the need for any washing steps.

The present study demonstrates the broad utility of FLASH-PAINT by mapping spatial distributions of nine different proteins across a U-2 OS cell and revealing the complex spatial arrangement of nine proteins on individual primary cilia and twelve Golgi-related proteins in single cells. Additionally, the present study characterizes the number and size of contacts between the ER, mitochondria, lysosomes, and the Golgi complex at 3D super-resolution.

### Example 2-2: Adapter Design

Minimal crosstalk between targets is a key requirement for successful multiplexed imaging. Multiplexing approaches therefore traditionally have put emphasis on efficiently erasing previous rounds of fluorescent labels (e.g., by photobleaching, UV-cleaving or chemical stripping) before imaging the next round of labels. This difficult process of removing labels stands in stark contrast to the close to 100% dissociation efficiency of Imagers from docking sites in DNA-PAINT which is facilitated simply by the transient nature of Imager-docking site association (˜1 s). It was hypothesized that this same principle of transient binding can be applied to adapters that bind only transiently to their target. The conceptional challenge with this approach is that such an adapter will inevitably be bound to the target only for a fraction of the time and thereby reduce the overall binding frequency of Imager probes to the docking sites as compared to the conventional adapter-less DNA-PAINT approach. Importantly, however, the Transient Adapter itself is not fluorescent and therefore can be used at concentrations orders of magnitude higher (e.g., c_{TA}=10 nM-100 nM) than an Imager strand in a conventional DNA-PAINT experiment: for example, at a 50-nM Transient Adapter concentration, an average binding time of 100 s, and an association rate of 2×10⁶ M⁻¹s⁻¹, ˜91% of docking sites are occupied by an adapter.

The present study designed a set of Transient Adapters, where each adapter consisted of two binding motifs, one to an Imager probe and the other one to a docking sequence, separated by a short 2-nucleotide (nt) spacer. As Imager probe motifs, the present study selected three previously published sequences: a conventional DNA-PAINT Imager, a speed-optimized Imager and a fluorogenic Imager (Table 1). To realize the targeted dissociation rate of the order of 0.01 s⁻¹, the present study designed 12 orthogonal 10-nt motifs (Tables 2-3) with a GC content of 40%-50%.

TABLE 1

| Imager name | Sequence | 5′-mod | 3′-mod |
|---|---|---|---|
| R2-6 nt | TGGTGG | Cy3B | |
| | (SEQ ID NO: 1) | | |
| P1-9 nt | TAGATGTAT | Cy3B | |
| | (SEQ ID NO: 2) | | |
| FP2 | AAGAAGTAAAGGGAG | Cy3B | BHQ2 |
| | (SEQ ID NO: 3) | | |
| P3 | TAATGAAGA | | Cy3B |
| | (SEQ ID NO: 4) | | |
| P5 | ATACATTGA | | Cy3B |
| | (SEQ ID NO: 5) | | |
| PS3 | TCCTCCC | | Cy3B |
| | (SEQ ID NO: 6) | | |
| R3 | GAGAGAG | | Cy3B |
| | (SEQ ID NO: 7) | | |
| R4 | TGTGTGT | | Cy3B |
| | (SEQ ID NO: 8) | | |

TABLE 2

| Paper | Working | |
|---|---|---|
| ID | ID | Sequence |
| A1 | A3 | TT TCTTCATTAGCG |
| | | (SEQ ID NO: 9) |
| A2 | A19 | TT ATAAAGTGTCCA |
| | | (SEQ ID NO: 10) |
| A3 | A5 | TT TCAATGTATGGC |
| | | (SEQ ID NO: 11) |
| A4 | A10 | TT ATAATGGATGGG |
| | | (SEQ ID NO: 12) |
| A5 | A27 | TT AAAAAGTTCGAG |
| | | (SEQ ID NO: 13) |
| A6 | A36 | TT ATAACAGAATCG |
| | | (SEQ ID NO: 14) |
| A7 | A39 | TT TTATGTTCTGCT |
| | | (SEQ ID NO: 15) |
| A8 | A15 | TT ATAGTGATTGGA |
| | | (SEQ ID NO: 16) |
| A9 | A38 | TT ATTTAGTGTAGC |
| | | (SEQ ID NO: 17) |
| A10 | A8 | TT ATGTTAATGGGT |
| | | (SEQ ID NO: 18) |
| A11 | A25 | TT ATAATCATGCTC |
| | | (SEQ ID NO: 19) |
| A12 | A20 | TT ATATGATCTCCG |
| | | (SEQ ID NO: 20) |
| P1 | P1 | TT ATA CAT CTA (SEQ ID |
| | | NO: 21) |
| P3 | P3 | TT TCT TCA TTA (SEQ ID |
| | | NO: 22) |
| P5 | P5 | TT TCA ATG TAT (SEQ ID |
| | | NO: 23) |
| PS3 | PS3 | AA GGGAGGA (SEQ ID |
| | | NO: 24) |
| 5xR3 | 5xR3 | TT |
| | | TCCTCTCTCTCTCTCTC |
| | | (SEQ ID NO: 25) |
| 5xR4 | 5xR4 | TT |
| | | CAACACACACACACACA |
| | | (SEQ ID NO: 26) |

TABLE 3

| Paper ID | Working ID | Imager-DS | Spacer | Adapter-DS |
|---|---|---|---|---|
| A1-FP2 | A3-FP2 | CCTCGCTGAACCCCTTA | AA | CGCTAATGAA |
| | | (SEQ ID NO: 27) | | (SEQ ID NO: 28) |
| A2-FP2 | A19-FP2 | CCTCGCTGAACCCCTTA | AA | TGGACACTTT |
| | | (SEQ ID NO: 29) | | (SEQ ID NO: 30) |
| A3-FP2 | A5-FP2 | CCTCGCTGAACCCCTTA | AA | GCCATACATT |
| | | (SEQ ID NO: 31) | | (SEQ ID NO: 32) |
| A4-FP2 | A10-FP2 | CCTCGCTGAACCCCTTA | AA | CCCATCCATT |
| | | (SEQ ID NO: 33) | | (SEQ ID NO: 34) |
| A5-FP2 | A27-FP2 | CCTCGCTGAACCCCTTA | AA | CTCGAACTTT |
| | | (SEQ ID NO: 35) | | (SEQ ID NO: 36) |
| A6-FP2 | A36-FP2 | CCTCGCTGAACCCCTTA | AA | CGATTCTGTT |
| | | (SEQ ID NO: 37) | | (SEQ ID NO: 38) |
| A7-FP2 | A39-FP2 | CCTCGCTGAACCCCTTA | AA | AGCAGAACAT |
| | | (SEQ ID NO: 39) | | (SEQ ID NO: 40) |
| A8-FP2 | A15-FP2 | CCTCGCTGAACCCCTTA | AA | TCCAATCACT |
| | | (SEQ ID NO: 41) | | (SEQ ID NO: 42) |
| A9-FP2 | A38-FP2 | CCTCGCTGAACCCCTTA | AA | GCTACACTAA |
| | | (SEQ ID NO: 43) | | (SEQ ID NO: 44) |
| A10-FP2 | A8-FP2 | CCTCGCTGAACCCCTTA | AA | ACCCATTAAC |
| | | (SEQ ID NO: 45) | | (SEQ ID NO: 46) |
| A11-FP2 | A25-FP2 | CCTCGCTGAACCCCTTA | AA | GAGCATGATT |
| | | (SEQ ID NO: 47) | | (SEQ ID NO: 48) |
| A12-FP2 | A20-FP2 | CCTCGCTGAACCCCTTA | AA | CGGAGATCAT |
| | | (SEQ ID NO: 49) | | (SEQ ID NO: 50) |
| A1-P1 | A3-P1 | ATACATCTA (SEQ ID | TT | CGCTAATGAA |
| | | NO: 51) | | (SEQ ID NO: 52) |
| A2-P1 | A19-P1 | ATACATCTA (SEQ ID | TT | TGGACACTTT |
| | | NO: 53) | | (SEQ ID NO: 54) |
| A3-P1 | A5-P1 | ATACATCTA (SEQ ID | TT | GCCATACATT |
| | | NO: 55) | | (SEQ ID NO: 56) |
| A4-P1 | A10-P1 | ATACATCTA (SEQ ID | TT | CCCATCCATT |
| | | NO: 57) | | (SEQ ID NO: 58) |
| A5-P1 | A27-P1 | ATACATCTA (SEQ ID | TT | CTCGAACTTT |
| | | NO: 59) | | (SEQ ID NO: 60) |
| A6-P1 | A36-P1 | ATACATCTA (SEQ ID | TT | CGATTCTGTT |
| | | NO: 61) | | (SEQ ID NO: 62) |
| A7-P1 | A39-P1 | ATACATCTA (SEQ ID | TT | AGCAGAACAT |
| | | NO: 63) | | (SEQ ID NO: 64) |
| A8-P1 | A15-P1 | ATACATCTA (SEQ ID | TT | TCCAATCACT |
| | | NO: 65) | | (SEQ ID NO: 66) |
| A9-P1 | A38-P1 | ATACATCTA (SEQ ID | TT | GCTACACTAA |
| | | NO: 67) | | (SEQ ID NO: 68) |
| A10-P1 | A8-P1 | ATACATCTA (SEQ ID | TT | ACCCATTAAC |
| | | NO: 69) | | (SEQ ID NO: 70) |
| A11-P1 | A25-P1 | ATACATCTA (SEQ ID | TT | GAGCATGATT |
| | | NO: 71) | | (SEQ ID NO: 72) |
| A12-P1 | A20-P1 | ATACATCTA (SEQ ID | TT | CGGAGATCAT |
| | | NO: 73) | | (SEQ ID NO: 74) |
| A1-5xR2 | A3-5xR2 | ACCACCACCACCACCACCA | AA | CGCTAATGAA |
| | | (SEQ ID NO: 75) | | (SEQ ID NO: 76) |
| A2-5xR2 | A19-5xR2 | ACCACCACCACCACCACCA | AA | TGGACACTTT |
| | | (SEQ ID NO: 77) | | (SEQ ID NO: 78) |
| A3-5xR2 | A5-5xR2 | ACCACCACCACCACCACCA | AA | GCCATACATT |
| | | (SEQ ID NO: 79) | | (SEQ ID NO: 80) |
| A4-5xR2 | A10-5xR2 | ACCACCACCACCACCACCA | AA | CCCATCCATT |
| | | (SEQ ID NO: 81) | | (SEQ ID NO: 82) |
| A5-5xR2 | A27-5xR2 | ACCACCACCACCACCACCA | AA | CTCGAACTTT |
| | | (SEQ ID NO: 83) | | (SEQ ID NO: 84) |
| A6-5xR2 | A36-5xR2 | ACCACCACCACCACCACCA | AA | CGATTCTGTT |
| | | (SEQ ID NO: 85) | | (SEQ ID NO: 86) |
| A7-5xR2 | A39-5xR2 | ACCACCACCACCACCACCA | AA | AGCAGAACAT |
| | | (SEQ ID NO: 87) | | (SEQ ID NO: 88) |
| A8-5xR2 | A15-5xR2 | ACCACCACCACCACCACCA | AA | TCCAATCACT |
| | | (SEQ ID NO: 89) | | (SEQ ID NO: 90) |
| A9-5xR2 | A38-5xR2 | ACCACCACCACCACCACCA | AA | GCTACACTAA |
| | | (SEQ ID NO: 91) | | (SEQ ID NO: 92) |
| A10-5xR2 | A8-5xR2 | ACCACCACCACCACCACCA | AA | ACCCATTAAC |
| | | (SEQ ID NO: 93) | | (SEQ ID NO: 94) |
| A11-5xR2 | A25-5xR2 | ACCACCACCACCACCACCA | AA | GAGCATGATT |
| | | (SEQ ID NO: 95) | | (SEQ ID NO: 96) |
| A12-5xR2 | A20-5xR2 | ACCACCACCACCACCACCA | AA | CGGAGATCAT |
| | | (SEQ ID NO: 97) | | (SEQ ID NO: 98) |

### Example 2-3: Transient Adapters are Highly Specific and Bind Efficiently and Reversibly

For an initial proof of concept of FLASH-PAINT, the present study used DNA origami nanostructures. To directly compare adapter-mediated binding with direct binding, the present study imaged a mixture of two different DNA origami species with a SMLM instrument. One species featured binding sites for the Imager probe arranged in a rectangular frame, the other species featured adapter docking sites arranged in a 3×4 grid with a 20-nm spacing (FIGS. 13A-13B). In the first round of imaging, the present study introduced only the Imager strand. As expected, the present study could only observe the first species since the Imager should not bind to the adapter docking site on the second origami species. In the second round, the present study introduced the adapter along with the Imager and consequently both DNA origami species were visible. For the third round of imaging, to test the adapter dissociation efficiency, the present study washed out the mix of adapter and Imager strands, and then reintroduced the Imager only. The resulting image resembled the first image, confirming excellent dissociation efficiency. Counting how many Imager probe binding events were registered in the three images, confirmed that unspecific binding of the Imager probe to the adapter docking site lies below 1% (FIG. 13B; 0-3 vs. 316-457 events) and adapter dissociation is more than 99% efficient (0-2 events after washing).

Next, the present study designed an experiment to compare the association rate of adapter-mediated binding to the association rate of direct binding (FIG. 13C). The present study again mixed two different DNA origami species, one species featuring a single docking site for adapter-mediated binding, the other one having a direct binding site. To distinguish the two DNA origamis from each other, the present study used orthogonal docking sites arranged in a rectangle framing the single docking site and imaged these frames sequentially in two additional rounds (FIGS. 19A-191 and 20). The present study then measured the association rates of Imagers binding to the two species of DNA origamis for a constant Imager concentration (10 nM) but different adapter concentrations (FIGS. 13D and 21A-21C) using a speed Imager. In the low adapter-concentration regime, the association rate of Imagers binding to DNA origamis via adapters increases when raising the adapter concentration. This can be explained by the increase in occupancy of the docking sites by adapter strands. Beyond ˜20 nM adapter strand concentration, the association rate decreases, however. A similar decrease can simultaneously be observed in the association rate of the Imagers binding directly to the second DNA origami species and is consistent with a decrease of the available Imager concentration. This can be explained by high concentrations of adapter strands in solution competing for Imagers and thereby depleting the pool of Imagers available to bind to DNA origamis. An analytical description of the direct and adapter-mediated association rates is provided in the supplementary information and matches the experimental data points very well (curve vs. data points in FIG. 13D). Importantly, the adapter-mediated association rate of Imagers binding to DNA origamis reached with ˜70% of the association rate for direct binding (c_{TA}=0 nM) a level comparable to DNA-PAINT relying on direct, adapter-less binding of Imagers. This shows that the introduction of Transient Adapters is possible without substantially compromising the association of Imagers to the targets.

Next, the present study measured the association and dissociation rates for 36 designed adapters (Table 4), 12 each for speed (adapter concentration at 20 nM) (FIGS. 22A-22B and 23A-23B), classical (FIGS. 24A-24B & 24A-24B) and fluorogenic (FIGS. 26A-26B & 27A-27B) Imagers. In all measured cases the present study found that the association rates of the adapter-mediated binding were in a range similar to the direct binding case. This confirmed that transient adapters can generally be used without substantially compromising the association of Imagers to the targets.

TABLE 4

| | | Association | Association | Dissoci- | Dissoci- |
|---|---|---|---|---|---|
| | | Rate | Rate | ation | ation |
| | Work- | (Adapter) | (Direct) | Rate | Rate |
| Paper | ing | *10⁶ | *10⁶ | (Adapter) | (Direct) |
| ID | ID | (M⁻¹s⁻¹) | (M⁻¹s⁻¹) | (1/s) | (1/s) |
| A1-FP2 | A3-FP2 | 0.68 | 0.8 | 12.5 | 11.1 |
| A2-FP2 | A19-FP2 | 0.53 | 0.57 | 7.14 | 8.33 |
| A3-FP2 | A5-FP2 | 0.55 | 0.8 | 8.33 | 9.1 |
| A4-FP2 | A10-FP2 | 0.59 | 0.51 | 7.69 | 11.1 |
| A5-FP2 | A27-FP2 | 0.53 | 0.49 | 7.69 | 10 |
| A6-FP2 | A36-FP2 | 0.45 | 0.46 | 6.67 | 12.5 |
| A7-FP2 | A39-FP2 | 0.35 | 0.4 | 5.55 | 5.88 |
| A8-FP2 | A15-FP2 | 0.55 | 0.79 | 8.33 | 7.14 |
| A9-FP2 | A38-FP2 | 0.4 | 0.6 | 7.69 | 10 |
| A10- | A8-FP2 | 0.45 | 0.62 | 9.1 | 8.33 |
| FP2 | | | | | |
| A11- | A25-FP2 | 0.4 | 0.7 | 5.55 | 9.1 |
| FP2 | | | | | |
| A12- | A20-FP2 | 0.4 | 0.57 | 6.49 | 10 |
| FP2 | | | | | |
| A1-P1 | A3-P1 | 1 | 1.09 | 2.04 | 1.3 |
| A2-P1 | A19-P1 | 1.4 | 1.42 | 1.72 | 1.23 |
| A3-P1 | A5-P1 | 1.58 | 1.39 | 1.79 | 1.23 |
| A4-P1 | A10-P1 | 1.62 | 1.46 | 1.75 | 1.25 |
| A5-P1 | A27-P1 | 1.45 | 1.36 | 2.08 | 1.41 |
| A6-P1 | A36-P1 | 1.38 | 1.45 | 2.44 | 1.56 |
| A7-P1 | A39-P1 | 1.75 | 1.35 | 2.27 | 1.69 |
| A8-P1 | A15-P1 | 2.02 | 2.2 | 1.37 | 1 |
| A9-P1 | A38-P1 | 1.42 | 1.38 | 1.61 | 1 |
| A10-P1 | A8-P1 | 1.96 | 1.82 | 1.35 | 1 |
| A11-P1 | A25-P1 | 1.31 | 1.37 | 1.64 | 1.02 |
| A12-P1 | A20-P1 | 1.61 | 1.48 | 1.79 | 1.19 |
| A1- | A3-5xR2 | 58.33 | 53.94 | 4.76 | 5.88 |
| 5xR2 | | | | | |
| A2- | A19- | 40.95 | 46.3 | 6.25 | 6.67 |
| 5xR2 | 5xR2 | | | | |
| A3- | A5-5xR2 | 40.3 | 42.95 | 6.67 | 6.67 |
| 5xR2 | | | | | |
| A4- | A10- | 39.58 | 34.57 | 4 | 5.26 |
| 5xR2 | 5xR2 | | | | |
| A5- | A27- | 49.52 | 45.75 | 5.88 | 7.14 |
| 5xR2 | 5xR2 | | | | |
| A6- | A36- | 52.25 | 51 | 5.55 | 7.14 |
| 5xR2 | 5xR2 | | | | |
| A7- | A39- | 60.34 | 55.67 | 5.26 | 5.26 |
| 5xR2 | 5xR2 | | | | |
| A8- | A15- | 40.09 | 42.48 | 5.88 | 7.14 |
| 5xR2 | 5xR2 | | | | |
| A9- | A38- | 38.17 | 47.12 | 7.14 | 7.14 |
| 5xR2 | 5xR2 | | | | |
| A10- | A8-5xR2 | 35.36 | 39.59 | 7.69 | 7.69 |
| 5xR2 | | | | | |
| A11- | A25- | 42.53 | 41.28 | 7.14 | 7.14 |
| 5xR2 | 5xR2 | | | | |
| A12- | A20- | 47.39 | 42.64 | 5.55 | 6.67 |
| 5xR2 | 5xR2 | | | | |

### Example 2-4: Multiplexed Quantitative Super-Resolution Microscopy at High Resolution

To test multiplexed imaging via the Transient Adapters the present study designed DNA origami structures with four different orthogonal docking sites arranged to the shape of the letter's ‘Y’, ‘A’, ‘L’ and ‘E’ (FIGS. 13E & 20). The localization precision for all four rounds of imaging was ˜2 nm, which allowed us to clearly differentiate between neighboring binding sites of the letter patterns (FIGS. 13E & 28-32) and demonstrates that the use of Transient Adapters does not compromise the resolution. Using the speed-optimized Imagers, each round of imaging took ˜25 min, resulting in a total imaging time of ˜100 min.

Another unique feature of the Transient Adapters is the ability of imaging the same target of interest with different Imagers. This allowed us to compare the imaging performance of different Imagers using the same sample and imaging conditions. The outer membrane protein Tom20 in COS-7 cells was immunolabeled with antibodies featuring a ssDNA docking site and imaged under epi-illumination using speed, fluorogenic and classical Imagers via adapters (FIGS. 33A-33B). While the signal-to-noise ratio (SNR) of the fluorogenic Imager (SNR ˜30) and the speed Imager (SNR ˜40) was sufficiently high to easily distinguish bound Imagers from diffuse background, the low SNR in the classical Imager case (SNR ˜8) prevented artifact-free localization of targets in the thick sample. This demonstrates the clear advantage of the speed and fluorogenic Imager probes over the classical DNA-PAINT Imagers.

### Example 2-5: Erasers Allow for Rapid and Efficient Switching Between Adapters without Washing

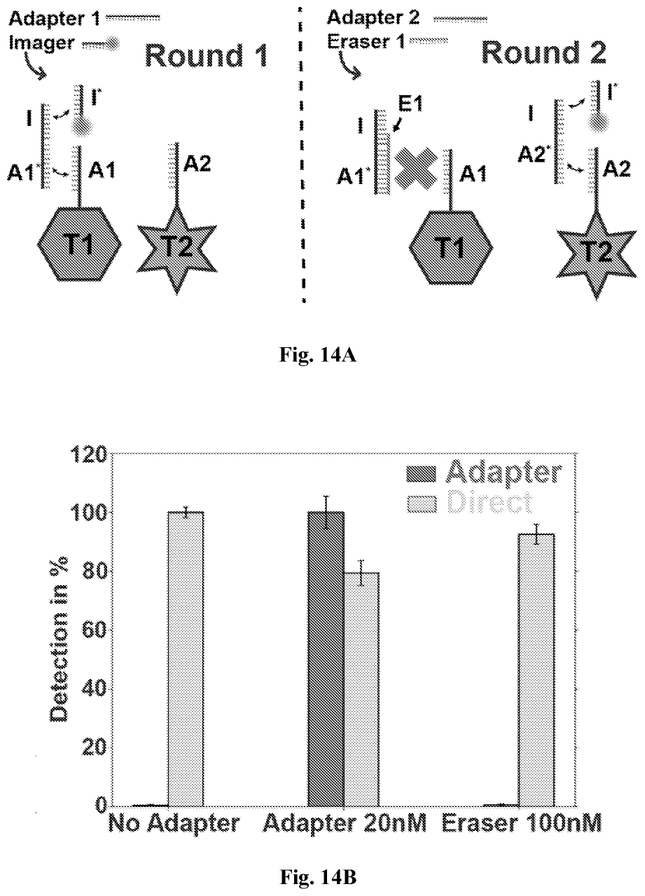

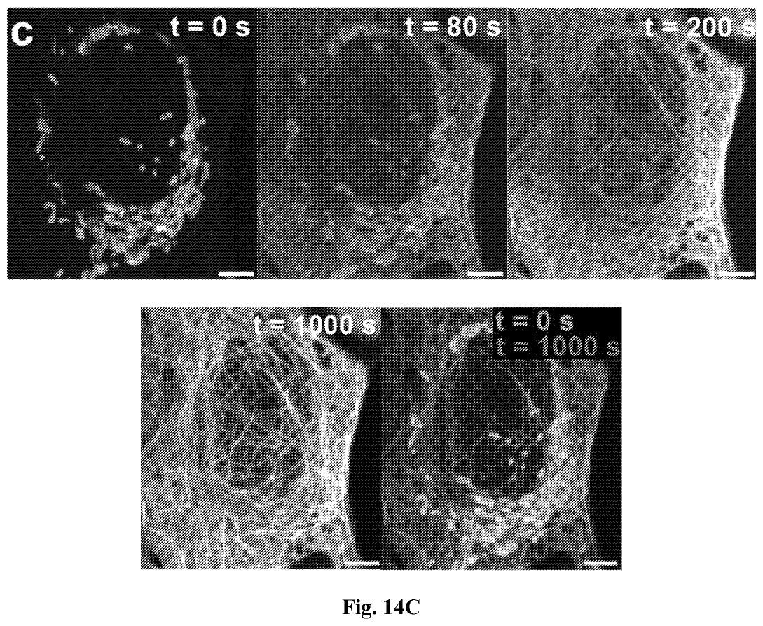

In classical Exchange-PAINT, the switch between targets is achieved by thoroughly washing out one Imager and subsequently introducing the next Imager, but this is time-consuming (typically ˜10 min). It was reasoned that the washing step could be eliminated in FLASH-PAINT by introducing an Eraser strand (FIG. 14A and Table 5) that is complementary to the Transient Adapter strand from the previous imaging round. The higher-affinity Eraser binds (effectively permanently) to the Transient Adapter and neutralizes it by preventing it from binding to the corresponding docking site.

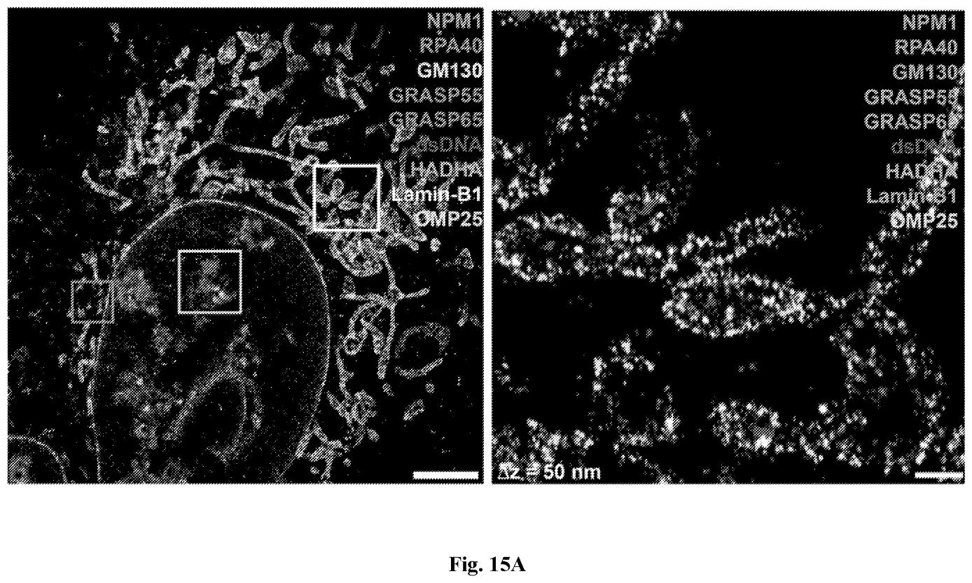

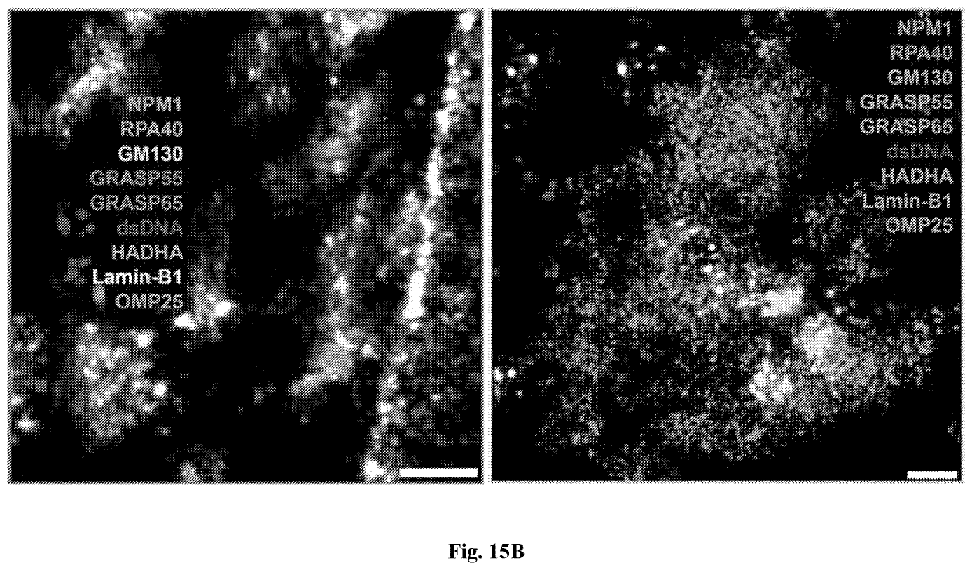

The present study characterized the erasing efficiency for all twelve Transient Adapter sequences using DNA origami structures and found it to be greater than 98% in all cases (FIGS. 14B & 44). As a test in a biological sample, the present study monitored the redirection of an Imager probe from a docking site on mitochondria (immunolabeling of the mitochondrial outer membrane protein Tom20) to a microtubule docking site (α-tubulin immunolabeling) by simultaneously introducing both an Eraser for the Transient Adapter to the mitochondria docking site and a new Transient Adapter for the microtubule docking site (FIG. 14C). Even without any flow or active perfusion, the mitochondrial signal quickly disappeared (r_{1/2}≈60 s) as the microtubule signal appeared (τ_{1/2}≈200 s) (FIG. 35A). Similarly efficient switching between two targets could be observed in the much denser environment of the cell nucleus by switching the Imager signal from the nucleolus protein NPM1 to Lamin B1 at the nuclear lamina (FIG. 15B).

To evaluate the non-specific binding of the Transient Adapters (and Imagers) for classical, speed and fluorogenic DNA-PAINT imaging in a cellular imaging context the present study imaged anti-Tom20 immunolabeled cells with matching and non-matching Transient Adapter-Imager combinations (FIGS. 36A-36D, 37A-37D & 38A-38D). It was found that in all three cases the non-specific binding was negligible (<2%) compared to the specific binding, even if eleven non-matching adapters were added.

TABLE 5

| | | | | Partial |
|---|---|---|---|---|
| | | Complement to | | complement to |
| Paper | Working | Adapter-DS | Complement | Imager-DS |
| ID | ID | sequence | to Spacer | sequence |
| E1-FP2 | E3-FP2 | TTCATTAGCG (SEQ | TT | TGG |
| | | ID NO: 99) | | |
| E2-FP2 | E19-FP2 | AAAGTGTCCA (SEQ | TT | TGG |
| | | ID NO: 100) | | |
| E3-FP2 | E5-FP2 | AATGTATGGC (SEQ | TT | TGG |
| | | ID NO: 101) | | |
| E4-FP2 | E10-FP2 | AATGGATGGG | TT | TGG |
| | | (SEQ ID NO: 102) | | |
| E5-FP2 | E27-FP2 | AAAGTTCGAG | TT | TGG |
| | | (SEQ ID NO: 103) | | |
| E6-FP2 | E36-FP2 | AACAGAATCG | TT | TGG |
| | | (SEQ ID NO: 104) | | |
| E7-FP2 | E39-FP2 | ATGTTCTGCT (SEQ | TT | TGG |
| | | ID NO: 105) | | |
| E8-FP2 | E15-FP2 | AGTGATTGGA (SEQ | TT | TGG |
| | | ID NO: 106) | | |
| E9-FP2 | E38-FP2 | TTAGTGTAGC (SEQ | TT | TGG |
| | | ID NO: 107) | | |
| E10- | E8-FP2 | GTTAATGGGT (SEQ | TT | TGG |
| FP2 | | ID NO: 108) | | |
| E11- | E25-FP2 | AATCATGCTC (SEQ | TT | TGG |
| FP2 | | ID NO: 109) | | |
| E12- | E20-FP2 | ATGATCTCCG (SEQ | TT | TGG |
| FP2 | | ID NO: 110) | | |
| E1-P1 | E3-P1 | TTCATTAGCG (SEQ | AA | TAG |
| | | ID NO: 111) | | |
| E2-P1 | E19-P1 | AAAGTGTCCA (SEQ | AA | TAG |
| | | ID NO: 112) | | |
| E3-P1 | E5-P1 | AATGTATGGC (SEQ | AA | TAG |
| | | ID NO: 113) | | |
| E4-P1 | E10-P1 | AATGGATGGG | AA | TAG |
| | | (SEQ ID NO: 114) | | |
| E5-P1 | E27-P1 | AAAGTTCGAG | AA | TAG |
| | | (SEQ ID NO: 115) | | |
| E6-P1 | E36-P1 | AACAGAATCG | AA | TAG |
| | | (SEQ ID NO: 116) | | |
| E7-P1 | E39-P1 | ATGTTCTGCT (SEQ | AA | TAG |
| | | ID NO: 117) | | |
| E8-P1 | E15-P1 | AGTGATTGGA (SEQ | AA | TAG |
| | | ID NO: 118) | | |
| E9-P1 | E38-P1 | TTAGTGTAGC (SEQ | AA | TAG |
| | | ID NO: 119) | | |
| E10-P1 | E8-P1 | GTTAATGGGT (SEQ | AA | TAG |
| | | ID NO: 120) | | |
| E11-P1 | E25-P1 | AATCATGCTC (SEQ | AA | TAG |
| | | ID NO: 121) | | |
| E12-P1 | E20-P1 | ATGATCTCCG (SEQ | AA | TAG |
| | | ID NO: 122) | | |
| E1-R2 | E3-R2 | TTCATTAGCG (SEQ | TT | TAA |
| | | ID NO: 123) | | |
| E2-R2 | E19-R2 | AAAGTGTCCA (SEQ | TT | TAA |
| | | ID NO: 124) | | |
| E3-R2 | E5-R2 | AATGTATGGC (SEQ | TT | TAA |
| | | ID NO: 125) | | |
| E4-R2 | E10-R2 | AATGGATGGG | TT | TAA |
| | | (SEQ ID NO: 126) | | |
| E5-R2 | E27-R2 | AAAGTTCGAG | TT | TAA |
| | | (SEQ ID NO: 127) | | |
| E6-R2 | E36-R2 | AACAGAATCG | TT | TAA |
| | | (SEQ ID NO: 128) | | |
| E7-R2 | E39-R2 | ATGTTCTGCT (SEQ | TT | TAA |
| | | ID NO: 129) | | |
| E8-R2 | E15-R2 | AGTGATTGGA (SEQ | TT | TAA |
| | | ID NO: 130) | | |
| E9-R2 | E38-R2 | TTAGTGTAGC (SEQ | TT | TAA |
| | | ID NO: 131) | | |
| E10-R2 | E8-R2 | GTTAATGGGT (SEQ | TT | TAA |
| | | ID NO: 132) | | |
| E11-R2 | E25-R2 | AATCATGCTC (SEQ | TT | TAA |
| | | ID NO: 133) | | |
| E12-R2 | E20-R2 | ATGATCTCCG (SEQ | TT | TAA |
| | | ID NO: 134) | | |

### Example 2-6: FLASH-PAINT Enables Spectrally Unlimited Multiplexed Super-Resolution Microscopy in Cells

To test FLASH-PAINT's capability for fast, efficient, spectrally unlimited multiplexed super-resolution microscopy, the present study imaged nine immunolabeled targets in a U2OS cell including three Golgi proteins (GM130, GRASP55, GRASP65), three mitochondria-associated targets (OMP25, HADHA, dsDNA), two nucleolus-localized targets (NPM1, RPA40) and the nuclear envelope (Lamin-B1) (FIGS. 15A-15B & 39). The present study imaged one target each in nine subsequent rounds followed by imaging all targets together in a tenth round for spatial alignment. The imaging experiment was completed in only ˜3 hours, which included the time to switch between targets. The present study achieved an average localization precision of ˜11.2 nm. To broadly demonstrate the cell biological utility of FLASH-PAINT, the present study tested the new method in three other applications.

### Example 2-7:9-Plexed FLASH-PAINT Resolves the Molecular Organization in Primary Cilia

Primary cilia function as cellular antenna that not only receive signals, but potentially transmit them by releasing vesicles from their tips. Their characteristic architecture includes a core microtubule axoneme surrounded by a specialized membrane enriched in GPCRs (e.g., Smo) and a transition zone (TZ) structure near the cilia base that gates entry into this privileged domain. To obtain a comprehensive view of primary cilia, the spatial distribution of individual proteins which organize and are enriched in these sub-diffraction (<200 nm) compartments have to be combined. This task is, however, impeded by the different states (e.g., in response to stimuli, assembly and disassembly) primary cilia can exist in, which makes it difficult to combine data from different data sets.

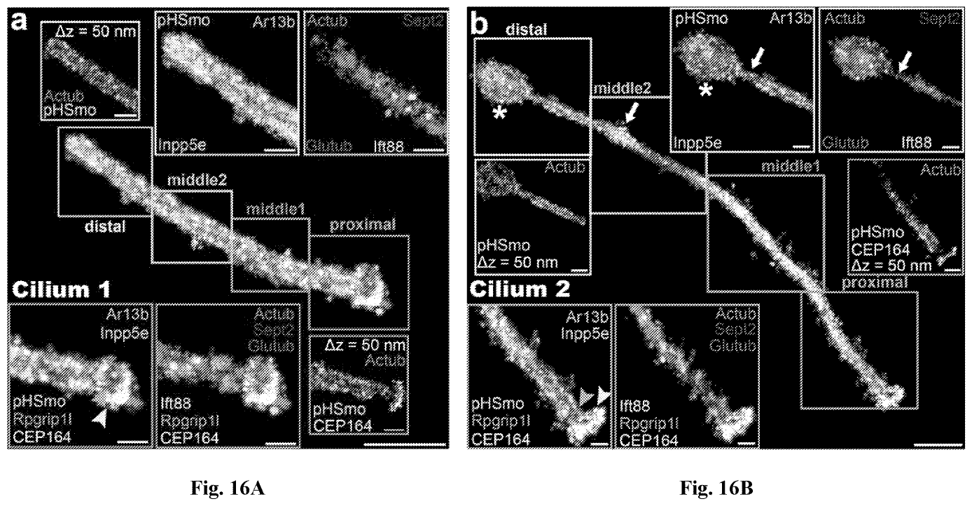

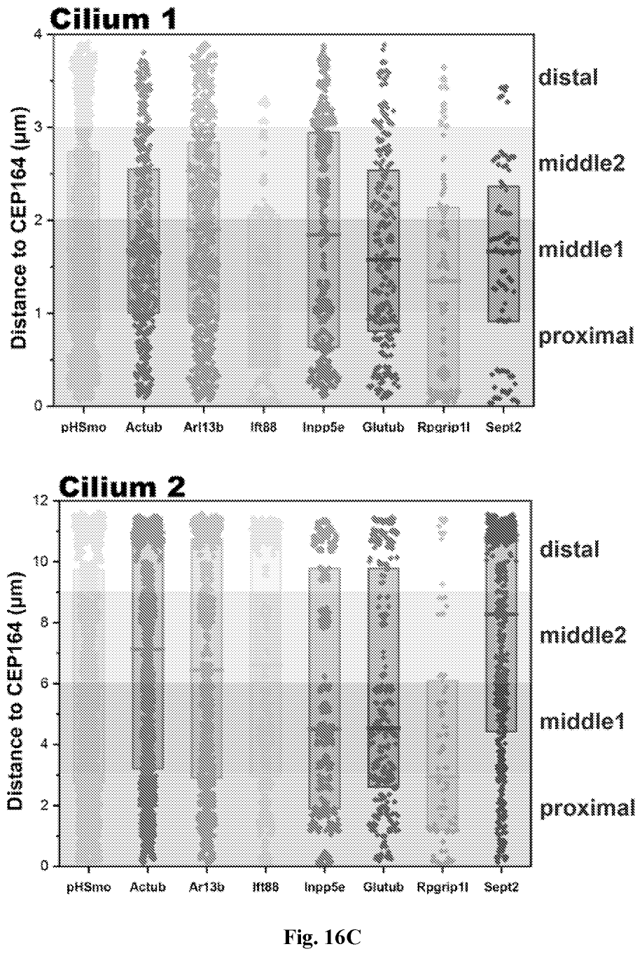

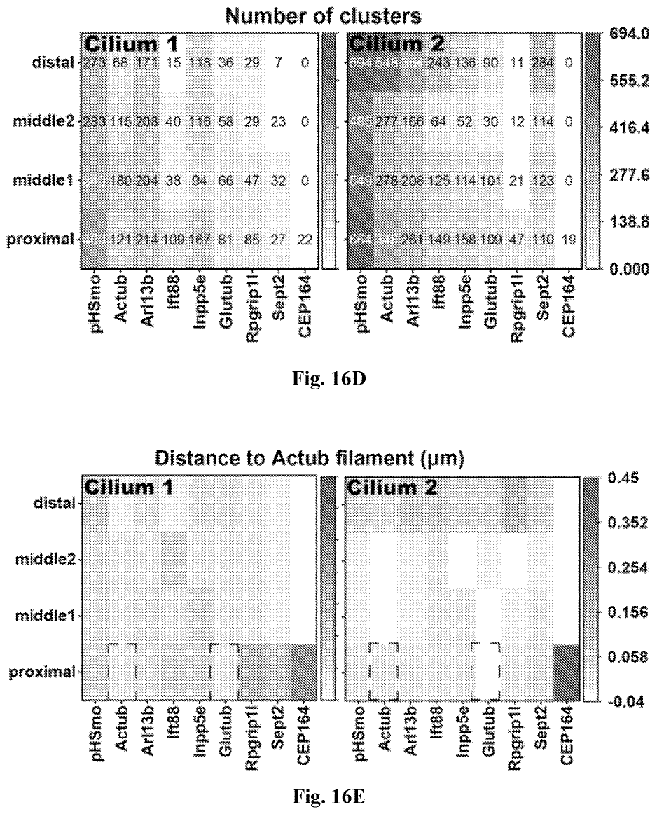

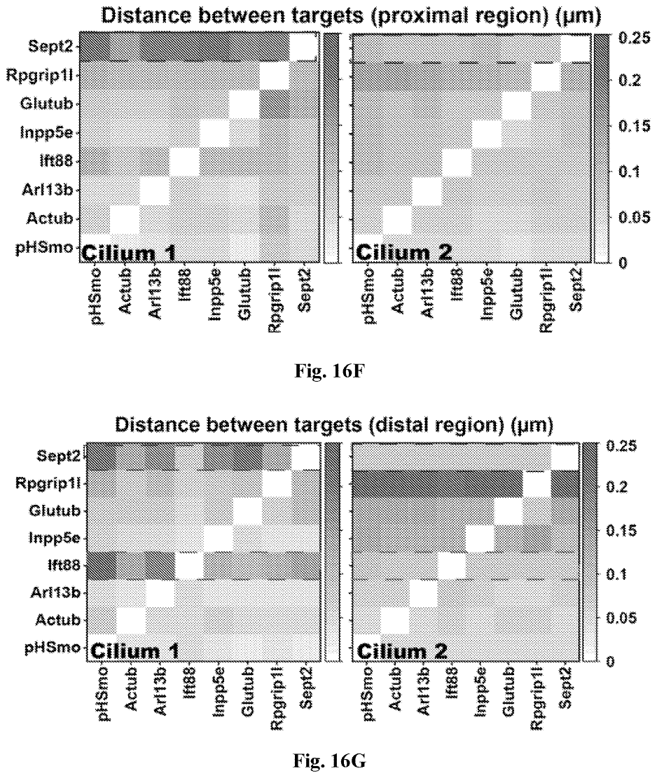

The present study tested whether FLASH-PAINT can visualize cilia nanostructure in 3D and reveal characteristic protein combinations for individual cilia compartments. To directly conjugate FLASH-PAINT docking sites to antibodies against different cilia targets, the present study utilized a Light Activated Site-Specific Conjugation (LASIC) protocol that directly conjugates the oligos to the primary antibodies. Alternatively, LASIC can be used to conjugate the oligos to secondary antibodies (FIG. 40A). The present study imaged multiple ciliated cells in 3D with 9-plex FLASH-PAINT (FIGS. 16A-16G & 40B-40C). Analyzing one cilium, the present study observed, as expected, cilium membrane proteins (pHSmo, INPP5E and Arl13b) as a tube that surrounded glutamylated and acetylated microtubules (Glu-tub and Ac-tub; yellow and blue boxes in FIGS. 16A-16B). The basal body distal appendage protein CEP164 appeared as a ring around the cilia base, with the TZ protein Rpgrip11 just distal to it (blue box, arrowheads FIGS. 16A-16B). Other cilia proteins, Sept2 and the cargo transporter Ift88 had more variable distributions. Comparing this 9-color super-resolution image with that of another cilium featuring a large bulbous tip (yellow box, asterisk, FIG. 16B) revealed striking differences: the latter cilium showed a thinning of the axoneme membrane, represented by pH-Smo, just before the bulbous tip, as well as other varicosities (arrows, FIG. 16B); interestingly, this was not apparent from the microtubule reporters, potentially as axoneme microtubule complexes can thin and become singlets as they approach the tip.

The present study next analyzed the spatial distributions along the cilia axes. The present study localized single-molecule clusters relative to the pH-Smo signal and to a central filament generated from the Ac-tub signal (FIG. 40D). In addition, since the TZ is close to the basal body, which has recently been shown to have a differential accumulation of Ac/Glu tubulin, the present study segmented the cluster data in quartiles based on their distance to CEP164 (proximal, middle1, middle2 and distal squares; FIGS. 16A-16B). Quantifying the cluster distribution in these quartiles revealed differences between the normal and bulbous cilia with Ift88 and Sept2 being enriched in the proximal and two middle regions of the normal cilium, yet recruited along the entire length of the cilium with the bulbous tip, the latter suggesting heightened activation (FIGS. 16C-16D). When the present study measured the median distances from the central filament, Ac-tub and Glu-tub showed, as expected, the lowest values (dashed rectangles in FIG. 16E). Compared to the normal cilium, the bulbous tip cilium showed a shorter distance at the proximal and middle regions for all targets (FIG. 16E). Finally, when the present study analyzed the median distances between clusters of different proteins (see Example 2-11), the present study observed that the Sept2 median distance to all other proteins was larger in the bulbous tip cilium than in the normal cilium in both the proximal and distal regions (dashed black rectangles, FIGS. 16F-16G). In contrast, for Ift88 this was only the case for the distal region (dashed purple rectangles). These findings underscore the power of multiplexed super-resolution microscopy to identify both distinct nanoscale and longer-range states in primary cilia, including rare/transient stages, that would be missed in ensemble-averaged studies of cilia.

### Example 2-8:12-Plexed FLASH-PAINT Maps the Spatial Organization of the Secretory Pathway

The present study next tested FLASH-PAINT to better visualize the complex 3D structure of the Golgi. The present study used 12-plexed super-resolution imaging to study the spatial organization of the secretory pathway by highlighting components of ER exit sites (ERES), the ER-Golgi intermediate compartment (ERGIC), cis, medial and trans cisternae of the Golgi apparatus (GA), the trans Golgi network (TGN), and COPI and COPII vesicles in the same cell. The Golgi ribbon appeared, as expected, as a highly convoluted 3D structure proximal to the nuclear lamina in HeLa cells in interphase (FIGS. 17A and 41A-41B). ERES (TANGO1), ERGIC (ERGIC-53), and COPI (β′-COP) vesicles were distributed throughout the cytoplasm (FIG. 42B). A cross section through the Golgi stack revealed the layered organization of cis-(GRASP65, GM130), medial-(ManII-GFP), and trans-cisternae (Golgin97, p230), and the trans-Golgi network (TGN46) (FIGS. 17E and 41A-41B). An en face view of the Golgi stack, revealed that Giantin localized at the rim of Golgi cisternae (FIGS. 17C and 17G), consistent with EM data. COPI (β′-COP) vesicles were observed mostly at the periphery of the Golgi ribbon (FIG. 17F), near their budding location. Most of the COPII coat (Sec31A) puncta were visibly larger than TANGO1 (ERES) puncta and usually one or more TANGO1 puncta decorated each Sec31A punctum (FIG. 17F), supporting that multiple TANGO1 proteins surround the budding sites of ERES

To visualize the 3D organization of the Golgi ribbon, the present study generated surfaces from the single-molecule localization data of GM130, ManII-GFP and Golgin97 and Lamin-B1 using a recently developed method (FIG. 17D). The median distances between the localizations of each label to those within a 500-nm range of all other labels were plotted as a heatmap (FIG. 17H). Consistent with the expected organization of the Golgi stack, this quantification showed that stack-associated proteins (GM130, GRASP65, ManII-GFP, p230, Golgin97) were closer to each other than proteins outside this group.

Imaging nocodazole-treated cells in interphase with the same labels (FIGS. 42A-42B) revealed Golgi ministacks with the cis-to-trans hierarchy (FIGS. 17I & 17M) and rim localization of Giantin (FIGS. 17K & 17O) largely intact, supporting the long-standing hypothesis that nocodazole-induced ministacks represent a valid morphological model of native Golgi. Visually comparing this data to that of the Golgi apparatus in non-treated cells, suggested that the ministacks were more proximal to ERES as marked by Sec31A and TANGO1 (FIGS. 17J & 17N). Quantifying the median distances of Golgi stack proteins to Sec31A between the two conditions showed a substantial reduction from >100 nm to the 50-nm range (FIGS. 17P and 43). This observation is consistent with the model that ER export is critical in Golgi regeneration. COPI vesicles were located at the periphery of Golgi ministacks (FIGS. 17J & 17N), as they were in non-treated cells, suggesting that vesicle budding was not affected by nocodazole. Taken together, the data both visualize and quantify for the first time the complex organization of proteins of the secretory pathway in the same cell, providing powerful morphological context with molecular specificity for future research.

### Example 2-9: FLASH-PAINT of Whole Cells Charts the Number and Size of Inter-Organelle Contact Sites

In recent years, the contact between organelles has been recognized to play critical roles in coordinating cellular function, and dysfunction of such contacts may be associated with neurodegenerative disease. Inspired by earlier work using diffraction-limited microscopy, the present study imaged four different organelles, mitochondria (Tom20), the ER (Sec61beta), the Golgi (ManII) and lysosomes (Lamp1), at 3D super-resolution in a ˜2.5-μm thick volume across a Hela cell (FIGS. 18A-18I & 44-47). For these experiments, the present study used fluorogenic Imagers, which enabled high-quality imaging of thick volumes deep in the cell in two ways: first, the fluorogenic state of the unbound state decreases the background of unbound probes in solution which is critical for large excitation volumes as used in highly inclined and laminated optical sheet (HILO) or epi-illumination. Second, the fluorogenic nature protects unbound Imagers from bleaching in solution. This is especially important for large excitation volumes since bleaching of Imagers in solution decreases the effective concentration of functional Imagers and thereby reduces the blinking frequency and data acquisition speed in a DNA-PAINT experiment.

The present study collected 41 million localizations with an average localization precision of 16.6 nm in 173 minutes. Using surface reconstruction of the localized point clouds, the present study generated 3D representations of the imaged organelles. Using the organelle surfaces the present study quantified the number of contact sites between the different organelles (FIG. 18F), defined as a spatial proximity of two membranes of <100 nm. The obtained contact site numbers were consistent with those extracted from diffraction-limited microscopy data by Valm et al. Imaging at super-resolution enabled us to additionally quantify the average area of contact sites. The present study found that the median of all contact sites was in the range between 0.1 μm² and 0.2 μm², independent of the pair of interaction partners. ER-mitochondria contact sites, which were the most abundant contacts, also showed the largest median values of their sizes and the largest size variance with about 20% of contact sites being larger than 1 μm² (FIGS. 18H & 18I).

### Example 2-10

With Transient Adapters and Erasers, the present study introduced a new concept in FLASH-PAINT that rapidly switches a fluorescent probe from one target to another. Being based on DNA technology, up to 4¹⁰, i.e. more than 1 million, designs of (10 nt-long) Transient Adapters are theoretically available-far more than the ˜20,000 different proteins expressed in a cell. While not all 1 million sequences are suitable options due to off-target binding, crosstalk, unwanted secondary structure formation and other effects, the concept yields effectively unlimited multiplexing capabilities for any currently practical proteomics study.

Importantly, the same fluorescent Imager probe (or a handful if one wants to image multiple targets simultaneously in different colors) can be used repeatedly. FLASH-PAINT can therefore leverage the newest generation of DNA-PAINT probes, that are optimizes for speed and fluorogenicity but are heavily constrained in their sequence design and are thus not directly suitable for highly multiplexed imaging. As demonstrated here, this combination enables the generation of super-resolution images of complex sub-cellular structures such as cilia or the Golgi complex at excellent quality, deep inside cells and in minutes rather than hours per imaged target.

Adapters binding stably, i.e. not transiently, have been successfully used in diffraction-limited and super-resolution microscopy. While both types of adapters enable sequential labeling of many targets with just a few fluorescent probes, stable adapters suffer from the same problem that adapter-less sequential multiplexing approaches face: previously imaged targets need to be eliminated before imaging the next one. This is usually achieved by (i) removing the adapters (using dissociation buffers or toehold-mediated displacement), (ii) permanently blocking them (with blocking strands that saturate the binding site the Imager probe normally binds to), or (iii) destroying them (with enzymes). However, all of these approaches require extensive incubation and washing periods which slow down data acquisition and can be inefficient, thereby causing crosstalk or background, and/or can damage the sample, especially if applied repeatedly over many imaging cycles.

Transient Adapters, in contrast to static adapters, by design easily dissociate from their targets without the need of toehold-mediated displacement or dissociation buffers. This fast and easy dissociation makes the sequence of the Transient Adapter that binds specifically to its target docking site readily accessible to the complementary Eraser strands. This, as the present study demonstrated (FIGS. 14B, 34A-34B, 39, 40A-40O, 41A-41B, 42A-42B), leads to highly efficient (99% to 99.8%) neutralization of the Transient Adapter. Since Erasers are specific to one particular docking site each, they do not quench the signal of other targets (in contrast to blocking strands described above that bind to the universal Imager probe binding site). Hence, neither the previous Transient Adapter nor its Eraser need to be washed out before the next imaging round. In fact, the present study can introduce the Eraser at the same time as the next Adapter (FIG. 14C & 35A-35B), which minimizes the transition time between imaging rounds.

Fully transitioning from one target to the next one required ˜1-10 minutes in the experiments (FIGS. 14C & 35A-35B). While much faster than alternative sequential multiplexing approaches that the present study did not utilize any flow chamber in the proof-of-concept experiments and were therefore limited by diffusion. The introduction of flow, combined with further optimized adapter dissociation rate constants, can reduce the transition time between targets to less than 1 minute. The present study demonstrated here up to 12-plex imaging which demonstrates broad cell biological utility of FLASH-PAINT. The primary obstacles to extending FLASH-PAINT to more targets in this study were the limited access to validated, high-quality antibodies and lacking automated microfluidics-both not of fundamental nature. While the present study focused here on immunolabeling, FLASH-PAINT will be equally useful in spatial transcriptomics studies and to trace DNA in the nucleus using fluorescence in situ hybridization. Barcoded multiplexing schemes, as demonstrated by MERFISH and SeqFISH+, allow for 1,000-fold and higher multiplexing with only tens of adapters. The low crosstalk of the Transient Adapters has the potential to minimize error rates in barcoded multiplexing. This in turn should enable researchers to use more barcodes from the codebook (i.e., barcodes with a smaller Hamming distance) and thereby provide access to more target species with fewer rounds of imaging.

It is expected that Transient Adapters will find wide application in diffraction-limited spatial omics approaches. Localization of single blinking molecules is only needed for super-resolution-if that is not required, the concentration of the Imager probe can be increased to provide diffraction-limited images as shown in FIGS. 14C & 35A-35B. In contrast to many established techniques in the field, Transient Adapters and Erasers allow for rapid exchange of labels without the use of harsh, time-consuming treatment steps, such as stripping probes off the sample or photocleaving or bleaching them, between imaging rounds. Additionally, Transient Adapters and Erasers are inexpensive: unlabeled oligos as used here cost only a fraction of their dye-labeled counterparts.

Importantly, FLASH-PAINT is not conceptually limited to imaging a single color at a time. It is anticipated that it can be readily combined with Imager probes of multiple fluorescent colors Furthermore, the technology herein synergies with innovative simultaneous multicolor approaches such as super-multiplex vibrational imaging. With synergies such as these and a broad spectrum of potential application that extends to transcriptomics and chromatin tracing, FLASH-PAINT will be an enabling technology in a wide range of biological applications.

### Example 2-11: Materials and Methods

### Materials

Unmodified DNA oligonucleotides, Cy3b-modified DNA oligonucleotides and biotinylated DNA oligonucleotides were purchased from Integrated DNA Technologies (IDT). M13mp18 scaffold (cat: N4040S) was obtained from New England BioLabs. Tris 1 M pH 8.0 (cat: AM9856), EDTA 0.5 M pH 8.0 (cat: AM9261), Magnesium 1 M (cat: AM9530G) and Sodium chloride 5 M (cat: AM9759) were obtained from Ambion. Ultrapure water (cat: 10977015) was purchased from Gibco. 200 μL PCR tubes (cat: AB-0620) were obtained from Thermo Scientific. Polyethylene glycol (PEG)-8000 (cat: 89510-250G-F) was purchased from Sigma. Streptavidin (cat: S-888) was purchased from Thermo Fisher. BSA-Biotin (cat: A8549) was obtained from Sigma-Aldrich. Tween 20 (cat: P9416-50ML), glycerol (cat: 65516-500 ml), methanol (cat: 32213-2.5L), protocatechuate 3,4-dioxygenase pseudomonas (PCD) (cat: P8279), 3,4-dihydroxybenzoic acid (PCA) (cat: 37580-25G-F) and (+−)-6-hydroxy-2,5,7,8-tetra-methylchromane-2-carboxylic acid (Trolox) (cat: 238813-5 G) were ordered from Sigma. Sodium hydroxide (cat: P3911-1 kg) was purchased from Sigma Aldrich. Potassium chloride (cat: 3624-01) was ordered from Baker Analyzed A.C.S. Reagent. 30 mL Syringes (cat: 302832) were obtained from BD. Biocompatible silicone tubing (cat: 10831), flow chambers 6-well μ-Slide VI0.5 (cat: 80607) and glass-bottomed 8-well μ-slides (cat: 80827) were obtained from ibidi. 8-wells 1.5H glass bottom chambers (cat: C8-1.5H-N) were purchased from Cellvis. 15 mL (cat: 352096) and 50 mL (cat: 352070) Polypropylene Conical Tubes and tissue culture flasks (cat: 353136) were purchased from FALCON. Dulbecco's Modified Eagle medium (DMEM) (cat: 21063-929), McCoy's 5A Medium (cat: 16600-082), Opti-MEM (cat: 31985-070), 0.05% Trypsin-EDTA (cat: 25300-054), Fetal Bovine Serum (FBS) (cat: 16000-044) and 1× Phosphate Buffered Saline (PBS) pH 7.2 (cat: 10010-023), was ordered from gibco. 10% Formalin (cat: HT501128-4L), heat inactivated FBS (cat: F4135-500ML) and 1 mg/mL fibronectin (cat: F0895-2 MG) were purchased from Sigma-Aldrich. HeLa CRM-CCL-2 cells (cat: CRM-CCL-2), U-2 OS cells (cat: HTB-96), COS-7 cells (cat: CRL-1651) and hTERT-RPE cells (cat: CRL-4000) were obtained from ATCC. Paraformaldehyde (cat: 15710) and glutaraldehyde (cat: 16219) were obtained from Electron Microscopy Sciences. Bovine serum albumin (cat: 001-000-162) was ordered from Jackson ImmunoResearch. Triton X-100 (cat: T8787-60ML) was purchased from Sigma. Antibodies against GM130 (cat: 610822), Sec31A (COPII) (cat: 612350) and p230 (cat: 611280) were obtained from BD Biosciences. Antibodies against LaminB1 (cat: ab16048), HADHA (cat: ab110302), GRASP65 (cat: ab 174834), dsDNA (cat: ab3519) and Septin2 (ab 187654) were obtained from abcam. Antibodies against GRASP55 (cat: 10598-1-AP), GM130 (cat: 11308-1-AP), TGN46 (cat: 10598-1-AP), Inpp5e (17797-1-AP), Arl13b (17711-1-AP), Ift88 (13967-1-AP), CEP164 (22227-1-AP) and, RPGRIP1L (55160-1-AP) were purchased from Proteintech. Antibodies against Tom20 (cat: sc-11415), RPA40 (cat: sc-374443) were ordered from Santa Cruz. Antibodies against NPM1 (cat: NB600-1030) were obtained from Novus Bio. Antibodies against GOLGB1 (Giantin) (cat: HPA011555), Anti-MIA3 (Tango1) (cat: HPA055922), acetylated-tubulin (T6793) and, anti-alpha-tubulin (cat: T5168) were ordered from Sigma. Antibodies against GOLGA1_1 Golgin-97 (cat: HPA044329) were purchased from Atlas Antibodies. Antibodies against LMAN1 ERGIC-53 (cat: MA5-25345) were ordered from Invitrogen. Antibodies against Glutamylated-tubulin (AB3201) were ordered from Millipore. Antibodies against Lamp1 (9091) were purchased from Cell Signaling Technology. Antibodies against mCherry (GT844 and GT857) were obtained from GeneTex. Antibodies against COPI (CMIA10) were customary made in the Rothman lab. DNA-labeled secondary anti-rabbit antibodies, DNA-labeled secondary anti-mouse antibodies and DNA-labeled GFP nanobodies were custom-ordered from Massive Photonics. Oligos conjugated to the OyOlink probe were purchased from AlphaThera.

### Buffers

Three buffers were used for sample preparation and imaging: Buffer A (10 mM Tris-HCl pH 7.5, 100 mM NaCl, 0.05% Tween 20, pH 7.5); Buffer B (10 mM MgCl₂, 5 mM Tris-HCl pH 8, 1 mM EDTA, 0.05% Tween 20, pH 7.5), and Buffer C (1×PBS, 500 mM NaCl). For the experiments shown in FIGS. 13B, 13E, 15A-15B, 17A-17P, 18A-18I, 28-32, 33A-33B, 36A-36D, 37A-37D, 38A-38D, 39, 41A-41B, 42A-42B, and 44-47, the imaging buffers were supplemented with: 1×Trolox, 1×PCA and 1×PCD.

### Trolox, PCA and PCD

100× Trolox: 100 mg Trolox, 430 μL 100% Methanol, 345 μL 1 M NaOH in 3.2 mL H₂O. 40×PCA: 154 mg PCA, 10 mL water and NaOH were mixed, and pH was adjusted to 9.0. 100×PCD: 9.3 mg PCD, 13.3 mL of buffer (100 mM Tris-HCl pH 8, 50 mM KCl, 1 mM EDTA, 50% glycerol).

### DNA Origami Self-Assembly

All DNA origami structures were designed with the Picasso design tool (see FIG. 20). Self-assembly of DNA origami was accomplished in a one-pot reaction with 50 μL total volume, consisting of 10 nM scaffold strand (sequence see Table 6), 100 nM folding staples, 10 nM (or 1 μM (FIGS. 13E and 28-32) biotinylated staples (Table 7), and 1 μM of docking site strands (List of DNA-PAINT or FLASH-PAINT handles see Table 2) in folding buffer (1×TE buffer (10 mM Tris and 1 mM EDTA) with 12.5 mM MgCl₂). The reaction mix was then subjected to a thermal annealing ramp using a thermocycler. The reaction mix was first incubated at 80° C. for 5 min, then cooled from 60 to 4° C. in steps of 1° C. every 3.21 min, and then held at 4° C.

TABLE 6
TTCCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGATTT
GGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAA
CCCTATCTCGGGCTATTCTTTTGATTTATAAGGGATTTTGCCGATTTCGGAACCACCA
TCAAACAGGATTTTCGCCTGCTGGGGCAAACCAGCGTGGACCGCTTGCTGCAACTCT
CTCAGGGCCAGGCGGTGAAGGGCAATCAGCTGTTGCCCGTCTCACTGGTGAAAAGA
AAAACCACCCTGGCGCCCAATACGCAAACCGCCTCTCCCCGCGCGTTGGCCGATTCA
TTAATGCAGCTGGCACGACAGGTTTCCCGACTGGAAAGCGGGCAGTGAGCGCAACG
CAATTAATGTGAGTTAGCTCACTCATTAGGCACCCCAGGCTTTACACTTTATGCTTCC
GGCTCGTATGTTGTGTGGAATTGTGAGCGGATAACAATTTCACACAGGAAACAGCTA
TGACCATGATTACGAATTCGAGCTCGGTACCCGGGGATCCTCTAGAGTCGACCTGCA
GGCATGCAAGCTTGGCACTGGCCGTCGTTTTACAACGTCGTGACTGGGAAAACCCTG
GCGTTACCCAACTTAATCGCCTTGCAGCACATCCCCCTTTCGCCAGCTGGCGTAATA
GCGAAGAGGCCCGCACCGATCGCCCTTCCCAACAGTTGCGCAGCCTGAATGGCGAA
TGGCGCTTTGCCTGGTTTCCGGCACCAGAAGCGGTGCCGGAAAGCTGGCTGGAGTGC
GATCTTCCTGAGGCCGATACTGTCGTCGTCCCCTCAAACTGGCAGATGCACGGTTAC
GATGCGCCCATCTACACCAACGTGACCTATCCCATTACGGTCAATCCGCCGTTTGTT
CCCACGGAGAATCCGACGGGTTGTTACTCGCTCACATTTAATGTTGATGAAAGCTGG
CTACAGGAAGGCCAGACGCGAATTATTTTTGATGGCGTTCCTATTGGTTAAAAAATG
AGCTGATTTAACAAAAATTTAATGCGAATTTTAACAAAATATTAACGTTTACAATTT
AAATATTTGCTTATACAATCTTCCTGTTTTTGGGGCTTTTCTGATTATCAACCGGGGT
ACATATGATTGACATGCTAGTTTTACGATTACCGTTCATCGATTCTCTTGTTTGCTCC
AGACTCTCAGGCAATGACCTGATAGCCTTTGTAGATCTCTCAAAAATAGCTACCCTC
TCCGGCATTAATTTATCAGCTAGAACGGTTGAATATCATATTGATGGTGATTTGACT
GTCTCCGGCCTTTCTCACCCTTTTGAATCTTTACCTACACATTACTCAGGCATTGCAT
TTAAAATATATGAGGGTTCTAAAAATTTTTATCCTTGCGTTGAAATAAAGGCTTCTCC
CGCAAAAGTATTACAGGGTCATAATGTTTTTGGTACAACCGATTTAGCTTTATGCTCT
GAGGCTTTATTGCTTAATTTTGCTAATTCTTTGCCTTGCCTGTATGATTTATTGGATGT
TAATGCTACTACTATTAGTAGAATTGATGCCACCTTTTCAGCTCGCGCCCCAAATGA
AAATATAGCTAAACAGGTTATTGACCATTTGCGAAATGTATCTAATGGTCAAACTAA
ATCTACTCGTTCGCAGAATTGGGAATCAACTGTTATATGGAATGAAACTTCCAGACA
CCGTACTTTAGTTGCATATTTAAAACATGTTGAGCTACAGCATTATATTCAGCAATTA
AGCTCTAAGCCATCCGCAAAAATGACCTCTTATCAAAAGGAGCAATTAAAGGTACT
CTCTAATCCTGACCTGTTGGAGTTTGCTTCCGGTCTGGTTCGCTTTGAAGCTCGAATT
AAAACGCGATATTTGAAGTCTTTCGGGCTTCCTCTTAATCTTTTTGATGCAATCCGCT
TTGCTTCTGACTATAATAGTCAGGGTAAAGACCTGATTTTTGATTTATGGTCATTCTC
GTTTTCTGAACTGTTTAAAGCATTTGAGGGGGATTCAATGAATATTTATGACGATTC
CGCAGTATTGGACGCTATCCAGTCTAAACATTTTACTATTACCCCCTCTGGCAAAAC
TTCTTTTGCAAAAGCCTCTCGCTATTTTGGTTTTTATCGTCGTCTGGTAAACGAGGGT
TATGATAGTGTTGCTCTTACTATGCCTCGTAATTCCTTTTGGCGTTATGTATCTGCATT
AGTTGAATGTGGTATTCCTAAATCTCAACTGATGAATCTTTCTACCTGTAATAATGTT
GTTCCGTTAGTTCGTTTTATTAACGTAGATTTTTCTTCCCAACGTCCTGACTGGTATA
ATGAGCCAGTTCTTAAAATCGCATAAGGTAATTCACAATGATTAAAGTTGAAATTAA
ACCATCTCAAGCCCAATTTACTACTCGTTCTGGTGTTTCTCGTCAGGGCAAGCCTTAT
TCACTGAATGAGCAGCTTTGTTACGTTGATTTGGGTAATGAATATCCGGTTCTTGTCA
AGATTACTCTTGATGAAGGTCAGCCAGCCTATGCGCCTGGTCTGTACACCGTTCATC
TGTCCTCTTTCAAAGTTGGTCAGTTCGGTTCCCTTATGATTGACCGTCTGCGCCTCGT
TCCGGCTAAGTAACATGGAGCAGGTCGCGGATTTCGACACAATTTATCAGGCGATG
ATACAAATCTCCGTTGTACTTTGTTTCGCGCTTGGTATAATCGCTGGGGGTCAAAGA
TGAGTGTTTTAGTGTATTCTTTTGCCTCTTTCGTTTTAGGTTGGTGCCTTCGTAGTGGC
ATTACGTATTTTACCCGTTTAATGGAAACTTCCTCATGAAAAAGTCTTTAGTCCTCAA
AGCCTCTGTAGCCGTTGCTACCCTCGTTCCGATGCTGTCTTTCGCTGCTGAGGGTGAC
GATCCCGCAAAAGCGGCCTTTAACTCCCTGCAAGCCTCAGCGACCGAATATATCGGT
TATGCGTGGGCGATGGTTGTTGTCATTGTCGGCGCAACTATCGGTATCAAGCTGTTT
AAGAAATTCACCTCGAAAGCAAGCTGATAAACCGATACAATTAAAGGCTCCTTTTG
GAGCCTTTTTTTTGGAGATTTTCAACGTGAAAAAATTATTATTCGCAATTCCTTTAGT
TGTTCCTTTCTATTCTCACTCCGCTGAAACTGTTGAAAGTTGTTTAGCAAAATCCCAT
ACAGAAAATTCATTTACTAACGTCTGGAAAGACGACAAAACTTTAGATCGTTACGCT
AACTATGAGGGCTGTCTGTGGAATGCTACAGGCGTTGTAGTTTGTACTGGTGACGAA
ACTCAGTGTTACGGTACATGGGTTCCTATTGGGCTTGCTATCCCTGAAAATGAGGGT
GGTGGCTCTGAGGGTGGCGGTTCTGAGGGTGGCGGTTCTGAGGGTGGCGGTACTAA
ACCTCCTGAGTACGGTGATACACCTATTCCGGGCTATACTTATATCAACCCTCTCGA
CGGCACTTATCCGCCTGGTACTGAGCAAAACCCCGCTAATCCTAATCCTTCTCTTGA
GGAGTCTCAGCCTCTTAATACTTTCATGTTTCAGAATAATAGGTTCCGAAATAGGCA
GGGGGCATTAACTGTTTATACGGGCACTGTTACTCAAGGCACTGACCCCGTTAAAAC
TTATTACCAGTACACTCCTGTATCATCAAAAGCCATGTATGACGCTTACTGGAACGG
TAAATTCAGAGACTGCGCTTTCCATTCTGGCTTTAATGAGGATTTATTTGTTTGTGAA
TATCAAGGCCAATCGTCTGACCTGCCTCAACCTCCTGTCAATGCTGGCGGCGGCTCT
GGTGGTGGTTCTGGTGGCGGCTCTGAGGGTGGTGGCTCTGAGGGTGGCGGTTCTGAG
GGTGGCGGCTCTGAGGGAGGCGGTTCCGGTGGTGGCTCTGGTTCCGGTGATTTTGAT
TATGAAAAGATGGCAAACGCTAATAAGGGGGCTATGACCGAAAATGCCGATGAAAA
CGCGCTACAGTCTGACGCTAAAGGCAAACTTGATTCTGTCGCTACTGATTACGGTGC
TGCTATCGATGGTTTCATTGGTGACGTTTCCGGCCTTGCTAATGGTAATGGTGCTACT
GGTGATTTTGCTGGCTCTAATTCCCAAATGGCTCAAGTCGGTGACGGTGATAATTCA
CCTTTAATGAATAATTTCCGTCAATATTTACCTTCCCTCCCTCAATCGGTTGAATGTC
GCCCTTTTGTCTTTGGCGCTGGTAAACCATATGAATTTTCTATTGATTGTGACAAAAT
AAACTTATTCCGTGGTGTCTTTGCGTTTCTTTTATATGTTGCCACCTTTATGTATGTAT
TTTCTACGTTTGCTAACATACTGCGTAATAAGGAGTCTTAATCATGCCAGTTCTTTTG
GGTATTCCGTTATTATTGCGTTTCCTCGGTTTCCTTCTGGTAACTTTGTTCGGCTATCT
GCTTACTTTTCTTAAAAAGGGCTTCGGTAAGATAGCTATTGCTATTTCATTGTTTCTT
GCTCTTATTATTGGGCTTAACTCAATTCTTGTGGGTTATCTCTCTGATATTAGCGCTC
AATTACCCTCTGACTTTGTTCAGGGTGTTCAGTTAATTCTCCCGTCTAATGCGCTTCC
CTGTTTTTATGTTATTCTCTCTGTAAAGGCTGCTATTTTCATTTTTGACGTTAAACAAA
AAATCGTTTCTTATTTGGATTGGGATAAATAATATGGCTGTTTATTTTGTAACTGGCA
AATTAGGCTCTGGAAAGACGCTCGTTAGCGTTGGTAAGATTCAGGATAAAATTGTAG
CTGGGTGCAAAATAGCAACTAATCTTGATTTAAGGCTTCAAAACCTCCCGCAAGTCG
GGAGGTTCGCTAAAACGCCTCGCGTTCTTAGAATACCGGATAAGCCTTCTATATCTG
ATTTGCTTGCTATTGGGCGCGGTAATGATTCCTACGATGAAAATAAAAACGGCTTGC
TTGTTCTCGATGAGTGCGGTACTTGGTTTAATACCCGTTCTTGGAATGATAAGGAAA
GACAGCCGATTATTGATTGGTTTCTACATGCTCGTAAATTAGGATGGGATATTATTTT
TCTTGTTCAGGACTTATCTATTGTTGATAAACAGGCGCGTTCTGCATTAGCTGAACAT
GTTGTTTATTGTCGTCGTCTGGACAGAATTACTTTACCTTTTGTCGGTACTTTATATTC
TCTTATTACTGGCTCGAAAATGCCTCTGCCTAAATTACATGTTGGCGTTGTTAAATAT
GGCGATTCTCAATTAAGCCCTACTGTTGAGCGTTGGCTTTATACTGGTAAGAATTTGT
ATAACGCATATGATACTAAACAGGCTTTTTCTAGTAATTATGATTCCGGTGTTTATTC
TTATTTAACGCCTTATTTATCACACGGTCGGTATTTCAAACCATTAAATTTAGGTCAG
AAGATGAAATTAACTAAAATATATTTGAAAAAGTTTTCTCGCGTTCTTTGTCTTGCG
ATTGGATTTGCATCAGCATTTACATATAGTTATATAACCCAACCTAAGCCGGAGGTT
AAAAAGGTAGTCTCTCAGACCTATGATTTTGATAAATTCACTATTGACTCTTCTCAGC
GTCTTAATCTAAGCTATCGCTATGTTTTCAAGGATTCTAAGGGAAAATTAATTAATA
GCGACGATTTACAGAAGCAAGGTTATTCACTCACATATATTGATTTATGTACTGTTTC
CATTAAAAAAGGTAATTCAAATGAAATTGTTAAATGTAATTAATTTTGTTTTCTTGAT
GTTTGTTTCATCATCTTCTTTTGCTCAGGTAATTGAAATGAATAATTCGCCTCTGCGC
GATTTTGTAACTTGGTATTCAAAGCAATCAGGCGAATCCGTTATTGTTTCTCCCGATG
TAAAAGGTACTGTTACTGTATATTCATCTGACGTTAAACCTGAAAATCTACGCAATT
TCTTTATTTCTGTTTTACGTGCAAATAATTTTGATATGGTAGGTTCTAACCCTTCCATT
ATTCAGAAGTATAATCCAAACAATCAGGATTATATTGATGAATTGCCATCATCTGAT
AATCAGGAATATGATGATAATTCCGCTCCTTCTGGTGGTTTCTTTGTTCCGCAAAATG
ATAATGTTACTCAAACTTTTAAAATTAATAACGTTCGGGCAAAGGATTTAATACGAG
TTGTCGAATTGTTTGTAAAGTCTAATACTTCTAAATCCTCAAATGTATTATCTATTGA
CGGCTCTAATCTATTAGTTGTTAGTGCTCCTAAAGATATTTTAGATAACCTTCCTCAA
TTCCTTTCAACTGTTGATTTGCCAACTGACCAGATATTGATTGAGGGTTTGATATTTG
AGGTTCAGCAAGGTGATGCTTTAGATTTTTCATTTGCTGCTGGCTCTCAGCGTGGCAC
TGTTGCAGGCGGTGTTAATACTGACCGCCTCACCTCTGTTTTATCTTCTGCTGGTGGT
TCGTTCGGTATTTTTAATGGCGATGTTTTAGGGCTATCAGTTCGCGCATTAAAGACTA
ATAGCCATTCAAAAATATTGTCTGTGCCACGTATTCTTACGCTTTCAGGTCAGAAGG
GTTCTATCTCTGTTGGCCAGAATGTCCCTTTTATTACTGGTCGTGTGACTGGTGAATC
TGCCAATGTAAATAATCCATTTCAGACGATTGAGCGTCAAAATGTAGGTATTTCCAT
GAGCGTTTTTCCTGTTGCAATGGCT (SEQ ID NO: 135)

TABLE 7

| Oligo Name | Sequence |
|---|---|
| Staple1-BIOTIN | ATTAAGTTTACCGAGCTCGAATTCGGGAAACCTGTCGTGC |
| | (SEQ ID NO: 136) |
| Staple2-BIOTIN | ATAAGGGAACCGGATATTCATTACGTCAGGACGTTGGGAA |
| | (SEQ ID NO: 137) |
| Staple3-BIOTIN | GCGATCGGCAATTCCACACAACAGGTGCCTAATGAGTG |
| | (SEQ ID NO: 138) |
| Staple4-BIOTIN | TTGTGTCGTGACGAGAAACACCAAATTTCAACTTTAAT |
| | (SEQ ID NO: 139) |
| Staple5-BIOTIN | ATTCATTTTTGTTTGGATTATACTAAGAAACCACCAGAAG |
| | (SEQ ID NO: 140) |
| Staple6-BIOTIN | CACCCTCAGAAACCATCGATAGCATTGAGCCATTTGGGAA |
| | (SEQ ID NO: 141) |
| Staple7-BIOTIN | AACAATAACGTAAAACAGAAATAAAAATCCTTTGCCCGAA |
| | (SEQ ID NO: 142) |
| Staple8-BIOTIN | AGCCACCACTGTAGCGCGTTTTCAAGGGAGGGAAGGTAAA |
| | (SEQ ID NO: 143) |

### DNA Origami PEG Purification

DNA origami structures featuring letters, a 10-nm and a 20-nm-grid (FIGS. 13E and 28-32) were purified via three rounds of PEG precipitation by adding the same volume of PEG-buffer (15% PEG-8000, 500 mM NaCl in 1× TE buffer, pH 8.0), centrifuging at 14,000 g at 4° C. for 30 min, removing the supernatant, and resuspending in folding buffer.

### DNA Origami Sample Preparation

For DNA origami sample preparation, a μ-Slide VI0.5 (ibidi) was used as sample chamber. First, 100 μL of biotin-labeled bovine albumin (1 mg/mL, dissolved in buffer A) were flushed into the chamber and incubated for 5 min. The chamber was then washed with 500 μL of buffer A. A volume of 100 μL of streptavidin (0.5 mg/mL, dissolved in buffer A) was then flushed through the chamber and allowed to bind for 5 min. After washing with 500 μL of buffer A and subsequently with 500 μL of buffer B, 100 μL of biotin-labeled DNA structures (˜200 μM) in buffer B were flushed into the chamber and incubated for 8 min. The chamber was then washed with 500 μL of buffer B. Finally, 100 μL of the Imager solution in the corresponding imaging buffer was flushed into the chamber.

### Plasmids

For labeling the outer membrane of mitochondria (FIGS. 15A-15B), the present study expressed GFP-OMP25 from a plasmid. For labeling medial Golgi cisternae (FIGS. 17A-17P and 18A-I), the present study expressed GFP-ManII from a plasmid. mCherry-Sec61ß was acquired from Addgene (plasmid 49155).

### Cell Culture

HeLa cells and COS-7 cells were cultured in DMEM supplemented with 10% Fetal Bovine Serum (FBS). U-2 OS cells were cultured in McCoy's 5A Medium supplemented with 10% FBS. The night before immunolabeling, cells were seeded on ibidi 8-well glass coverslips at ˜30,000 cells/well. RPE-pHSmo cells were maintained in DMEM/F12 supplemented with 10% FBS, 1× Pen/Strep, 1× non-essential amino acids and 1 mM sodium pyruvate. For ciliogenesis, 250 μL from a 50,000 cells/mL suspension of RPE-pHSmo cells were plated into 4 wells of an 8-well cellvis chamber that was coated for 1 h with 10 μg/mL fibronectin. The cells were incubated for two days at 37° C. to reach confluency. On the third day, the medium was changed to medium supplemented with 0.5% FBS to start the starvation period for another two days.

### Transient Transfection

Transfections were performed using a Super Electroporator NEPA21 Type II (Nepa Gene). Cells were concentrated to approximately 1 million cells in 90 μL in an electroporation cuvette (Bulldog Bio; 12358-346) to which 10 μL of ˜1 μg/μL of plasmid DNA were added.

Cells were electroporated using the following program: 125-V poring pulse, 3-ms pulse length, 50-ms pulse interval, two pulses, with decay rate of 10% and + polarity, followed by a 25-V transfer pulse, 50-ms pulse length, 50-ms pulse interval, five pulses, with a decay rate of 40% and #polarity.

### Golgi Ministack Induction

Golgi ministacks were induced by treating HeLa cells with 5 μg/mL of nocodazole in culture medium for 4 h at 37° C. before fixation.

### Cell Fixation and Labeling for FIGS. 14A-14C and 35A-35B

Cells were fixed with 3% PFA and 0.1% GA for 15 min. After four washes (30 s, 60 s, 2× 5 min) cells were blocked and permeabilized with 3% BSA and 0.25% Triton X-100 at room temperature for 1 h. Next, cells were incubated with primary antibodies (Table 8) in 3% BSA and 0.1% Triton X-100 at 4° C. overnight. The next day after four washes (30 s, 60 s, 2× 5 min), cells were incubated with secondary antibodies for ˜2 h at room temperature. Next, after four washes (30 s, 60 s, 2× 5 min), the sample was post-fixed with 3% PFA and 0.1% GA for 10 min. Finally, samples were washed three times with 1×PBS for 5 min each before adding the imaging solution.

TABLE 8

| # | Protein | Host | Vendor | Cat. Number | Docking site |
|---|---|---|---|---|---|
| 1 | GM130 | Rabbit | Proteintech | 11308-1-AP | N/A |
| 2 | GOLGB1 Giantin | Rabbit | Sigma | HPA011555 | N/A |
| 3 | GOLGA1_1 | Rabbit | Atlas Antibodies | HPA044329 | N/A |
| | Golgin-97 | | | | |
| 4 | TGN46 | Rabbit | Proteintech | 10598-1-AP | N/A |
| 5 | ERGIC-3 | Rabbit | Abcam | ab129179 | N/A |
| 6 | LMAN1 ERGIC-53 | Mouse | Invitrogen | MA5-25345 | N/A |
| 7 | GRASP65 | Rabbit | Abcam | ab174834 | N/A |
| 8 | Anti-MIA3 | Rabbit | Sigma | HPA055922 | N/A |
| | (Tango1) | | | | |
| 9 | p230 | Mouse | BD Biosciences | 611280 | N/A |
| 10 | COPI (CMIA10) | Mouse | Rothman Lab | Custom | N/A |
| 11 | COPII (Sec31A) | Mouse | BD | 612350 | N/A |
| 12 | LaminB1 | Rabbit | Abcam | ab16048 | N/A |
| 13 | GM130 | Mouse | BD Biosciences | 610822 | N/A |
| 14 | GRASP55 | Rabbit | Proteintech | 10598-1-AP | N/A |
| 15 | HADHA | Mouse | Abcam | ab110302 | N/A |
| 16 | dsDNA | Mouse | abcam | ab3519 | N/A |
| 17 | RPA40 | Mouse | Santa Cruz | sc-374443 | N/A |
| 18 | NPM1 | Mouse | Novus Bio | NB600-1030 | N/A |
| 19 | Tom20 | Rabbit | Santa Cruz | sc-11415 | N/A |
| 20 | Alpha-Tubulin | Mouse | Sigma | T5168 | N/A |
| 21 | Glutamylated- | Rabbit | Millipore | AB3201 | OyOlink-A8 |
| | tubulin | | | | |
| 22 | Inpp5e | Rabbit | Proteintech | 17797-1-AP | OyOlink-A10 |
| 23 | Arl13b | Rabbit | Proteintech | 17711-1-AP | OyOlink-A15 |
| 24 | Ift88 | Rabbit | Proteintech | 13967-1-AP | OyOlink-A27 |
| 25 | CEP164 | Rabbit | Proteintech | 22227-1-AP | OyOlink-A36 |
| 26 | RPGRIP1L | Rabbit | Proteintech | 55160-1-AP | OyOlink-A38 |
| 27 | Septin2 | Rabbit | Abcam | ab187654 | OyOlink-A39 |
| 28 | Acetylated-tubulin | Mouse | Sigma | T6793 | A19 |
| 29 | Anti-Lamp1 | Rabbit | Cell Signaling | 9091 | N/A |
| | | | Technology | | |
| 30 | Anti-mCherry | Mouse | GeneTex | GT844 | N/A |
| 31 | Anti-mCherry | Mouse | GeneTex | GT857 | N/A |
| 32 | GFP-Nanobody | N/A | Massive Photonics | Custom | A3 |
| 33 | Anti-Mouse | N/A | Massive Photonics | Custom | A10 |
| | Nanobody | | | | |
| 34 | Anti-Mouse | N/A | Massive Photonics | Custom | A19 |
| | Nanobody | | | | |
| 35 | Anti-Mouse | N/A | Massive Photonics | Custom | A25 |
| | Nanobody | | | | |
| 36 | Anti-Mouse | N/A | Massive Photonics | Custom | A27 |
| | Nanobody | | | | |
| 37 | Anti-Mouse | N/A | Massive Photonics | Custom | A36 |
| | Nanobody | | | | |
| 38 | Anti-Mouse | N/A | Custom | Custom | A5 |
| | Antibody | | | | |
| 39 | Anti-Rabbit | N/A | Massive Photonics | Custom | A8 |
| | Nanobody | | | | |
| 40 | Anti-Rabbit | N/A | Massive Photonics | Custom | A15 |
| | Nanobody | | | | |
| 41 | Anti-Rabbit | N/A | Massive Photonics | Custom | A20 |
| | Nanobody | | | | |
| 42 | Anti-Rabbit | N/A | Massive Photonics | Custom | A38 |
| | Nanobody | | | | |
| 43 | Anti-Rabbit | N/A | Massive Photonics | Custom | A39 |
| | Nanobody | | | | |
| 44 | Anti-Rabbit | N/A | Massive Photonics | Custom | 5xR3 |
| | Nanobody | | | | |
| 45 | Anti-Rabbit | N/A | Massive Photonics | Custom | 5xR4 |
| | Nanobody | | | | |
| 46 | Anti-Rabbit | N/A | Custom | Custom | A5 |
| | Antibody | | | | |
| 47 | Anti-Rabbit | N/A | Custom | Custom | A19 |
| | Antibody | | | | |

### Cell Fixation and Labeling for FIGS. 15A-15B and 39

Cells were fixed with 4% PFA for 1 h. After four washes (30 s, 60 s, 2× 5 min) cells were blocked and permeabilized with 3% BSA and 0.25% Triton X-100 at room temperature for 1 h. Next, cells were incubated with primary antibodies against GM-130 and LaminB1 (Table 8) in 3% BSA and 0.1% Triton X-100 at 4° C. overnight. The other primary antibodies were pre-incubated with the corresponding nanobodies (Table 8) at 4° C. overnight. The next day, after four washes (30 s, 60 s, 2× 5 min), cells were incubated with the nanobodies corresponding to anti-GM130 (host: mouse) antibody and anti-LaminB1 (host: rabbit) antibody for ˜2 h at room temperature. Next, to block unlabeled epitopes, unlabeled excess secondary nanobodies were added to pre-incubation antibody and nanobody mixes at room temperature for 5 min. Next, the cells were incubated with the pooled antibody and nanobody mix for ˜2.5 h at room temperature. After four washes (30 s, 60 s, 2× 5 min) the sample was post-fixed with 3% PFA and 0.1% GA for 10 min. Finally, samples were washed three times with 1×PBS for 5 min each before adding the imaging solution.

### Cell Fixation Preserving Cilia (FIGS. 16A-16H & 40A-40O)

After ciliogenesis induction, RPE-pHSmo cells were washed with 1×PBS and fixed with 10% Formalin for 15 min. Next, cells were washed three times with 1×PBS and permeabilized with PBS/0.1% Triton X-100 (PBST) for 10 min. Following permeabilization, the cells were washed with PBST and blocked with 3% BSA/PBST solution for 1 h. To conjugate cilia-targeted primary antibodies (Table 8) to binder oligo-OyOlink molecules, 1 μg of purified antibody was mixed with 1 μg OyOlink (1:3 molar ratio) in a total of 10 μL with PBS in a 100 μL clear PCR tube. The tubes were then incubated for 2 h on a UV transilluminator box equipped with a 365-nm excitation light source. After light-induced cross-linking, the volumes were mixed and 200 μL of 3% BSA/PBST were added. 1 μL of 2.5 M Nano-GFP A3 and 0.5 μL of mouse anti-acetylated tubulin were added to the mixture. Then 150 μL of this solution was added to one of the wells with the ciliated pHSmo cells and incubated at 4° C. overnight. The next day, the cells were washed three times with PBST for 5 min each and incubated for 2 h with anti-mouse A19 secondary antibody diluted 1:500 in blocking buffer. The sample was then washed three times with PBST for 5 min each, twice with 1×PBS and incubated for 10 min with 10% PFA and 0.1% GA. After post-fixation, the samples were washed three times with 1×PBS each and stored at 4° C. until imaging.

### Cell Fixation Preserving Golgi Complex (FIGS. 17A-17P, 41A-41B & 42A-42B)

Cells were fixed with 4% PFA for 30 min. After four washes (30 s, 60 s, 2× 5 min), cells were blocked and permeabilized with 3% BSA and 0.25% Triton X-100 at room temperature for 1 h. Next, cells were incubated with the anti-MIA3 antibody, anti-p230 antibody, and the GFP-Nanobody in 3% BSA and 0.1% Triton X-100 at 4° C. overnight. Additionally, all other primary antibodies were pre incubated with the corresponding nanobodies (Table 8) at 4° C. overnight. The next day, after four washes (30 s, 60 s, 2× 5 min) cells were incubated with the nanobodies corresponding to anti-MIA3 antibody and anti-p230 antibody for ˜2 h at room temperature. Next, unlabeled excess secondary nanobodies (to block unlabeled epitopes) were added to pre-incubation antibody-nanobody mixes at room temperature for 5 min. Next, the cells were incubated with the pooled antibody-nanobody mix for ˜2.5 h at room temperature. After four washes (30 s, 60 s, 2× 5 min), the sample was post-fixed with 3% PFA and 0.1% GA for 10 min. Finally, samples were washed three times with 1×PBS for 5 min each before adding the imaging solution.

### Cell Fixation Preserving ER, Golgi Complex, Lysosomes and Mitochondria (FIGS. 18A-18I & 44-47)

Cells were fixed with 3% PFA and 0.1% GA for 15 min. After four washes (30 s, 60 s, 2× 5 min), cells were blocked and permeabilized with 3% BSA and 0.25% Triton X-100 at room temperature for 1 h. Next, cells were incubated with primary antibodies and nanobodies (Table 8) in 3% BSA and 0.1% Triton X-100 at 4° C. overnight. The next day, cells were incubated with secondary antibodies for ˜2 h at room temperature after four washes (30 s, 60 s, 2× 5 min). Next, after four washes (30 s, 60 s, 2× 5 min), the sample was post-fixed with 3% PFA and 0.1% GA for 10 min. Finally, samples were washed three times with 1×PBS for 5 min each before adding the imaging solution.

### Cell Fixation Preserving Nuclear Lamina and Nucleoli (FIG. 35A)

Cells were fixed with 2.4% PFA for 30 min. After four washes (30 s, 60 s, 2×5 min), cells were blocked and permeabilized with 3% BSA and 0.25% Triton X-100 at room temperature for 1 h. Next, cells were incubated with primary antibodies (Table 8) in 3% BSA and 0.1% Triton X-100 at 4° C. overnight. The next day, after four washes (30 s, 60 s, 2× 5 min), cells were incubated with secondary antibodies for 2 h at room temperature. Next, after four washes (30 s, 60 s, 2× 5 min), the sample was post-fixed with 3% PFA and 0.1% GA for 10 min. Finally, samples were washed three times with 1×PBS for 5 min each before adding the imaging solution.

### Cell Fixation Preserving Mitochondria (FIGS. 33A-33B, 35A-35B, 36A-36D & 37A-37D)

Cells were fixed with 3% PFA and 0.1% GA for 15 min. After four washes (30 s, 60 s, 2× 5 min), cells were blocked and permeabilized with 3% BSA and 0.25% Triton X-100 at room temperature for 1 h. Next, cells were incubated with primary antibodies against Tom20 (Table 8) in 3% BSA and 0.1% Triton X-100 at 4° C. overnight. The next day, after four washes (30 s, 60 s, 2× 5 min), cells were incubated with secondary antibodies for 2 hours at room temperature. Next, after four washes (30 s, 60 s, 2× 5 min), the sample was post-fixed with 3% PFA and 0.1% GA for 10 min. Finally, samples were washed three times with 1×PBS for 5 min each before adding the imaging solution.

### Super-Resolution Microscope Setup

Fluorescence imaging was carried out on an inverted Nikon Eclipse Ti2 microscope (Nikon Instruments) with a Perfect Focus System, equipped with an Andor Dragonfly unit. The Dragonfly was used in the BTIRF mode, applying an objective-type TIRF or HiLo configuration with an oil-immersion objective (Nikon Instruments, Apo SR TIRF 60×, NA 1.49, Oil). For excitation, a 561-nm laser (1 W nominal laser power) was used. The beam was coupled into a multimode fiber going through the Andor Borealis unit reshaping the beam from a Gaussian profile to a homogenous flat top. As dichroic mirror, a CR-DFLY-DMQD-01 was used. Fluorescence light was spectrally filtered with an emission filter (TR-DFLY-F600-050) and imaged with a scientific complementary metal oxide semiconductor (sCMOS) camera (Sona 4BV6X, Andor Technologies) without further magnification, resulting in an effective pixel size of 108 nm. Three-dimensional super-resolution imaging was performed by introducing astigmatism via a cylindrical lens in front of the camera.

### Imaging Conditions

A high-level summary of all experiments is described in Table 9.

TABLE 9

| | Number of | | | Number of | Camera exposure | |
|---|---|---|---|---|---|---|
| | imaging | | | camera | time/readout | Laser power/ |
| FIG. | rounds | c_{imager} | c_{adapter} | frames | bandwidth | intensity |
| 13B | 1 | 500 pM | 20 nM | 10,000 | 100 ms/ | 50 mW/ |
| | | | | | 200 MHz | ~1.6 kW/cm² |
| 13C | 1 | 5 nM- | 50 nM | 5,000- | 100 ms/ | 27 mW-50 mW/ |
| | | 10 nM | | 10,000 | 200 MHz | ~0.8 kW/cm² |
| | | (P1, P3, | | | | ~1.6 kW/cm |
| | | PS3) | | | | |
| 13D & | 1 | 5 nM- | 0- | 5,000- | 33 ms-50 ms/ | 15 mW-50 mW/ |
| 21A- | | 10 nM | 100 nM | 30,000 | 200 MHz | ~0.5 kW/cm²- |
| 21C | | (R2, | | | | 1.6 kW/cm² |
| (each | | PS3, P1) | | | | |
| data | | | | | | |
| point) | | | | | | |
| 13E & | 4 | 200 pM | 20 nM | 10,000 | 100 ms/ | 90 mW/ |
| 28-32 | | (R2) | | | 200 MHz | ~2.9 kW/cm² |
| 14A- | 2 | 10 nM | 20 nM | 5,000 | 200 ms/ | 5 mW/ |
| 14C & | | (R2) | | | 200 MHz | 0.15 kW/cm² |
| 35 | | | | | | |
| 15A- | 10 | 100 pM- | 20 nM | 40,000 | 20 ms/ | 70-100 mW/ |
| 15B & | | 4 nM | | | 540 MHz | 2.3-3.3 kW/cm² |
| 39 | | (R2) | | | | |
| 16A- | 9 | 500 pM- | 20 nM | 10,000 | 20 ms/ | 50 mW |
| 16G & | | 2 nM | | per | 540 MHz | 1.6 kW/cm² |
| 40 | | (R2) | | plane | | |
| 17A- | 13 | 100 pM- | 20 nM | 30,000 | 20 ms/ | 94 mW-188 mW |
| 17P & | | 2 nM | | | 540 MHz | 3.1-6.1 kW/cm² |
| 41-42 | | (R2) | | | | |
| 18A- | 4 | 10 nM- | 50 nM | 20,000- | 10 ms/ | 125 mW/ |
| 18I & | | 40 nM | | 40,000 | 540 MHz | 4.1 kW/cm² |
| 44-47 | | (FP2) | | per | | |
| | | | | plane | | |
| 22-27 | 4 | 5 nM | 20 nM & | 5,000- | 33 ms-100 ms | 25-90 mW |
| | | (R2, | 50 nM | 30,000 | 200 Mhz/ | 0.8-1.6 kW/cm² |
| | | PS3, P1, | | | 540 MHz | |
| | | P5) | | | | |
| 44 | 6 | 5 nM | 20 nM | 5,000- | 33 ms-50 ms | 15-50 mW |
| | | (R2, | | 30,000 | 200 Mhz/ | 0.5-1.6 kW/cm² |
| | | PS3, P5) | | | 540 MHz | |
| 43 | 4 | 400 pM- | 20 nM | 20,000 | 30 ms/ | 80 mW/ |
| | | 2 nM | | | 540 MHz | 2.6 kW/cm² |
| | | (P1, R2, | | | | |
| | | FP2) | | | | |
| 36-38 | 4 | 1 nM- | 20 nM- | 30,000 | 33 ms/ | 80 mW/ |
| | | 20 nM | 50 nM | | 540 MHz | 2.6 kW/cm² |

### Image Analysis

Raw fluorescence microscopy images were subjected to spot-finding and subsequent super-resolution reconstruction, drift correction, filtering and alignment using the ‘Picasso’ software package. x, y and z drift correction were performed with a redundant cross-correlation which is integrated in the same software package. The surface reconstruction from localization data and the subsequent analysis of the contact sites were done using PYMEVisualize42. To identify and quantify clusters and distances on the cilia 9-plex data set the individual Picasso reconstructed cilium datasets were loaded into Imaris (Oxford instruments, version 10.0) to generate surfaces that were used to mask the localization data for each target at the cilium. After the surface mask was applied, localizations were processed with a Gaussian filter equivalent to one-pixel size. The filtered data was then used to generate spots using the Imaris spot detection algorithm to represent the size of the localization clusters. These spots were used to quantify the number of cluster and distances between targets. In addition, the Actub clusters were used to generate a filament representing the axoneme location along the length of the cilium.

### Example 2-12: Derivation of the Effective Association Rate of Imager Probes Binding to DNA Origami in the Presence of Transient Adapters

The observed number of transient binding events per time unit for a given DNA origami as shown in FIGS. 13C-13D, can be described by the average dark time, i.e. the time no Imager probe is bound to a DNA origami (depending on the design of the DNA origami either via a Transient Adapter or directly). These times τ_{off.TA-mediated} and τ_{off.direct} can be described as functions of the Imager probe concentration cₗₘ that was added to the imaging buffer by introducing effective association rates k_{a.eff.TA-mediated} and k_{a.eff.direct} for the Transient Adapter-mediated and the direct binding case, respectively (Definitions are summarized at the end of this derivation):

$${τ}_{off , TA - mediated} = \frac{1}{{k}_{a , eff , TA - mediated} \text{?}} \qquad ( 1 )$$

$${τ}_{off , direct} = \frac{1}{{k}_{a , eff , direct} \text{?}} \qquad ( 2 )$$

$$\text{?} \text{indicates text missing or illegible when filed}$$

The effective association rate k_{a.eff.TA-mediated} of Imager probes binding to a DNA origami docking site via Transient Adapters is influenced by both (i) the occupancy of the docking site by a Transient Adapter and (ii) the affinity between the Transient Adapter and Imager probes. The latter not only affects how efficiently Imager probes are recruited to the DNA origami, but Transient Adapters in solution also compete for these Imager probes and thereby reduce the pool of Imager probes readily available to bind to a Transient Adapter bound to the DNA origami target. This latter phenomenon also affects k_{a.eff.direct} since both DNA origami species are imaged in the same sample.

To derive k_{a.eff.TA-mediated}, two assumptions were made: an Imager probe can only bind to a docking site when a Transient Adapter strand is present. Second, only the unbound fraction of Imager probes present in the solution can bind to a docking site. k_{a.eff.TA-mediated}, can be described as the product of the Duty Cycle, D, i.e. the fraction of time a docking site is occupied by a Transient Adapter, and the association rate constant of Imager probes binding to a Transient Adapter, k_{a.lm}:

$${k}_{a , eff , TA - mediated} = D * k \text{?} \qquad ( 3 )$$

$$\text{?} \text{indicates text missing or illegible when filed}$$

The Duty Cycle can be expressed as:

$$D = \frac{\text{?}}{\text{?}} \qquad ( 4 )$$

$$\text{?} \text{indicates text missing or illegible when filed}$$

The average time τ_{off.TA} where no Transient Adapter is bound at a docking site depends on both the concentration of the Transient Adapter, c_{TA}, and the association rate constant of Transient Adapters binding to a docking site, k_{a.TA}:

$${τ}_{off , TA} = \frac{1}{\text{?}} \qquad ( 5 )$$

$$\text{?} \text{indicates text missing or illegible when filed}$$

For a 50-nM Transient Adapter concentration, an average binding time of 100 s, and an association rate of 2×10⁶ M⁻¹s⁻¹, the Duty Cycle is, for example, 91%.

As discussed above, a high concentration of Transient Adapters in solution, i.e. not bound to any docking site, will cause a non-negligible fraction of Imager probes to be bound to these Transient Adapters without producing localizable signal. Only the free fraction of unbound Imager probes, f, is available to bind to Transient Adapters bound to docking sites, reducing the concentration of Imager probes in solution below what was initially added to the imaging buffer:

$$c \text{?} = f * c \text{?} \qquad ( 6 )$$

$$\text{?} \text{indicates text missing or illegible when filed}$$

Following Jarmoskaite et al.¹, the fraction f of unbound Imager probes can be expressed as:

$$f = 1 - \frac{( \text{?} + {c}_{TA} + {K}_{D} ) - \sqrt{{( \text{?} + {c}_{TA} + {K}_{D} )}^{2} - 4 \text{?}}}{2 * \text{?}} \qquad ( 7 )$$

$$\text{?} \text{indicates text missing or illegible when filed}$$

Here, K_{D} is the equilibrium dissociation constant between Transient Adapters and Imager probes:

$${K}_{D} = \frac{{τ}_{off} \text{?}}{\text{?}} \qquad ( 8 )$$

$$\text{?} \text{indicates text missing or illegible when filed}$$

K_{D} can be estimated from measuring the average on and off times of Imager probes bound to DNA origamis featuring the same Imager probe binding site as the Transient Adapter at c_{TA}=0. Here it was assumed that these times depend only on the oligonucleotide sequence while possible effects from the surrounding environment (DNA origami vs. Transient Adapter) are negligible.

Combining Equations 3-8, the effective association rate constant of Imager probes binding to a docking site via Transient Adapters as a function of c_{TA} can be expressed as:

$${k}_{a , eff , TA - mediated} ( {c}_{TA} ) = \frac{\text{?}}{\text{?}} * ( 1 - \frac{\text{?}}{2 * \text{?}} ) * k \text{?} \qquad ( 9 )$$

$$\text{?} \text{indicates text missing or illegible when filed}$$

For comparison: the effective association rate k_{a.eff.direct} as a function of c_{TA} for Imager probes directly binding to a complementary docking site on a DNA origami in the presence of Transient Adapters in solution will equally be reduced by a factor f (i.e. Equation 6 applies) compared to k_{a.lm}, but is independent of the Duty Cycle D. It can be expressed as:

$$( 9 )$$

$${k}_{a , eff , direct} ( {c}_{TA} ) = ( 1 - \frac{( \text{?} + {c}_{TA} + \frac{\text{?}}{\text{?}} ) - \sqrt{{( \text{?} + {c}_{TA} + \frac{\text{?}}{\text{?}} )}^{2} - 4 * \text{?} * {c}_{TA}}}{2 * \text{?}} ) * k \text{?}$$

$$\text{?} \text{indicates text missing or illegible when filed}$$

The solid curves in FIG. 13D were calculated using Equations 9 and 10 and the following values:

$$c \text{?} = 10 nM$$

$${τ}_{on} \text{?} = 0.25 s$$

$${τ}_{on , TA} = 100 s$$

$$k \text{?} = 45 * {10}^{6} {M}^{- 1} {s}^{- 1}$$

$$k \text{?} = 3 * {10}^{6} {M}^{- 1} {s}^{- 1}$$

$$\text{?} \text{indicates text missing or illegible when filed}$$

The following definitions were used:
τ_{off.TA-mediated}: Average time no Imager probe is bound to a docking site of a DNA origami designed to bind Imager probes via a Transient Adapter
τ_{off.direct}: Average time no Imager probe is bound to a docking site of a DNA origami designed to directly bind Imager probes
τ_{off.TA}:Average time no Transient Adapter is bound to a docking site of a corresponding DNA origami
τ_{on.TA}: Average time a Transient Adapter is bound to a docking site of a corresponding DNA origami
τ_{off.lm}: Average time no Imager probe is bound to a specific docking site in the absence of Transient Adapters
τ_{on.lm}: Average time an Imager probe is bound to its complimentary sequence either as part of a Transient Adapter, or as a direct docking site on a corresponding DNA origami
k_{a.eff.TA-mediated}: Effective association rate constant of Imager probe binding to DNA origami docking site via a Transient Adapter, including corrections for Duty Cycle and competition from binding to Transient Adapters in solution
k_{a.eff.direct}: Effective association rate constant of Imager probe binding directly to a DNA origami featuring a suitable to docking site in the presence of Transient Adapters, including competition from binding to Transient Adapters in solution
k_{a.lm}: Association rate constant of Imager probes binding to their complimentary sequence
k_{a.TA}: Association rate constant of Transient Adapter binding to a docking site
D: Duty Cycle, i.e. fraction of time a docking site is occupied by a Transient Adapter
cᵢₘ: Molar concentration of Imager probe initially added to imaging buffer
c_{im.free}: Molar concentration of Imager probes not bound to Transient Adapters in solution
c_{TA}. Molar concentration of Transient Adapter
f: Unbound fraction of Imager probes
K_{D}: Equilibrium dissociation constant between Imager probes and Transient Adapters

### ENUMERATED EMBODIMENTS

In some aspects, the present invention is directed to the following non-limiting embodiments:

Embodiment 1: A method of microscopy imaging, the method comprising:
exposing a sample having a plurality of targets to a plurality of transient single-strand-nucleic-acid adapter molecules; and
exposing the sample to a plurality of single-strand-nucleic-acid imaging molecules; and
exposing the sample to an illumination source having a wavelength capable of interacting with the plurality of single-strand-nucleic-acid imaging molecules;
wherein the transient single-strand-nucleic-acid adapter molecules comprise:
   a first region having a target-complementary sequence; and
   a second region having a single-strand-nucleic-acid-imaging-molecule-complementary sequence.

Embodiment 2: The method of Embodiment 1, wherein the plurality of transient single-strand-nucleic-acid adapter molecules have a quantity greater than an estimated or actual quantity of targets.

Embodiment 3: The method of Embodiment 1, wherein the plurality of transient single-strand-nucleic-acid adapter molecules have a quantity or concentration greater than the plurality of single-strand-nucleic-acid imaging molecules.

Embodiment 4: The method of Embodiment 1, wherein the plurality of transient single-strand-nucleic-acid adapter molecules have a quantity or concentration greater than the plurality of single-strand-nucleic-acid imaging molecules by a ratio selected from the group consisting of: at least about 1; at least about 10; and at least about 100.

Embodiment 5: The method of Embodiment 1, wherein the plurality of transient single-strand-nucleic-acid adapter molecules have a quantity or concentration greater than the plurality of single-strand-nucleic-acid imaging molecules by a ratio of about 500.

Embodiment 6: The method of Embodiment 1, wherein the target-complementary sequence is less than 11 nucleotides.

Embodiment 7: The method of Embodiment 1, wherein the target-complementary sequence is selected from the group consisting of: between 6 and 10 nucleotides and between 8 and 10 nucleotides.

Embodiment 8: The method of Embodiment 1, further comprising:
exposing the sample to an eraser molecule adapted and configured to quench the transient single-strand-nucleic-acid adapter molecules;
exposing the sample to a second plurality of transient single-strand-nucleic-acid adapter molecules having a second, different target-complementary sequence; and
exposing the sample to the plurality of single-strand-nucleic-acid imaging molecules; and
exposing the sample to an illumination source having a wavelength capable of interacting with the plurality of single-strand-nucleic-acid imaging molecules.

Embodiment 9: The method of Embodiment 8, wherein the method is performed without rinsing the plurality of transient single-strand-nucleic-acid adapter molecules from the sample.

Embodiment 10: The method of Embodiment 8, wherein the eraser molecule and the second plurality of transient single-strand-nucleic-acid adapter molecules are introduced simultaneously.

Embodiment 11: The method of Embodiment 8, wherein the eraser molecule and the second plurality of transient single-strand-nucleic-acid adapter molecules are introduced sequentially.

Embodiment 12: The method of Embodiment 1, wherein the plurality of single-strand-nucleic-acid imaging molecules include a speed-optimized sequence.

Embodiment 13: The method of Embodiment 1, wherein the plurality of single-strand-nucleic-acid imaging molecules are fluorogenic.

Embodiment 14: The method of Embodiment 1, wherein:
the plurality of single-strand-nucleic-acid imaging molecules are fluorescent; and
the detected change in light is fluorescence emitted by the single-strand-nucleic-acid imaging molecules.

Embodiment 15: The method of Embodiment 1, wherein the single-strand-nucleic-acid imaging molecules are detected individually in order to generate a single-molecule localization super-resolution microscopy image.

Embodiment 16: The method of Embodiment 1, wherein the sample is a biological tissue section.

Embodiment 17: The method of Embodiment 1, wherein:
the plurality of targets are antibodies or binding ligands that bind to a plurality of specific proteins in the sample; and
each type of antibody or binding ligand is conjugated to a different single-strand nucleic acid.

Embodiment 18: The method of Embodiment 1, wherein the single-strand nucleic acids are RNA or DNA molecules.

Embodiment 19: The method of Embodiment 1, wherein the single-strand-nucleic-acid imaging molecules comprise a single-strand nucleic acid coupled to a molecule exhibiting a Raman signature detectable by a Raman microscopy.

Embodiment 20: The method of Embodiment 1, wherein the single-strand-nucleic-acid imaging molecules comprise a single-strand nucleic acid coupled to a nanoparticle.

Embodiment 21: The method of Embodiment 20, wherein the nanoparticle is a gold nanoparticle.

Embodiment 22: The method of Embodiment 20, wherein the interaction is scattering.

Embodiment 23: A kit comprising:
a plurality of transient single-strand-nucleic-acid adapter molecules; and
a plurality of single-strand-nucleic-acid imaging molecules; and
wherein the transient single-strand-nucleic-acid adapter molecules comprise:
   a first region having a target-complementary sequence; and
   a second region having a single-strand-nucleic-acid-imaging-molecule-complementary sequence.

Embodiment 24: The kit of Embodiment 23, wherein the plurality of transient single-strand-nucleic-acid adapter molecules have a quantity or concentration greater than the plurality of single-strand-nucleic-acid imaging molecules.

Embodiment 25: The kit of Embodiment 23, wherein the plurality of transient single-strand-nucleic-acid adapter molecules have a quantity or concentration greater than the plurality of single-strand-nucleic-acid imaging molecules by a ratio selected from the group consisting of: at least about 1; at least about 10; and at least about 100.

Embodiment 26: The kit of Embodiment 23, wherein the plurality of transient non-fluorescent single-strand-nucleic-acid adapter molecules have a quantity or concentration greater than the plurality of fluorescent imaging molecules by a ratio of about 500.

Embodiment 27: An imaging method, the method comprising:
performing a first labeling, which comprises:
   applying to a sample one or more targets comprising a first target, which comprises a first target single-strand-nucleic-acid;
   applying to the sample a first adapter comprising a first adapter single-strand-nucleic-acid; and
   applying to the sample a first imaging molecule comprising a first imaging molecule single-strand-nucleic-acid and a first detection motif; and
acquiring a first image of the first detection motif,
wherein the first adapter single-strand-nucleic-acid comprises:
   a first region having a sufficient sequence complementarity to bind the target single-strand-nucleic-acid; and
   a second region having a sufficient sequence complementarity to bind the first imaging molecule single-strand-nucleic-acid, and
wherein the first adapter binds the target and the first imaging molecule.

Embodiment 28: The method of Embodiment 27, wherein at least one of the following applies:
(a) the first target comprises the first target single-strand-nucleic-acid attached to an antibody or a polypeptide that specifically binds to a point of interest, optionally a protein, a protein complex, a nucleic acid, a cell structure, a cell organelle, or a cell, in the sample;
(b) the first target comprises the first target single-strand-nucleic-acid attached to a targeting nucleic acid that specifically binds to or is complementary with a point of interest, optionally a nucleic acid, in the sample.

Embodiment 29: The method of Embodiment 27, wherein at least one of the following applies:
(a) the first detection motif is a fluorescence motif, optionally a fluorescent protein, a fluorescent small molecule, or a quantum dot,
(b) the first detection motif is a metal nanoparticle, optionally a gold nanoparticle,
(c) the first detection motif is a Raman scattering motif, optionally a Raman dye, optionally a Raman dye suitable for a stimulated Raman scattering microscopy,
(d) the first detection motif is an isotope.

Embodiment 30: The method of Embodiment 27, wherein at least one of the following applies:
(a) a number of complementary base pairs between the first target single-strand-nucleic-acid and the first region of the first adapter ranges between 1-30,
(b) a number of complementary base pairs between the first target single-strand-nucleic-acid and the first region of the first adapter ranges between 5-20,
(c) a number of complementary base pairs between the first target single-strand-nucleic-acid and the first region of the first adapter ranges between 8-12.

Embodiment 31: The method of Embodiment 27, wherein at least one of the following applies:
(a) a Kₒₙ between the first target single-strand-nucleic-acid and the first region of the adapter ranges between 1*10⁴ l/M*s and 1*10⁷ l/M*s,
(b) a K_{off} between the first target single-strand-nucleic-acid and the first region of the adapter ranges between 1 l/s and 0.0001 l/s,
(c) a K_{d} between the first target single-strand-nucleic-acid and the first region of the adapter ranges between 10 μM and 1 nM.

Embodiment 32: The method of Embodiment 27, wherein at least one of the following applies:
(a) a number of complementary base pairs between the first imaging molecule single-strand-nucleic-acid and the second region of the adapter ranges between 1 and 30,
(b) a number of complementary base pairs between the first imaging molecule single-strand-nucleic-acid and the second region of the adapter ranges between 5 and 20,
(c) a number of complementary base pairs between the first imaging molecule single-strand-nucleic-acid and the second region of the adapter ranges between 8 and 12.

Embodiment 33: The method of Embodiment 27, wherein at least one of the following applies:
(a) a Kₒₙ between the first imaging molecule single-strand-nucleic-acid and the second region of the adapter ranges between 1*10⁴ l/M*s and 1*10⁷ l/M*s,
(b) a K_{off} between the first imaging molecule single-strand-nucleic-acid and the second region of the adapter ranges between 1000 l/s and 0.0001 l/s,
(a) a K_{d} between the first imaging molecule single-strand-nucleic-acid and the second region of the adapter ranges between 10 UM and 1 nM.

Embodiment 34: The method of Embodiment 27, the method comprising:
performing the first labeling, which comprises:
   applying to the sample a plurality of first targets, each comprising a first target single-strand-nucleic-acid;
   applying to the sample a plurality of first adapters, each comprising a first adapter single-strand-nucleic-acid; and
   applying to the sample a plurality of first imaging molecules, each comprising a first imaging molecule single-strand-nucleic-acid and a first detection motif; and
acquiring the first image of the plurality of first detection motifs of the plurality of first imaging molecules,
wherein each of the first adapters mediates an association of each of the plurality of first targets and each of the plurality of the first imaging molecules in a sequence-specific manner, and
wherein the plurality of first detection motifs do not interfere with each other during the acquisition of the first image.

Embodiment 35: The method of Embodiment 27, wherein at least one of the following applies:
(a) the method further comprises:
applying to the sample an eraser molecule to disrupt the association between the first target and the first imaging molecule mediated by the first adapter;
performing a second labeling, which comprises:
   applying to a sample a second target comprising a second target single-strand-nucleic-acid;
   applying to the sample a second adapter comprising a second adapter single-strand-nucleic-acid; and
   applying to the sample a second imaging molecule comprising a second imaging molecule single-strand-nucleic-acid and a second detection motif; and
acquiring a second image of the second detection motif,
(b) the one or more targets applied in the first labeling further comprises a second target comprising a second single-strand-nucleic-acid, and the method further comprises:
applying to the sample an eraser molecule to disrupt the association between the first target and the first imaging molecule mediated by the first adapter;
performing a second labeling, which comprises:
   applying to the sample a second adapter comprising a second adapter single-strand-nucleic-acid; and
   applying to the sample a second imaging molecule comprising a second imaging molecule single-strand-nucleic-acid and a second detection motif; and
acquiring a second image of the second detection motif,
wherein, for (a) and (b), the second adapter single-strand-nucleic-acid comprises:
   a third region having a sufficient complementarity to bind the second target single-strand-nucleic-acid; and
   a fourth region having a sufficient complementarity to bind the second imaging molecule single-strand-nucleic-acid, and
wherein, for (a) and (b), the second adapter mediates an association between the second target and the second imaging molecule.

Embodiment 36: The method of Embodiment 35, wherein
the eraser molecule comprises an eraser molecule single-strand-nucleic-acid having a sufficient sequence complementarity to bind the first region or the second region of the first adapter, and
the eraser molecule
   prevents the hybridization between the first target single-strand-nucleic-acid and the first region of the adapter, or
   prevents the hybridization between the first imaging molecule single-strand-nucleic-acid and the second region of the adapter.

Embodiment 37: The method of Embodiment 35, wherein at least one of the following applies:
(a) the first target, the first adapter, the first imaging molecule and the eraser molecule are not washed away from the sample before the application of the second target, the second adapter, and the second imaging molecule,
(b) the one or more targets, the first adapter, the first imaging molecule and the eraser molecule are not washed away from the sample before the application of the second adapter, and the second imaging molecule.

Embodiment 38: The method of Embodiment 35, wherein a signal of the first detection motif and a signal of the second detection motif overlap or are the same.

Embodiment 39: The method of Embodiment 35, wherein in each of the first labeling and the second labeling, 4 or more of different detection motifs having different signals are used. Embodiment 40: The method of Embodiment 35, wherein the sample is expanded according to an expansion microscopy technology.

Embodiment 41: A device, comprising:
a sample holder for holding a sample;
a computer-operated liquid applicator for applying a liquid to the sample;
a computer-operated microscope; and
a computer,
wherein the computer is programmed to perform the following operations:
(a) operate the liquid applicator to perform a first application of:
   one ore more targets, which comprises a first target for specifically binding to a first component in the sample;
   a first imaging molecule comprising a first detection motif detectable by the microscope; and
   a first adapter for mediating an association between the first target and the first imaging molecule,
(b) operate the microscope to record a first signal of the first detection motif,
(c) operate the liquid applicator to perform a second application of (c1) or (c2):
   (c1) the second application comprises the application of:
      an eraser molecule for interrupting the first adapter-mediated interaction between the first target and the first adapter;
      a second target for specifically binding to a second component in the sample;
      a second imaging molecule comprising a second detection motif detectable by the microscope; and
      a second adapter for mediating an association between the second target and the second imaging molecule,
   (c2) the one or more targets applied in (a) further comprises a second target for specifically binding to a second component in the sample, and the second application comprises the application of:
      an eraser molecule for interrupting the first adapter-mediated interaction between the first target and the first adapter;
      a second imaging molecule comprising a second detection motif detectable by the microscope; and
      a second adapter for mediating an association between the second target and the second imaging molecule,
(d) operate the microscope to record a second signal of the second detection motif,
wherein the computer is programmed to perform operations (a), (b), (c) and (d) sequentially in this order.

Embodiment 42: The device of Embodiment 41, wherein the device does not remove the liquid applied in operation (a) before performing operations (c) and (d).

Embodiment 43: The device of Embodiment 41, wherein the first signal and the second signal overlap with each other or are identical.

Embodiment 44: The device of Embodiment 41, wherein the first detection motif and the second detection motif are
(a) the first detection motif or the second detection motif is a fluorescence motif, optionally a fluorescent protein, a fluorescent small molecule, or a quantum dot,
(b) the first detection motif or the second detection motif is a metal nanoparticle, optionally a gold nanoparticle,
(c) the first detection motif or the second detection motif is a Raman scattering motif, optionally a Raman dye, optionally a Raman dye suitable for a stimulated Raman scattering microscopy,
(d) the first detection motif or the second detection motif is an isotope.

Embodiment 45: The device of Embodiment 41, further comprising a reservoir for storing the one or more targets, the first adapter, the first imaging molecule, the eraser, the second target, the second adapter, the second imaging molecule.

Embodiment 46: The device of Embodiment 41, further comprising at least one selected from the group consisting of the first target, the first adapter, the first imaging molecule, the eraser, the second target, the second adapter, and the second imaging molecule, wherein
the first target comprises a first target single-strand-nucleic-acid;
the first adapter comprises a first adapter single-strand-nucleic-acid;
the first imaging molecule comprises a first imaging molecule single-strand-nucleic-acid attached to the first detection motif;
the eraser molecule comprises an eraser molecule single-strand-nucleic-acid having a sufficient sequence complementarity to bind the first region or the second region of the first adapter;
the second target comprises a second target single-strand-nucleic-acid;
the second adapter comprises a second adapter single-strand-nucleic-acid;
the second imaging molecule comprises a second imaging molecule single-strand-nucleic-acid attached to the second detection motif;
wherein the first adapter single-strand-nucleic-acid comprises:
   a first region having a sufficient sequence complementarity to bind the first target single strand nucleic acid; and
   a second region having a sufficient sequence complementarity to bind the first imaging molecule single-strand-nucleic-acid, and
wherein the second adapter single-strand-nucleic-acid comprises:
   a third region having a sufficient sequence complementarity to bind the second target single strand nucleic acid; and
   a fourth region having a sufficient sequence complementarity to bind the second imaging molecule single-strand-nucleic-acid.

Embodiment 47: The device of Embodiment 46, which comprises the first target, the first adapter, the first imaging molecule, the eraser, the second target, the second adapter, and the second imaging molecule.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of microscopy imaging, the method comprising:
exposing a sample having a plurality of targets to a plurality of transient single-strand-nucleic-acid adapter molecules; and
exposing the sample to a plurality of single-strand-nucleic-acid imaging molecules; and
exposing the sample to an illumination source having a wavelength capable of interacting with the plurality of single-strand-nucleic-acid imaging molecules;
wherein the transient single-strand-nucleic-acid adapter molecules comprise:
a first region having a target-complementary sequence; and
a second region having a single-strand-nucleic-acid-imaging-molecule-complementary sequence.

2. The method of claim 1, wherein the plurality of transient single-strand-nucleic-acid adapter molecules have a quantity greater than an estimated or actual quantity of targets.

3. The method of claim 1, wherein the plurality of transient single-strand-nucleic-acid adapter molecules have a quantity or concentration greater than the plurality of single-strand-nucleic-acid imaging molecules.

4. The method of claim 1, wherein the plurality of transient single-strand-nucleic-acid adapter molecules have a quantity or concentration greater than the plurality of single-strand-nucleic-acid imaging molecules by a ratio selected from the group consisting of: at least about 1; at least about 10; and at least about 100.

5. The method of claim 1, wherein the plurality of transient single-strand-nucleic-acid adapter molecules have a quantity or concentration greater than the plurality of single-strand-nucleic-acid imaging molecules by a ratio of about 500.

6. The method of claim 1, wherein the target-complementary sequence is less than 11 nucleotides.

7. The method of claim 1, wherein the target-complementary sequence is selected from the group consisting of: between 6 and 10 nucleotides and between 8 and 10 nucleotides.

8. The method of claim 1, further comprising:
exposing the sample to an eraser molecule adapted and configured to quench the transient single-strand-nucleic-acid adapter molecules;
exposing the sample to a second plurality of transient single-strand-nucleic-acid adapter molecules having a second, different target-complementary sequence; and
exposing the sample to the plurality of single-strand-nucleic-acid imaging molecules; and
exposing the sample to an illumination source having a wavelength capable of interacting with the plurality of single-strand-nucleic-acid imaging molecules.

9. The method of claim 8, wherein the method is performed without rinsing the plurality of transient single-strand-nucleic-acid adapter molecules from the sample.

10. The method of claim 8, wherein the eraser molecule and the second plurality of transient single-strand-nucleic-acid adapter molecules are introduced simultaneously.

11. The method of claim 8, wherein the eraser molecule and the second plurality of transient single-strand-nucleic-acid adapter molecules are introduced sequentially.

12. The method of claim 1, wherein the plurality of single-strand-nucleic-acid imaging molecules include a speed-optimized sequence.

13. The method of claim 1, wherein the plurality of single-strand-nucleic-acid imaging molecules are fluorogenic.

14. The method of claim 1, wherein:
the plurality of single-strand-nucleic-acid imaging molecules are fluorescent; and
the detected change in light is fluorescence emitted by the single-strand-nucleic-acid imaging molecules.

15. The method of claim 1, wherein the single-strand-nucleic-acid imaging molecules are detected individually in order to generate a single-molecule localization super-resolution microscopy image.

16. The method of claim 1, wherein the sample is a biological tissue section.

17. The method of claim 1, wherein:
the plurality of targets are antibodies or binding ligands that bind to a plurality of specific proteins in the sample; and
each type of antibody or binding ligand is conjugated to a different single-strand nucleic acid.

18. The method of claim 1, wherein the single-strand nucleic acids are RNA or DNA molecules.

19. The method of claim 1, wherein the single-strand-nucleic-acid imaging molecules comprise a single-strand nucleic acid coupled to a molecule exhibiting a Raman signature detectable by a Raman microscopy.

20. The method of claim 1, wherein the single-strand-nucleic-acid imaging molecules comprise a single-strand nucleic acid coupled to a nanoparticle.

21. The method of claim 20, wherein the nanoparticle is a gold nanoparticle.

22. The method of claim 20, wherein the interaction is scattering.

23. A kit comprising:
a plurality of transient single-strand-nucleic-acid adapter molecules; and
a plurality of single-strand-nucleic-acid imaging molecules; and
wherein the transient single-strand-nucleic-acid adapter molecules comprise:
a first region having a target-complementary sequence; and
a second region having a single-strand-nucleic-acid-imaging-molecule-complementary sequence.

24. The kit of claim 23, wherein the plurality of transient single-strand-nucleic-acid adapter molecules have a quantity or concentration greater than the plurality of single-strand-nucleic-acid imaging molecules.

25. The kit of claim 23, wherein the plurality of transient single-strand-nucleic-acid adapter molecules have a quantity or concentration greater than the plurality of single-strand-nucleic-acid imaging molecules by a ratio selected from the group consisting of: at least about 1; at least about 10; and at least about 100.

26. The kit of claim 23, wherein the plurality of transient non-fluorescent single-strand-nucleic-acid adapter molecules have a quantity or concentration greater than the plurality of fluorescent imaging molecules by a ratio of about 500.

27. An imaging method, the method comprising:
performing a first labeling, which comprises:
applying to a sample one or more targets comprising a first target, which comprises a first target single-strand-nucleic-acid;
applying to the sample a first adapter comprising a first adapter single-strand-nucleic-acid; and
applying to the sample a first imaging molecule comprising a first imaging molecule single-strand-nucleic-acid and a first detection motif; and
acquiring a first image of the first detection motif,
wherein the first adapter single-strand-nucleic-acid comprises:
a first region having a sufficient sequence complementarity to bind the target single-strand-nucleic-acid; and
a second region having a sufficient sequence complementarity to bind the first imaging molecule single-strand-nucleic-acid, and
wherein the first adapter binds the target and the first imaging molecule.

28. The method of claim 27, wherein at least one of the following applies:
(a) the first target comprises the first target single-strand-nucleic-acid attached to an antibody or a polypeptide that specifically binds to a point of interest, optionally a protein, a protein complex, a nucleic acid, a cell structure, a cell organelle, or a cell, in the sample;
(b) the first target comprises the first target single-strand-nucleic-acid attached to a targeting nucleic acid that specifically binds to or is complementary with a point of interest, optionally a nucleic acid, in the sample.

29. The method of claim 27, wherein at least one of the following applies:
(a) the first detection motif is a fluorescence motif, optionally a fluorescent protein, a fluorescent small molecule, or a quantum dot,
(b) the first detection motif is a metal nanoparticle, optionally a gold nanoparticle,
(c) the first detection motif is a Raman scattering motif, optionally a Raman dye, optionally a Raman dye suitable for a stimulated Raman scattering microscopy,
(d) the first detection motif is an isotope.

30. The method of claim 27, wherein at least one of the following applies:
(a) a number of complementary base pairs between the first target single-strand-nucleic-acid and the first region of the first adapter ranges between 1-30,
(b) a number of complementary base pairs between the first target single-strand-nucleic-acid and the first region of the first adapter ranges between 5-20,
(c) a number of complementary base pairs between the first target single-strand-nucleic-acid and the first region of the first adapter ranges between 8-12.

31. The method of claim 27, wherein at least one of the following applies:
(a) a Kₒₙ between the first target single-strand-nucleic-acid and the first region of the adapter ranges between 1*10⁴ l/M*s and 1*10₇ l/M*s,
(b) a K_{off} between the first target single-strand-nucleic-acid and the first region of the adapter ranges between 1 l/s and 0.0001 l/s,
(c) a K_{d} between the first target single-strand-nucleic-acid and the first region of the adapter ranges between 10 μM and 1 nM.

32. The method of claim 27, wherein at least one of the following applies:
(a) a number of complementary base pairs between the first imaging molecule single-strand-nucleic-acid and the second region of the adapter ranges between 1 and 30,
(b) a number of complementary base pairs between the first imaging molecule single-strand-nucleic-acid and the second region of the adapter ranges between 5 and 20,
(c) a number of complementary base pairs between the first imaging molecule single-strand-nucleic-acid and the second region of the adapter ranges between 8 and 12.

33. The method of claim 27, wherein at least one of the following applies:
(a) a Kₒₙ between the first imaging molecule single-strand-nucleic-acid and the second region of the adapter ranges between 1*10⁴ l/M*s and 1*10⁷ l/M*s,
(b) a K_{off} between the first imaging molecule single-strand-nucleic-acid and the second region of the adapter ranges between 1000 l/s and 0.0001 l/s,
(a) a K_{d} between the first imaging molecule single-strand-nucleic-acid and the second region of the adapter ranges between 10 UM and 1 nM.

34. The method of claim 27, the method comprising:
performing the first labeling, which comprises:
applying to the sample a plurality of first targets, each comprising a first target single-strand-nucleic-acid;
applying to the sample a plurality of first adapters, each comprising a first adapter single-strand-nucleic-acid; and
applying to the sample a plurality of first imaging molecules, each comprising a first imaging molecule single-strand-nucleic-acid and a first detection motif; and
acquiring the first image of the plurality of first detection motifs of the plurality of first imaging molecules,
wherein each of the first adapters mediates an association of each of the plurality of first targets and each of the plurality of the first imaging molecules in a sequence-specific manner, and
wherein the plurality of first detection motifs do not interfere with each other during the acquisition of the first image.

35. The method of claim 27, wherein at least one of the following applies:
(a) the method further comprises:
applying to the sample an eraser molecule to disrupt the association between the first target and the first imaging molecule mediated by the first adapter;
performing a second labeling, which comprises:
applying to a sample a second target comprising a second target single-strand-nucleic-acid;
applying to the sample a second adapter comprising a second adapter single-strand-nucleic-acid; and
applying to the sample a second imaging molecule comprising a second imaging molecule single-strand-nucleic-acid and a second detection motif, and
acquiring a second image of the second detection motif,
(b) the one or more targets applied in the first labeling further comprises a second target comprising a second single-strand-nucleic-acid, and the method further comprises:
applying to the sample an eraser molecule to disrupt the association between the first target and the first imaging molecule mediated by the first adapter;
performing a second labeling, which comprises:
applying to the sample a second adapter comprising a second adapter single-strand-nucleic-acid; and
applying to the sample a second imaging molecule comprising a second imaging molecule single-strand-nucleic-acid and a second detection motif; and
acquiring a second image of the second detection motif,
wherein, for (a) and (b), the second adapter single-strand-nucleic-acid comprises:
a third region having a sufficient complementarity to bind the second target single-strand-nucleic-acid; and
a fourth region having a sufficient complementarity to bind the second imaging molecule single-strand-nucleic-acid, and
wherein, for (a) and (b), the second adapter mediates an association between the second target and the second imaging molecule.

36. The method of claim 35, wherein
the eraser molecule comprises an eraser molecule single-strand-nucleic-acid having a sufficient sequence complementarity to bind the first region or the second region of the first adapter, and
the eraser molecule
prevents the hybridization between the first target single-strand-nucleic-acid and the first region of the adapter, or
prevents the hybridization between the first imaging molecule single-strand-nucleic-acid and the second region of the adapter.

37. The method of claim 35, wherein at least one of the following applies:
(a) the first target, the first adapter, the first imaging molecule and the eraser molecule are not washed away from the sample before the application of the second target, the second adapter, and the second imaging molecule,
(b) the one or more targets, the first adapter, the first imaging molecule and the eraser molecule are not washed away from the sample before the application of the second adapter, and the second imaging molecule.

38. The method of claim 35, wherein a signal of the first detection motif and a signal of the second detection motif overlap or are the same.

39. The method of claim 35, wherein in each of the first labeling and the second labeling, 4 or more of different detection motifs having different signals are used.

40. The method of claim 35, wherein the sample is expanded according to an expansion microscopy technology.

41. A device, comprising:
a sample holder for holding a sample;
a computer-operated liquid applicator for applying a liquid to the sample;
a computer-operated microscope; and
a computer,
wherein the computer is programmed to perform the following operations:
(a) operate the liquid applicator to perform a first application of:
one ore more targets, which comprises a first target for specifically binding to a first component in the sample;
a first imaging molecule comprising a first detection motif detectable by the microscope; and
a first adapter for mediating an association between the first target and the first imaging molecule,
(b) operate the microscope to record a first signal of the first detection motif,
(c) operate the liquid applicator to perform a second application of (c1) or (c2):
(c1) the second application comprises the application of:
an eraser molecule for interrupting the first adapter-mediated interaction between the first target and the first adapter,
a second target for specifically binding to a second component in the sample;
a second imaging molecule comprising a second detection motif detectable by the microscope; and
a second adapter for mediating an association between the second target and the second imaging molecule,
(c2) the one or more targets applied in (a) further comprises a second target for specifically binding to a second component in the sample, and the second application comprises the application of:
an eraser molecule for interrupting the first adapter-mediated interaction between the first target and the first adapter;
a second imaging molecule comprising a second detection motif detectable by the microscope; and
a second adapter for mediating an association between the second target and the second imaging molecule,
(d) operate the microscope to record a second signal of the second detection motif,
wherein the computer is programmed to perform operations (a), (b), (c) and (d) sequentially in this order.

42. The device of claim 41, wherein the device does not remove the liquid applied in operation (a) before performing operations (c) and (d).

43. The device of claim 41, wherein the first signal and the second signal overlap with each other or are identical.

44. The device of claim 41, wherein the first detection motif and the second detection motif are
(a) the first detection motif or the second detection motif is a fluorescence motif, optionally a fluorescent protein, a fluorescent small molecule, or a quantum dot,
(b) the first detection motif or the second detection motif is a metal nanoparticle, optionally a gold nanoparticle,
(c) the first detection motif or the second detection motif is a Raman scattering motif, optionally a Raman dye, optionally a Raman dye suitable for a stimulated Raman scattering microscopy,
(d) the first detection motif or the second detection motif is an isotope.

45. The device of claim 41, further comprising a reservoir for storing the one or more targets, the first adapter, the first imaging molecule, the eraser, the second target, the second adapter, the second imaging molecule.

46. The device of claim 41, further comprising at least one selected from the group consisting of the first target, the first adapter, the first imaging molecule, the eraser, the second target, the second adapter, and the second imaging molecule, wherein
the first target comprises a first target single-strand-nucleic-acid;
the first adapter comprises a first adapter single-strand-nucleic-acid;
the first imaging molecule comprises a first imaging molecule single-strand-nucleic-acid attached to the first detection motif;
the eraser molecule comprises an eraser molecule single-strand-nucleic-acid having a sufficient sequence complementarity to bind the first region or the second region of the first adapter;
the second target comprises a second target single-strand-nucleic-acid;
the second adapter comprises a second adapter single-strand-nucleic-acid;
the second imaging molecule comprises a second imaging molecule single-strand-nucleic-acid attached to the second detection motif;
wherein the first adapter single-strand-nucleic-acid comprises:
a first region having a sufficient sequence complementarity to bind the first target single strand nucleic acid; and
a second region having a sufficient sequence complementarity to bind the first imaging molecule single-strand-nucleic-acid, and
wherein the second adapter single-strand-nucleic-acid comprises:
a third region having a sufficient sequence complementarity to bind the second target single strand nucleic acid; and
a fourth region having a sufficient sequence complementarity to bind the second imaging molecule single-strand-nucleic-acid.

47. The device of claim 46, which comprises the first target, the first adapter, the first imaging molecule, the eraser, the second target, the second adapter, and the second imaging molecule.
